SEQUENCE LISTING (12 sequences)

SEQ ID NO: 1 - DNA, 1040 bp, Homo sapiens
taggtaggggctctatctgcatctttctttctttttttctttctttcccttcctcccttcctcactccctcggtcctctctttctttccttttctttcttccttcctcccttcctccctccctccctctctctttctctctttctttctttccttctttctttctttctctcttccttccctccctccctccttccttcctttctctttctttctctttctttctttttttccttccttccttccttctttctctttctctccctcccttccttccttccttccttccttccttcctttctttctttctttctttctttctttctttctttctttctttctttctttcttccttccttccttccttccttccttccttccttccttcctttcttttctttctttctctttctttttgagacagagctcttattacccatgctggagtgcagtggtgtgaccttggcttactgcaacatctgcctcctagggtcaagtgattctcctgcctcagcctcctaagtagctgggattacagacacatgccaccacacccaatatttatttttattaaaattttttttaaaattatttttaaaaaattaaaaataattttgtatttttagtagagacggggtttctccatgttggtcaggctgatctcaaactcccaacctcaggtgatcctcccacctcacctcccaaagtgctgggattacaggcatgagccaccgtgcccagcctggttcctggtttctaagacatcacacacacacacacacacacacacacacactcacacactcagagagagagagagagagaggatcattaagacatgatacactaagaaattctattctgcagacactgagaatccgttaaaaagtttgaagggaagaattgagatcatcaggtgtttatttgaggaaattgtctgtggttgaactatcctttcctttctctccctgagatttggtcttctcaattagaagcgttgcacaattcccccaacctccatacatacggcagctcttctag

SEQ ID NO: 2 - DNA, 646 bp, Homo sapiens
taggtaggggctctatctgcatctttctttctttttttctttctttcccttcctcccttcctcactccctcggtcctctctttctttccttttctttcttccttcctcccttcctccctccctccctctctctttctctctttctttctttccttctttctttctttctctcttccttccctccctccctccttccttcctttctctttctttctctttctttctttttttccttccttccttccttctttctctttctctccctcccttccttccttccttccttccttccttcctttctttctttctttctttctttctttctttctttctttctttctttctttcttccttccttccttccttccttccttccttccttccttcctttcttttctttctttctctttctttttgagacagagctcttattacccatgctggagtgcagtggtgtgaccttggcttactgcaacatctgcctcctagggtcaagtgattctcctgcctcagcctcctaagtagctgggattacagacacatgccaccacacccaatatttatttttattaaaattttttttaaaattatttttaaaaaattaaaaataattttgtatttttagtagagacggggtttctccatg

SEQ ID NO: 3 - DNA, 5033 bp, Homo sapiens
ttccatccttttctcctcacaggaactcacgagcctggggagaattcgtcatccccacatggaacctcagtatggccaacaggcagccctctggtagaaaaatcaacaatcagtcctgcaggtatgctccaatctcatctcctccacattgttatggtctcccggagctttgttaatctctttgaagacgaacatttttgatcttttccataggggaagtgcaagggacgcctgtgttggagtttgctccccagctacagggctccctaactttttatttaacctccagctttatgaccatgtacaagtatgcaattctaatctttatcttgttttgagattctaatccagagtagttgggggacaggaagcccagcctataacccctgatttttttttttttttttgagacagagtctcattctgtcacccaggctggagtgcagtggcacaatctcagctgactgcaacccctgcctcccgagtttaagcaattttggtgtctcagcctcctgagtagctgggattacaggcgcctgccaccacccccggctaatttttgtattttttggtagagacggagtttcaccatgttggtcaggctggtcttgaactcctgacctcaagtaatctgctctcctcaggactctcttggaaggatttttttttttttttttttttttagatggaattctgctctttgtctcccaggctggagtgcaatggtgcgatcttggctcactgtaacctccgcctcccaggttcaagtgattctcctgtctcagcctccggagtagctggaattacagccacccgccaccacgcccagctagtttttgtatttttagtagagacggggtttaaccatgttggccaggctggtcttgaactcctgacctcaggtgatccgcccgctttggcctcccaaagtgctgggattacaggcgtgagccactgcgcccagcaggaactcttagttcagaagaagagtgagctaataatgtatatgtcttatagcacttttttgcatctaagttgttttatttgggatcagaagacttttattttcttttttaaaatttagcttttattttaagtttaggggtacatgtgcaggcttgttatataggtaaacttttgccattggggttggttttacagattatttcatcacccaggtattaagcctagtatccattagttatttttcctgatcttctccctcctcccaccctccaccctccaataggcccttaattaaactaaagagcttccgcacagtaaaggaaactatcaacagagtaaacagacaatctacagaatgggagaaaagctttacaaactttgcatctgacaaaggtctaatatccagcatctaaaagaaacgtgccgggtgtggtggctcacacctgtaatcccagcactttggcggatcacctgaggttgggagttcgagaccagcctgcccaacatggagaaagccaatctctactaaaaatacaaaaattagccgggcatggtggcacatgcctgtaattccagctactccaggggctgaggcagaagaatcgcttgaacccggaaggtggaggttgcagtgagccgagattgcaccactgcactccagcctgggtgacagagtgagactccatctcaaaaaaaataaaaaataaaaaataaacgaatttataatttaaaaaaccagcaaaaaacaaaacaaacaaacaaaagaaacattacacagtgggcaaaggacgtggttcttcaatcattttgacctggataaaattggtagctcctgagtagaaaagaagattgggaaagcatgaggaatgaattatagtttcacttaccccaccggcctagggaaaaactcaattcctaggagacgcagcttagaaccaaggtgggcctcatcttgagagaaatccatgagccatgcctatcttagtctgtattgcattgcttatagcagaacagctgaaactgggtaatttacaaagaaaaataatgtatctattacagatatggaggctggaaagttgaaatccagaggggcacacctggtgagagccctgttgctggtggggactctgtggcatccagaggtagacagggcatcacacgtcaagacagctgagtgcgctaatgtgatagcacaggtctcacttcctattttaaagcttttcttttctttttttgtttgagatggagtctcaatctgtcacccaggatggagtgcagtgctgcgatcttggctcactgcaacctccacctcctgggttcaagtgattttcctctcttagcctcctgagtagctgggaccacaggtgcgcaccactatgtctggctaatttttgtatttttttgtagagacatggtttcaccatgttggccaggctggtctcgaattcctgacttcaagtaatccacccacctcagcctcccaaagtgctgggattacaagcatgagccaccatgcatggcctaaagcttcttttaaagccaccaagtcccttcccatgttagcccactaatccatgggttagtcatgaatggattaatctattcatacggacagagccctcatcacccaatcacctcttaaaggccccacctctcaatactgccacactggggattaagtttcaacagagttttggaggggacattcaaatcatagtaatgcccaaagtgaaaaatcttccctgcacttttccctcaacaaaaacagccagagatagtgagctgccaggaaattcttttttttttcctcttctgtcctaaatcagcatcgctagacctttacatgattcaacctcatcttcttcaccctctgggtcatgaaattttatttatttatttattattttcttgggacagactctggctctgtcgcccaggctgaagtgcagtggtgtgatcttggctcactgcaacctccgcctcccgggttcaagcgattctcctgcctcagcctcctgagtagctgggattacaggtgggcgccaccacacccagctaattttttgtatttttagtagagatggggtttcaccatattagccaggatggtctccatctcttgacctcgtgatctgcccacctcagcctcccaaaatgctgggattacaggcatgagacaccacgcccagcaggccagggtcatgagattttaatcaagagcaacttccactgattcctgagagtgcatctgtgggcccctgctctgatctgaacagaagtgccgtgtcttctctgacctccacttctcaattcaagagccttagtatctgccagtatcacacactgagcattagctccatctcatgggggtgtaggtaggggctctatctgcatctttctttctttttttctttctttcccttcctcccttcctcactccctcggtcctctctttctttccttttctttcttccttcctcccttcctccctccctccctctctctttctctctttctttctttccttctttctttctttctctcttccttccctccctccctccttccttcctttctctttctttctctttctttctttttttccttccttccttccttctttctctttctctccctcccttccttccttccttccttccttccttcctttctttctttctttctttctttctttctttctttctttctttctttctttcttccttccttccttccttccttccttccttccttccttcctttcttttctttctttctctttctttttgagacagagctcttattacccatgctggagtgcagtggtgtgaccttggcttactgcaacatctgcctcctagggtcaagtgattctcctgcctcagcctcctaagtagctgggattacagacacatgccaccacacccaatatttatttttattaaaattttttttaaaattatttttaaaaaattaaaaataattttgtatttttagtagagacggggtttctccatgttggtcaggctgatctcaaactcccaacctcaggtgatcctcccacctcacctcccaaagtgctgggattacaggcatgagccaccgtgcccagcctggttcctggtttctaagacatcacacacacacacacacacacacacacacactcacacactcagagagagagagagagagaggatcattaagacatgatacactaagaaattctattctgcagacactgagaatccgttaaaaagtttgaagggaagaattgagatcatcaggtgtttatttgaggaaattgtctgtggttgaactatcctttcctttctctccctgagatttggtcttctcaattagaagcgttgcacaattcccccaacctccatacatacggcagctcttctagacacaggttttcccaggtcaaatgcggggaccccagccatatctcccaccctgagaaattttggagtttcagggagctcagaagctctgcagaggccaccctctctgaggggattcttcttagacctccatccagaggcaaatgttgacctgtccatgctgaaaccctcaggccttcctgggtcatcttctcccacccgctccttgatgacagggagcaggagcactaaagccacaccagaaatggattcaggactgacaggagccaccttgtcacctaagacatctacaggtgcaatcgtggtgacagaacatactctgccctttacttccccagataagaccttggccagtcctacatcttcggttgtgggaagaaccacccagtctttgggggtgatgtcctctgctctccctgagtcaacctctagaggaatgacacactccgagcaaagaaccagcccatcgctgagtccccaggtcaatggaactccctctaggaactaccctgctacaagcatggtttcaggattgagttccccaaggaccaggaccagttccacagaaggaaattttaccaaagaagcatctacatacacactcactgtagagaccacaagtggcccagtcactgagaagtacacagtccccactga

SEQ ID NO: 4 - DNA, 22 bp, Artificial Sequence
  Synthetic Construct
acaaactagcaaaataggctgt

SEQ ID NO: 5 - DNA, 22 bp, Artificial Sequence
  Synthetic Construct
cttcttaatgtttttggcatct

SEQ ID NO: 6 - DNA, 20 bp, Artificial Sequence
  Synthetic Construct
aagaaggtggtgaagcaggc

SEQ ID NO: 7 - DNA, 20 bp, Artificial Sequence
  Synthetic Construct
tccaccaccctgttgctgta

SEQ ID NO: 8 - DNA, 870 bp, Human adenovirus type 5
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 9 - DNA, 471 bp, Human adenovirus type 5
atggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 10 - DNA, 1557 bp, Human adenovirus type 5
atggccagccgcgaaggaaatcagttaagcgaccgccacagggagcacacccccgaacgcggaaggggttctgccagtcatccgccttccaggtcggaccgcagcccttcgcaatcgcctccacctctgcccccgaagagaaacacctgtcgccgtgtggggtcaggttcttctaccgacagtcagttggtaatggtgagcgaaacttcacaatcctccctgtctccggaacggagcgacagtcctcctcccccgataccccccaaaaagaagccaaggaagaccaaacatgttcccatgcaagacatcagtcaagacagcgaagaagaaagagaagaggcgcagcttgtagcagtaggtttcagctacccgcccgtgcgcatcgttgagaaggatggcaaaagaagcattgaaaagattgccaaagatgatcctctggctaaaggagctgctgcctgtactgttaagaatccgattagcttgcccctggtgtctgcttgggagaagggcatggaagtgatgtgtgtacttatggagaagtacaggctggataacgaacttagaacctcttttaaactaatgcctgagcagcacgaacaatacaagcgcatctgtcaccagtacgtaaacgaggagcaccgaggcgtccagttaactttcactagtcacaagacactgagcacgatgatgggaagatttctgcagggaatgatacattctttttctcagatcgcccaccataattgggaatgtacaggctgtgcgttgtggcctcatggctgcaatgattatgagggcaagctgaagtgcctgcatggcactattatgattcagaaggagcagattatcgagatggatgtggccagcgagaatggacaacgagccttgaaggagaatcccgagcgaaccaaaatcacccaaaaccgctggggcagaagtgtggtgcaaatagccaacaatgacgcccgctgctgtgtgaatgatgccggctgcgcagctaaccagttttcgagcaggtcctgcgggatgttctataccgagggtagtaaagcccagcaggctttcaagcaatatgatgctttcatgagagccgtgtatccaggaatcagacaggatcaggccaagatgatactaatccccctgcactgcgattgcaaccacaagcctaattgggtgcccgccatgggtaggcagacctgcaagatgaccccgttcagcattgccaatgctgaagatctggatgtgggcatgattgcagatcccaccgtgctggcaagcgtgagacatccgtcgctgatggtgtttcagtgctgcaatccggtgtaccgcaactctcgcgctcagagcactggtcctaattgtgatttcaagatttcggctcctgacctgctgggagctttgcagctgaccagaaagctgtggagtgacatcctacccgatataccggttcccaagttggtgattcccgaattcaagtggcaacccaagtatcagttccgcaacgtatcactgcccgcgggtcattctgattctcgtcagaacccattcgacttgtaa

SEQ ID NO: 11 - DNA, 2016 bp, Human adenovirus type 5
atggccctgagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttag

SEQ ID NO: 12 - DNA, 483 bp, Human adenovirus type 5
atgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaa

(19) United States
(12) Patent Application Publication
(10) Pub. No.: US 2024/0165175 A1
(43) Pub. Date: May 23, 2024

(54) MUC16 PROMOTER CONTAINING VIRUS

(71) Applicant: City of Hope, Duarte, CA (US)

(72) Inventors: Edward Wang, Duarte, CA (US); Er Yue, Duarte, CA (US)

(21) Appl. No.: 18/279,581

(22) PCT Filed: Mar. 8, 2022
(86) PCT No.: PCT/US2022/019371

§ 371 Date: Aug. 30, 2023

Related U.S. Application Data
(60) Provisional application No. 63/158,858, filed on Mar. 9, 2021.
(60) Provisional application No. 63/235,379, filed on Aug. 20, 2021.

(51) Int. Cl. A61K 35/761; A61K 38/19; A61K 38/45; A61P 35/00; C07K 16/28

(52) U.S. Cl. CPC A61K 35/761; A61K 38/191; A61K 38/193; A61K 38/45; A61P 35/00; C07K 16/2818; C07K 16/2827

(57) ABSTRACT

Provided herein are, inter alia, viral compositions and methods of using the same. The viral compositions comprise a virus including a nucleic acid encoding a MUC16 promoter operably linked to an essential viral gene. The compositions provided herein are contemplated to be particularly useful for treating CA-125 expressing cancers.

### CROSS-REFERENCE TO RELATED APPLICATIONS

This application claims priority to U.S. Provisional Application No. 63/158,858, filed Mar. 9, 2021, and U.S. Provisional Application No. 63/235,379, filed Aug. 20, 2021, which are hereby incorporated by reference in their entirety and for all purposes.

### STATEMENT AS TO RIGHTS TO INVENTIONS MADE UNDER FEDERALLY SPONSORED RESEARCH AND DEVELOPMENT

This invention was made with government support under K12CA001727 and 1R21CA267570 awarded by the National Institutes of Health. The government has certain rights in the invention.

### REFERENCE TO A SEQUENCE LISTING, A TABLE OR A COMPUTER PROGRAM LISTING APPENDIX SUBMITTED AS AN ASCII TEXT FILE

The Sequence Listing written in file 048440-802001WO SequenceListing_ST25.TXT, created on Mar. 8, 2022, 20,480 bytes, machine format IBM-PC, MS Windows operating system, is hereby incorporated by reference.

### BACKGROUND

Despite modern advances, ovarian cancer remains one of the most common causes of cancer-related death of women in the US. Due to lack of an effective screening test, ovarian cancer typically presents at an advanced stage. Current treatment of ovarian cancer is primarily limited to surgery and chemotherapy, and the five-year survival rate is below 50%. New treatments are urgently needed to help patients suffering from this deadly disease. A unique feature of ovarian cancer is that more than 80% of patients express a high serum level of CA-125. Bioinformatic analysis shows that CA-125 mRNA is also highly expressed in gynecological cancer cells, with the highest in ovarian cancer cells, but not in most other cancer cells or in normal cells. CA-125 has been regarded as an ideal and unique target for ovarian cancer treatment; however, targeting CA-125 protein for ovarian cancer treatment has never been successful. Moreover, specific transcriptional activation of MUC16 (the gene encoding CA-125) in ovarian cancer cells is poorly defined.

CA-125-targeted antibodies, conjugated with or without radioactive isotopes or cytotoxic drugs, such as Oregovomab and DMUC5754A, have been developed but showed no clinical advantage in large randomized placebo-controlled trials (7-10).

Disclosed herein, inter alia, are solutions to these and other problems in the art.

### BRIEF SUMMARY OF THE INVENTION

Provided herein, inter alia, are virus compositions and methods of use thereof for targeting MUC16 expressing cells for the treatment of CA-125 expressing cancers. The viruses provided herein conditionally replicate in CA-125-expressing cells. Applicant demonstrates targeted treatment of CA-125-expressing cancers (e.g. ovarian cancer, etc.) with the virus provided herein, and protective anti-cancer immune response induced by virus-infected cancer cells.

In an aspect is provided a virus including a nucleic acid encoding a MUC16 promoter operably linked to an essential viral gene.

In an aspect is provided a method of forming a virus provided herein including embodiments thereof, the method including: i) contacting a cell with a nucleic acid encoding essential viral genes and the MUC16 promoter; and ii) allowing the cell to express the essential viral genes.

In another aspect is provided an isolated nucleic acid encoding a virus provided herein including embodiments thereof.

In an aspect a pharmaceutical composition is provided, the pharmaceutical composition including a therapeutically effective amount of a virus provided herein including embodiments thereof.

In an aspect a cell including the virus provided herein including embodiments thereof is provided.

In an aspect a method of treating or preventing cancer in a subject in need thereof is provided, the method including administering to the subject a therapeutically or prophylactically effective amount of a virus provided herein including embodiments thereof.

In an aspect is provided a method of stimulating an immune response in a subject in need thereof, the method including administering to the subject an effective amount of a virus provided herein including embodiments thereof.

In another aspect is provided method of treating or preventing cancer in a subject in need thereof, the method including administering to the subject a therapeutically or prophylactically effective amount of a cell including a virus provided herein including embodiments thereof.

In another aspect is provided a method of stimulating an immune response in a subject in need thereof, the method including administering to the subject an effective amount of a cell including the virus provided herein including embodiments thereof.

In an aspect a method of inhibiting proliferation of a CA-125 expressing cell is provided, the method including contacting the CA-125 expressing cell with a virus provided herein including embodiments thereof.

### BRIEF DESCRIPTION OF THE DRAWINGS

FIG. 1. shows CA-125 mRNA levels in human cancers.

FIG. 2. shows transcriptional activity of MUC16 promoters. Relative activity of MUC16 upstream fragment in HEK293 cells (CA-125-low) and HeLa cells (CA-125-high) using a pGL-2 reporter (left panel). Relative activity of MUC16 fragments in A549 cells (CA-125-low) and HeLa cells using the Ad5/E1A promoter (right panel). ***p=0.0002 pGL4.14-MUC16-full: HeLa vs. pGL4.14-MUC16-full: HEK293, ####p<0.0001 pGL4.14-MUC16-1040: HeLa vs. pGL4.14-MUC16-1040: HEK293, \hat{ }\hat{ }\hat{ }\hat{ }p<0.0001 pGL4.14-E1A: A549 vs. pGL4.14-MUC16-1040: A549, ns, no statistical significance, n=3.

FIG. 3. is an illustration of CRAd genome recombination. ElA promoter of Ad5 was replaced by the MUC16 1040 fragment. HSV-TK was inserted in the E3 region.

FIGS. 4A-4C. illustrates that Ad5/MUC16-1040/TKeGFP virus replicates and lyses cancer cells expressing CA-125. FIG. 4A. shows that the virus induces CPE of OVCAR3 cells. FIG. 4B. Relative MUC16 mRNA expression in selected cell lines. FIG. 4C. Oncolysis induced by Ad5/MUC16-1040/TK-EGFP in selected human ovarian cancer cell lines. Coomassie blue stain assay: Live cells attached to plate culture surface were stained blue. Dead cells floated and were washed out, leaving the culture surface blank and devoid of stain. Intensity of blue indicates cell density. Punctuated white areas in the SKOV3 cells were non-specific detachment of cells. MOI: multiplicity of infection, virus:cell infection ratio.

FIGS. 5A-5C. illustrates Ad5/MUC16-1040/TK-eGFP prolongs survival of MDAH2774-bearing NSG mice. Mice were injected intraperitoneally (ip) with MDAH2774/FL cells (5×10⁶/mouse) on day −5, virus (1×10⁸ pfu/mouse) or virus-infected cells (1×10⁵/mouse) were injected ip on day 0. FIG. 5A. shows representative images of mice with firefly luciferase (FL) signal on day 15. FIG. 5B. shows FL intensity of mice treated with the virus and virus infected cells. FIG. 5C illustrates survival of mice treated with the virus or virus-infected cells.

FIGS. 6A and 6B. shows that Ad5/MUC16-1040/mGM-CSF-infected cells induce protective anti-cancer immune response. FIG. 6A. shows survival of C57BL/6 mice immunized with 1×10⁵ ID8 cells infected with Ad5/MUC16 1040/GM-CSF subcutaneously (sc) on day −15 and day −7, rechallenged with 2×10⁶ parental ID8 cells ip on day 0. FIG. 6B. illustrates FBV/NJ mice immunized 1×10⁵ STOSE cells infected with Ad5/MUC16-1040/GM-CSF sc on day −15 and day −7, rechallenged with 2×10₆ parental STOSE cells ip on day 0. Bioluminescent imaging was captured on day 21.

FIG. 7. shows the sequence of a 5,033 base pair DNA fragment (SEQ ID NO:3) of the MUC16 gene (chr19: 8980639-8985671), including a region upstream from the transcription start site (TSS) and a region downstream from the TSS that contains both the 5′ untranslated region and the first 136 bp of the open reading frame. The DNA fragment includes the 1040 base pair promoter (SEQ ID NO: 1; ch19: 8981294:8982333) (underlined) and the 646 base pair promoter (SEQ ID NO:2; ch19: 8981688-8982333) (highlighted in dark gray). The portion including the MUC16 exome 1 (highlighted in light gray) includes the 5′UTR and part of the open reading frame (in capital letters).

FIG. 8. Illustration of MUC16 upstream fragments and construction of firefly luciferase reporter.

FIGS. 9A-9C. Generation of recombinant CRAd withMUC16 1040 bp upstream fragment to control ElA expression. FIG. 9A. Ad5/MUC16-1040/TK-EGFP was packaged using HEK293 cells and assessed for viral plague formation and EGFP expression. FIG. 9B. Oncolysis (CPE) of HeLa cells. FIG. 9C. GCV-induced cell death in the virus-infected mouse ovarian cancer line ID8 cells, ***p=0.0007 vs Ctrl, n=3.

FIG. 10. Replication of Ad5/MUC16-1040/TK-EGFP is dependent on CA-125 expression in a panel of selected ovarian cancer cell lines. ElA expression in selected ovarian cancer cell lines infected with Ad5/MUC16-1040/TK-EGFP.

FIGS. 11A and 11B. Ad5/MUC16-1040/TK-EGFP replicates in primary ovarian cancer cells. FIG. 11A. Case 1 from malignant pleural effusion. Upper two panels: Confirmation of high-grade serous ovarian cancer with expression of CA-125, PAX8, WT-1, and p53 (mutated pattern with abnormal strong diffuse expression), negative for calretinin; Lower two panels: Ad5/MUC16-1040/TK-EGFP replicates in primary ovarian cancer cells indicated by expression of EGFP signal. FIG. 11B. Case 2 from ascites. Upper panel: Confirmation of high-grade serous ovarian cancer with expression of CA-125 and PAX8, negative for p53 (null pattern); Lower two panels: predominant cancer cells with EGFP expression, indicating virus replication.

FIGS. 12A-12D. Oncolytic activity in ovarian cancer xenograft models using immunodeficient NSG mice. FIG. 12A. Representative mouse in each group of the NSG/Kuramochi model imaged on day 0 and day 50. FIG. 12B. Firefly luciferase intensity of NSG/Kuramochi groups on day 0 and day 50. ***p=0.0004, CRAd group vs control group, ns, no statistical significance, n=5. FIG. 12C. Survival curve of NSG/Ku-ramochi model. **p=0.0097, n=5. FIG. 12D. Survival curve of NSG/OVCAR4 xenograft model with n=5 in each group. *p=0.0204, control vs CRAd; **p=0.0028, control vs CRAd-infected cells, *p=0.0326, CRAd vs CRAd-infected cells.

FIG. 13. Illustration of oncolytic adenovirus construction with immunoregulatory genes.

FIG. 14. CRAd-infected cells induce protective anti-cancer responses. Top panel: Survival of C57BL/6 mice immunized sc with ID8 cells infected with Ad5/MUC16 carrying GM-CSF and CD137L when rechallenged ip with parental ID8 cells. Bottom panel: Survival of FBV/NJ mice immunized sc with STOSE cells infected with CRAd when rechallenged ip with 2×10⁶ parental STOSE cells.

FIGS. 15A-15D. Lymphocyte proliferation induced by Ad/MUC16/hIL-2 infection of primary cells from malignant ascites. FIG. 15A. Control, mostly primary ovarian cancer cells from the ascites with a few countable lymphocytes. FIG. 15B. Ad/MUC16/hIL-2 infection induced lymphocyte proliferation. GZMB-positive CD8 cells in FIG. 15C. primary cells from ascites and FIG. 15D. Ad/MUC16/hIL-2.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non-limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. For example, the nucleic acid provided herein may be part of an adenoviral vector, which may be transduced into a cell. Polynucleotides useful in the methods of the disclosure may comprise natural nucleic acid sequences and variants thereof, artificial nucleic acid sequences, or a combination of such sequences.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., CA-125) in other proteins with different numbering systems. For example, by performing a simple sequence alignment with a protein (e.g., CA-125) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three-dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “CA-125 protein” or “CA-125” as used herein includes any of the recombinant or naturally-occurring forms of CA-125 protein, also known as Mucin-16, MUC16, ovarian cancer-related tumor marker CA125, ovarian carcinoma antigen CA125, or variants or homologs thereof that maintain CA-125 activity (e.g. within at least 50%, 80%, 90%, 95%, 96%, 97%, 98%, 99% or 100% activity compared to CA-125). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CA-125 protein. In embodiments, the CA-125 protein is substantially identical to the protein identified by the UniProt reference number Q8WXI7 or a variant or homolog having substantial identity thereto.

The term “GM-CSF protein” or “GM-CSF” as used herein includes any of the recombinant or naturally-occurring forms of granulocyte-macrophage colony-stimulating factor protein, also known as Colony-stimulating factor, CSF, Molgramostin, or variants or homologs thereof that maintain GM-CSF activity (e.g. within at least 50%, 80%, 90%, 95%, 96%, 97%, 98%, 99% or 100% activity compared to GM-CSF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GM-CSF protein. In embodiments, the GM-CSF protein is substantially identical to the protein identified by the UniProt reference number P04141 or a variant or homolog having substantial identity thereto.

The term “M-CSF protein” or “M-CSF” as used herein includes any of the recombinant or naturally-occurring forms of macrophage colony-stimulating factor protein, also known as CSF-1, MCSF or variants or homologs thereof that maintain M-CSF activity (e.g. within at least 50%, 80%, 90%, 95%, 96%, 97%, 98%, 99% or 100% activity compared to M-CSF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring M-CSF protein. In embodiments, the M-CSF protein is substantially identical to the protein identified by the UniProt reference number P09603 or a variant or homolog having substantial identity thereto.

The term “G-CSF protein” or “G-CSF” as used herein includes any of the recombinant or naturally-occurring forms of Granulocyte colony-stimulating factor receptor, also known as G-CSF receptor, CD114, or variants or homologs thereof that maintain G-CSF activity (e.g. within at least 50%, 80%, 90%, 95%, 96%, 97%, 98%, 99% or 100% activity compared to G-CSF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring G-CSF protein. In embodiments, the G-CSF protein is substantially identical to the protein identified by the UniProt reference number Q99062 or a variant or homolog having substantial identity thereto.

The term “IL-2 protein” or “IL-2” as used herein includes any of the recombinant or naturally-occurring forms of interleukin-2 protein, also known as T-cell growth factor, TCGF, Aldesleukin, or variants or homologs thereof that maintain IL-2 activity (e.g. within at least 50%, 80%, 90%, 95%, 96%, 97%, 98%, 99% or 100% activity compared to IL-2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 protein. In embodiments, the IL-2 protein is substantially identical to the protein identified by the UniProt reference number P60568 or a variant or homolog having substantial identity thereto.

The term “IL-12B protein” or “IL-12B” as used herein includes any of the recombinant or naturally-occurring forms of Interleukin-12 subunit beta protein, also known as Cytotoxic lymphocyte maturation factor 40 kDa subunit, CLMF p40, NK cell stimulatory factor chain 2, or variants or homologs thereof that maintain IL-12B activity (e.g. within at least 50%, 80%, 90%, 95%, 96%, 97%, 98%, 99% or 100% activity compared to IL-12B). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-12B protein. In embodiments, the IL-12B protein is substantially identical to the protein identified by the UniProt reference number P29460 or a variant or homolog having substantial identity thereto.

The term “IL-12A protein” or “IL-12A” as used herein includes any of the recombinant or naturally-occurring forms of Interleukin-12 subunit alpha, also known as Cytotoxic lymphocyte maturation factor 35 kDa subunit, CLMF p35, IL-12 subunit p35, NK cell stimulatory factor chain 1, NKSF1, or variants or homologs thereof that maintain IL-12A activity (e.g. within at least 50%, 80%, 90%, 95%, 96%, 97%, 98%, 99% or 100% activity compared to IL-12A). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-12A protein. In embodiments, the IL-12A protein is substantially identical to the protein identified by the UniProt reference number P29459 or a variant or homolog having substantial identity thereto.

The term “B7-1 protein” or “B7-1” as used herein includes any of the recombinant or naturally-occurring forms of B7-1 protein, also known as T-lymphocyte activation antigen CD80, CD80, Activation B7-1 antigen, BB1, CTLA-4 counter-receptor B7.1, B7, or variants or homologs thereof that maintain B7-1 activity (e.g. within at least 50%, 80%, 90%, 95%, 96%, 97%, 98%, 99% or 100% activity compared to B7-1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring B7-1 protein. In embodiments, the B7-1 protein is substantially identical to the protein identified by the UniProt reference number P33681 or a variant or homolog having substantial identity thereto.

The term “4-1BBL protein” or “4-1BBL” as used herein includes any of the recombinant or naturally-occurring forms of 4-1BBL protein, also known as Tumor necrosis factor ligand superfamily member 9, 4-1BB ligand, or variants or homologs thereof that maintain 4-1BBL activity (e.g. within at least 50%, 80%, 90%, 95%, 96%, 97%, 98%, 99% or 100% activity compared to 4-1BBL). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring 4-1BBL protein. In embodiments, the 4-1BBL protein is substantially identical to the protein identified by the UniProt reference number P41273 or a variant or homolog having substantial identity thereto.

The term “IL-15 protein” or “IL-15” as used herein includes any of the recombinant or naturally-occurring forms of interleukin-15, or variants or homologs thereof that maintain IL-15 activity (e.g. within at least 50%, 80%, 90%, 95%, 96%, 97%, 98%, 99% or 100% activity compared to IL-15). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 protein. In embodiments, the IL-15 protein is substantially identical to the protein identified by the UniProt reference number P40933 or a variant or homolog having substantial identity thereto.

The term “CD25 protein” or “CD25” as used herein includes any of the recombinant or naturally-occurring forms of CD25 protein, also known as interleukin-2 receptor subunit alpha, IL-2-RA, TAC antigen, p55, or variants or homologs thereof that maintain CD25 activity (e.g. within at least 50%, 80%, 90%, 95%, 96%, 97%, 98%, 99% or 100% activity compared to CD25). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD25 protein. In embodiments, the CD25 protein is substantially identical to the protein identified by the UniProt reference number P01589 or a variant or homolog having substantial identity thereto.

The term “TRAIL protein” or “TRAIL” as used herein includes any of the recombinant or naturally-occurring forms of TRAIL protein, also known as TNF-related apoptosis-inducing ligand, tumor necrosis factor ligand superfamily member 10, Apo-2 ligand, CD253, or variants or homologs thereof that maintain TRAIL activity (e.g. within at least 50%, 80%, 90%, 95%, 96%, 97%, 98%, 99% or 100% activity compared to TRAIL). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TRAIL protein. In embodiments, the TRAIL protein is substantially identical to the protein identified by the UniProt reference number P50591 or a variant or homolog having substantial identity thereto.

A “PD-1 protein” or “PD-1” as referred to herein includes any of the recombinant or naturally-occurring forms of the Programmed cell death protein 1 (PD-1) also known as cluster of differentiation 279 (CD 279) or variants or homologs thereof that maintain PD-1 protein activity (e.g. within at least 50%, 80%, 90%, 95%, 96%, 97%, 98%, 99% or 100% activity compared to PD-1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 protein. In embodiments, the PD-1 protein is substantially identical to the protein identified by the UniProt reference number Q15116 or a variant or homolog having substantial identity thereto. In embodiments, the PD-1 protein is substantially identical to the protein identified by the UniProt reference number Q02242 or a variant or homolog having substantial identity thereto.

A “PD-L1” or “PD-L1 protein” as referred to herein includes any of the recombinant or naturally-occurring forms of programmed death ligand 1 (PD-L1) also known as cluster of differentiation 274 (CD 274) or variants or homologs thereof that maintain PD-L1 activity (e.g. within at least 50%, 80%, 90%, 95%, 96%, 97%, 98%, 99% or 100% activity compared to PD-L1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto.

The term “CTLA-4” or “CTLA-4 protein” as provided herein includes any of the recombinant or naturally-occurring forms of the cytotoxic T-lymphocyte-associated protein 4 (CTLA-4) or variants or homologs thereof that maintain CTLA-4 protein activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 polypeptide. In embodiments, the CTLA-4 protein is substantially identical to the protein identified by the UniProt reference number P16410 or a variant or homolog having substantial identity thereto.

The terms “MUC16 promoter” as used herein refer to the any of the recombinant forms or fragments of the MUC16 promoter or variants or homologs thereof that maintain the activity of the MUC16 promoter (e.g., within at least 40%, 50%, 60%, 70%, 80%, 90%, 95%, 96%, 97%, 98%, 99% or 100% activity compared to the MUC16 promoter). In some aspects, the variants or homologs have at least 70%, 80%, 90%, 95%, 96%, 97%, 98%, 99% or 100% nucleic acid sequence identity across the whole sequence or a portion of the sequence (e.g., a 20, 50, 100, 150 or 200 continuous nucleic acid portion) compared to a naturally occurring MUC16 promoter. In embodiments, the MUC16 promoter is substantially identical to a portion or a fragment of the nucleic acid sequence corresponding to position 29,720 to 34,752 of the nucleic acid sequence identified by Accession No. NG_055257.1. In embodiments, the MUC16 promoter is substantially identical to a portion of the nucleic acid sequence corresponding to position 29,720 to 34,752 of the nucleic acid sequence identified by Accession No. NG_055257.1. In embodiments, the MUC16 promoter includes the nucleic acid sequence of SEQ ID NO:1. In embodiments, the MUC16 promoter is the nucleic acid sequence of SEQ ID NO:1. In embodiments, the MUC16 promoter includes a fragment or portion of the nucleic acid sequence of SEQ ID NO:1. In embodiments, the MUC16 promoter includes the nucleic acid sequence of SEQ ID NO:2. In embodiments, the MUC16 promoter is the nucleic acid sequence of SEQ ID NO:2. In embodiments, the WUC16 promoter includes a fragment or portion of the nucleic acid sequence of SEQ ID NO:2. In embodiments, the MUC16 promoter includes the nucleic acid sequence of SEQ ID NO:3. In embodiments, the MUC16 promoter is the nucleic acid sequence of SEQ ID NO:3. In embodiments, the MUC16 promoter includes a fragment or portion of the nucleic acid sequence of SEQ ID NO:3.

The terms “E1A gene”, “control protein E1A gene”, or the like, as used herein refer to the any of the recombinant or naturally-occurring forms of the E1A gene or variants or homologs thereof that code for a E1A polypeptide capable of maintaining the activity of the E1A polypeptide (e.g., within at least 50%, 80%, 90%, 95%, 96%, 97%, 98%, 99% or 100% activity compared to E1A). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring E1A. In embodiments, the E1A gene is substantially identical to the nucleic acid sequence corresponding to position 467 to 1,630 of the nucleic acid sequence identified by Accession No. NC_001405.1 or a variant or homolog having substantial identity thereto. In embodiments, the E1A gene includes the nucleic acid sequence of SEQ ID NO:8. In embodiments, the E1A gene is the nucleic acid sequence of SEQ ID NO:8.

The terms “E1B gene”, “control protein E1B gene”, or the like, as used herein refer to the any of the recombinant or naturally-occurring forms of the E1B gene or variants or homologs thereof that code for a E1B polypeptide capable of maintaining the activity of the E1B polypeptide (e.g., within at least 50%, 80%, 90%, 95%, 96%, 97%, 98%, 99% or 100% activity compared to E1B). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring E1B. In embodiments, the E1B gene is substantially identical to the nucleic acid sequence corresponding to position 1,669-4061 of the nucleic acid sequence identified by Accession No. NC_001405.1 or a variant or homolog having substantial identity thereto. In embodiments, the E1B gene includes the nucleic acid sequence of SEQ ID NO:9. In embodiments, the E1B gene is the nucleic acid sequence of SEQ ID NO:9.

The terms “E2A gene”, “single-stranded DNA-binding protein gene”, or the like, as used herein refer to the any of the recombinant or naturally-occurring forms of the E2A gene or variants or homologs thereof that code for a E2A polypeptide capable of maintaining the activity of the E2A polypeptide (e.g., within at least 50%, 80%, 90%, 95%, 96%, 97%, 98%, 99% or 100% activity compared to E2A). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring E2A. In embodiments, the E2A gene is substantially identical to the nucleic acid sequence corresponding to position 21,178-25,523 of the nucleic acid sequence identified by Accession No. NC_001460.1 or a variant or homolog having substantial identity thereto. In embodiments, the E2A gene includes the nucleic acid sequence of SEQ ID NO:10. In embodiments, the E2A gene is the nucleic acid sequence of SEQ ID NO:10.

The terms “E2B gene”, “DNA polymerase gene”, or the like, as used herein refer to the any of the recombinant or naturally-occurring forms of the E2B gene or variants or homologs thereof that code for a E2B polypeptide capable of maintaining the activity of the E2B polypeptide (e.g., within at least 50%, 80%, 90%, 95%, 96%, 97%, 98%, 99% or 100% activity compared to E2B). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring E2B. In embodiments, the E2B gene is substantially identical to the nucleic acid sequence corresponding to position 3,963-26,445 of the nucleic acid sequence identified by Accession No. NC_001202.1 or a variant or homolog having substantial identity thereto. In embodiments, the E2B gene includes the nucleic acid sequence of SEQ ID NO:11. In embodiments, the E2B gene is the nucleic acid sequence of SEQ ID NO:11.

The terms “E3 gene”, “early region 3 protein gene”, or the like, as used herein refer to the any of the recombinant or naturally-occurring forms of the E3 gene or variants or homologs thereof that code for a E3 polypeptide capable of maintaining the activity of the E3 polypeptide (e.g., within at least 50%, 80%, 90%, 95%, 96%, 97%, 98%, 99% or 100% activity compared to E3). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring E3. In embodiments, the E3 gene is substantially identical to the nucleic acid sequence corresponding to position 26,686-31,469 of the nucleic acid sequence identified by Accession No. NC_003266.2 or a variant or homolog having substantial identity thereto. In embodiments, the E3 gene includes the nucleic acid sequence of SEQ ID NO:12. In embodiments, the E3 gene is the nucleic acid sequence of SEQ ID NO:12.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Nucleic acid is “operably linked” when it is placed into a functional relationship with another nucleic acid sequence (e.g. a promoter sequence). For example, a promoter (e.g. MUC16 promoter) or enhancer is operably linked to a coding sequence if it affects the transcription of the sequence; or a ribosome binding site is operably linked to a coding sequence if it is positioned so as to facilitate translation. Operably linked means that the nucleotide sequences being linked are typically contiguous. However, as enhancers generally function when separated from the promoter by several kilobases and intronic sequences may be of variable lengths, some polynucleotide elements may be operably linked but not directly flanked and may even function in trans from a different allele or chromosome. Linking may be accomplished by ligation at convenient sites. If such sites do not exist, the synthetic oligonucleotide adaptors or linkers are used in accordance with conventional practice.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The term “recombinant” when used with reference, e.g., to a virus, cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, a recombinant virus is generated by combining portions of nucleic acids using recombinant nucleic acid technology. For example, a recombinant virus may be generated by replacing one or more viral genes with an exogenous gene. For example, a recombinant virus may be generated by replacing a viral promoter with an exogenous promoter (e.g. MUC16 promoter). Thus, in embodiments, the virus provided herein including embodiments thereof is a recombinant virus. In instances, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. In embodiments, the MUC16 promoter provided herein including embodiments thereof is exogenous to the virus. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A nucleic acid that is the predominant species present in a preparation is substantially purified.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector (e.g. adenovirus vector) may be used in the methods described herein. Examples for viral vectors include, but are not limited to retroviral, adenoviral, lentiviral and adeno-associated viral vectors. In some embodiments, the nucleic acid molecules are introduced into a cell using an adenoviral vector following standard procedures well known in the art. The terms “transfection” or “transduction” also refer to introducing proteins into a cell from the external environment. In embodiments, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Transduce” or “transduction” are used according to their plain ordinary meanings and refer to the process by which one or more foreign nucleic acids (i.e. DNA not naturally found in the cell) are introduced into a cell. Typically, transduction occurs by introduction of a virus or viral vector (e.g. adenovirus vector) into the cell. For example, an adenovirus vector including a MUC16 promoter operably linked to an essential viral gene may be transduced into a cell, thereby allowing expression of the essential viral gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, an virus as described herein and a CA-125 expressing cancer cell. In embodiments contacting includes, for example, allowing a virus as described herein to physically touch a CA-125 expressing cancer cell.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. In embodiments, the cell is a human cell. In embodiments, the cell is a CA-125 cancer expressing cell. Cells may be useful when they are naturally nonadherent or have been treated not to adhere to surfaces, for example by trypsinization.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus, poxvirus), an envelope including lipids and optionally components of host cell membranes, and/or viral proteins.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. Thus, the term “replicate” includes passaging and re-infecting progeny cells. In the context of a virus, the term “replicate” includes the ability of a virus to replicate (duplicate the viral genome and packaging said genome into viral particles) in a host cell and subsequently release progeny viruses from the host cell, which results in the lysis of the host cell.

The term “plaque forming units” is used according to its plain ordinary meaning in Virology and refers to the amount of plaques in a cell monolayer that can be formed per volume of viral particles. In some embodiments the units are based on the number of plaques that could form when infecting a monolayer of susceptible cells. For example, in embodiments 1,000 PFU/μl indicates that 1 μl of a solution including viral particles contains enough virus particles to produce 1000 infectious plaques in a cell monolayer. In embodiments, plaque forming units are abbreviated “PFU”.

The terms “multiplicity of infection” or “MOI” are used according to its plain ordinary meaning in Virology and refers to the ratio of infectious agent (e.g., poxvirus) to the target (e.g., cell) in a given area or volume. In embodiments, the area or volume is assumed to be homogenous.”:

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). In embodiments, “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

The terms “disease” or “condition” refer to a state of being or health status of a patient or subject capable of being treated with the compounds or methods provided herein. The disease may be a cancer. In some further instances, “cancer” refers to human cancers, including gynecological cancers (e.g. ovarian cancer, endometrial cancer, uterine cancer, etc.). In embodiments, the cancer is a CA-125 expressing cancer.

The term “associated” or “associated with” in the context of a substance or substance activity or function associated with a disease (e.g. ovarian cancer, a CA-125 expressing cancer) is caused by (in whole or in part), or a symptom of the disease is caused by (in whole or in part) the substance or substance activity or function.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

“Patient” or “subject in need thereof” refers to a living organism suffering from or prone to a disease (e.g. cancer, a CA-125 expressing cancer, ovarian cancer, etc.) or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the term “cancer” refers to all types of cancer, neoplasm or malignant tumors found in mammals (e.g., humans), including gynecological cancers. Exemplary cancers that may be treated with a compound or method provided herein include ovarian cancer, endometrial cancer, and uterine cancer. In embodiments, the cancer is ovarian cancer. In embodiments, the cancer is endometrial cancer. In embodiments, the cancer is uterine cancer. In embodiments, the cancer is a CA-125 expressing cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

For prophylactic use, a therapeutically effective amount of the virus composition described herein are administered to a subject prior to or during early onset (e.g., upon initial signs and symptoms of cancer). Therapeutic treatment involves administering to a subject a therapeutically effective amount of the agents described herein after diagnosis or development of disease.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” is used in accordance with its plain and ordinary meaning and includes oral administration, administration as a suppository, topical contact, intravenous, intraperitoneal, intrapleural, intramuscular, intralesional, intratumoral, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

### Viral Compositions

Provided herein, inter alia, is a conditionally replicating virus including a nucleic acid encoding the MUC16 promoter. The MUC16 promoter is operably linked to an essential viral gene; therefore, the virus provided herein including embodiments thereof selectively infects and replicates in MUC16 expressing cells. Thus, in an aspect is provided a virus including a nucleic acid encoding a MUC16 promoter operably linked to an essential viral gene. As used herein, “MUC16 promoter” refers to a nucleic acid sequence including a fragment of the naturally occurring MUC16 promoter. In embodiments, the MUC16 promoter includes a nucleic acid sequence substantially identical to a region from approximately position 29,720 to 34,752 of the nucleic acid sequence identified by Accession No. NG_055257.1. In embodiments, the MUC16 promoter can include a nucleic acid sequence upstream from a MUC16 transcription start site (TSS). In embodiments, the MUC16 promoter can include a nucleic acid sequence downstream from a MUC16 TSS. In instances, the MUC16 promoter can include a 5′untranslated region (5′UTR) of a MUC16 gene. For example, the MUC16 promoter can be a fragment of the nucleic acid sequence of SEQ ID NO:3. In embodiments, the MUC16 promoter includes the nucleic acid sequence of SEQ ID NO:1 or 2 or a fragment of the nucleic acid sequence of SEQ ID NO:1 or 2.

In embodiments, the MUC16 promoter is from about 20 to about 6000 nucleic acid residues in length. In embodiments, said MUC16 promoter is from about 50 to about 6000 nucleic acid residues in length. In embodiments, the MUC16 promoter is from about 200 to about 6000 nucleic acid residues in length. In embodiments, the MUC16 promoter is from about 350 to about 6000 nucleic acid residues in length. In embodiments, the MUC16 promoter is from about 500 to about 6000 nucleic acid residues in length. In embodiments, the MUC16 promoter is from about 650 to about 6000 nucleic acid residues in length. In embodiments, the MUC16 promoter is from about 800 to about 6000 nucleic acid residues in length. In embodiments, the MUC16 promoter is from about 950 to about 6000 nucleic acid residues in length. In embodiments, the MUC16 promoter is from about 1100 to about 6000 nucleic acid residues in length. In embodiments, the MUC16 promoter is from about 1,250 to about 6000 nucleic acid residues in length. In embodiments, the MUC16 promoter is from about 1,400 to about 6000 nucleic acid residues in length. In embodiments, the MUC16 promoter is from about 1,550 to about 6000 nucleic acid residues in length. In embodiments, the MUC16 promoter is from about 1,700 to about 6000 nucleic acid residues in length. In embodiments, the MUC16 promoter is from about 1,850 to about 6000 nucleic acid residues in length. In embodiments, the MUC16 promoter is from about 2,000 to about 6000 nucleic acid residues in length. In embodiments, the MUC16 promoter is from about 2,150 to about 6000 nucleic acid residues in length. In embodiments, the MUC16 promoter is from about 2,300 to about 6000 nucleic acid residues in length. In embodiments, the MUC16 promoter is from about 2,450 to about 6000 nucleic acid residues in length. In embodiments, the MUC16 promoter is from about 2,600 to about 6000 nucleic acid residues in length. In embodiments, the MUC16 promoter is from about 2,750 to about 6000 nucleic acid residues in length. In embodiments, the MUC16 promoter is from about 2,900 to about 6000 nucleic acid residues in length. In embodiments, the MUC16 promoter is from about 3,050 to about 6000 nucleic acid residues in length. In embodiments, the MUC16 promoter is from about 3,200 to about 6000 nucleic acid residues in length. In embodiments, the MUC16 promoter is from about 3,350 to about 6000 nucleic acid residues in length. In embodiments, the MUC16 promoter is from about 3,500 to about 6000 nucleic acid residues in length. In embodiments, the MUC16 promoter is from about 3,650 to about 6000 nucleic acid residues in length. In embodiments, the MUC16 promoter is from about 3,800 to about 6000 nucleic acid residues in length. In embodiments, the MUC16 promoter is from about 3,950 to about 6000 nucleic acid residues in length. In embodiments, the MUC16 promoter is from about 4,100 to about 6000 nucleic acid residues in length. In embodiments, the MUC16 promoter is from about 4,250 to about 6000 nucleic acid residues in length. In embodiments, the MUC16 promoter is from about 4,400 to about 6000 nucleic acid residues in length. In embodiments, the MUC16 promoter is from about 4,550 to about 6000 nucleic acid residues in length. In embodiments, the MUC16 promoter is from about 4,700 to about 6000 nucleic acid residues in length. In embodiments, the MUC16 promoter is from about 4,850 to about 6000 nucleic acid residues in length. In embodiments, the MUC16 promoter is from about 5,000 to about 6000 nucleic acid residues in length. In embodiments, the MUC16 promoter is from about 5,150 to about 6000 nucleic acid residues in length. In embodiments, the MUC16 promoter is from about 5,300 to about 6000 nucleic acid residues in length. In embodiments, the MUC16 promoter is from about 5,450 to about 6000 nucleic acid residues in length. In embodiments, the MUC16 promoter is from about 5,600 to about 6000 nucleic acid residues in length. In embodiments, the MUC16 promoter is from about 5,750 to about 6000 nucleic acid residues in length. In embodiments, the MUC16 promoter is from about 5,900 to about 6000 nucleic acid residues in length.

In embodiments, the MUC16 promoter is from about 20 to about 5,900 nucleic acid residues in length. In embodiments, the MUC16 promoter is from about 20 to about 5,750 nucleic acid residues in length. In embodiments, the MUC16 promoter is from about 20 to about 5,600 nucleic acid residues in length. In embodiments, the MUC16 promoter is from about 20 to about 5,450 nucleic acid residues in length. In embodiments, the MUC16 promoter is from about 20 to about 5,300 nucleic acid residues in length. In embodiments, the MUC16 promoter is from about 20 to about 5,150 nucleic acid residues in length. In embodiments, the MUC16 promoter is from about 20 to about 5,000 nucleic acid residues in length. In embodiments, the MUC16 promoter is from about 20 to about 4,850 nucleic acid residues in length. In embodiments, the MUC16 promoter is from about 20 to about 4,700 nucleic acid residues in length. In embodiments, the MUC16 promoter is from about 20 to about 4,550 nucleic acid residues in length. In embodiments, the MUC16 promoter is from about 20 to about 4,400 nucleic acid residues in length. In embodiments, the MUC16 promoter is from about 20 to about 4,250 nucleic acid residues in length. In embodiments, the MUC16 promoter is from about 20 to about 4,100 nucleic acid residues in length. In embodiments, the MUC16 promoter is from about 20 to about 3,950 nucleic acid residues in length. In embodiments, the MUC16 promoter is from about 20 to about 3,800 nucleic acid residues in length. In embodiments, the MUC16 promoter is from about 20 to about 3,650 nucleic acid residues in length. In embodiments, the MUC16 promoter is from about 20 to about 3,400 nucleic acid residues in length. In embodiments, the MUC16 promoter is from about 20 to about 3,250 nucleic acid residues in length. In embodiments, the MUC16 promoter is from about 20 to about 3,100 nucleic acid residues in length. In embodiments, the MUC16 promoter is from about 20 to about 2,950 nucleic acid residues in length. In embodiments, the MUC16 promoter is from about 20 to about 2,700 nucleic acid residues in length. In embodiments, the MUC16 promoter is from about 20 to about 2,550 nucleic acid residues in length. In embodiments, the MUC16 promoter is from about 20 to about 2,400 nucleic acid residues in length. In embodiments, the MUC16 promoter is from about 20 to about 2,250 nucleic acid residues in length. In embodiments, the MUC16 promoter is from about 20 to about 2,100 nucleic acid residues in length. In embodiments, the MUC16 promoter is from about 20 to about 1,850 nucleic acid residues in length. In embodiments, the MUC16 promoter is from about 20 to about 1,700 nucleic acid residues in length. In embodiments, the MUC16 promoter is from about 20 to about 1,550 nucleic acid residues in length. In embodiments, the MUC16 promoter is from about 20 to about 1,400 nucleic acid residues in length. In embodiments, the MUC16 promoter is from about 20 to about 1,250 nucleic acid residues in length. In embodiments, the MUC16 promoter is from about 20 to about 1,100 nucleic acid residues in length. In embodiments, the MUC16 promoter is from about 20 to about 950 nucleic acid residues in length. In embodiments, the MUC16 promoter is from about 20 to about 800 nucleic acid residues in length. In embodiments, the MUC16 promoter is from about 20 to about 650 nucleic acid residues in length. In embodiments, the MUC16 promoter is from about 20 to about 500 nucleic acid residues in length. In embodiments, the MUC16 promoter is from about 20 to about 350 nucleic acid residues in length. In embodiments, the MUC16 promoter is from about 20 to about 200 nucleic acid residues in length. In embodiments, the MUC16 promoter is from about 20 to about 50 nucleic acid residues in length. In embodiments, the MUC16 promoter is about 20 nucleic acid residues, 50 nucleic acid residues, 200 nucleic acid residues, 350 nucleic acid residues, 500 nucleic acid residues, 650 nucleic acid residues, 800 nucleic acid residues, 950 nucleic acid residues, 1100 nucleic acid residues, 1250 nucleic acid residues, 1400 nucleic acid residues, 1550 nucleic acid residues, 1700 nucleic acid residues, 1850 nucleic acid residues, 2000 nucleic acid residues, 2150 nucleic acid residues, 2300 nucleic acid residues, 2450 nucleic acid residues, 2600 nucleic acid residues, 2750 nucleic acid residues, 2900 nucleic acid residues, 3050 nucleic acid residues, 3200 nucleic acid residues, 2350 nucleic acid residues, 3500 nucleic acid residues, 3650 nucleic acid residues, 3800 nucleic acid residues, 3950 nucleic acid residues, 4100 nucleic acid residues, 4250 nucleic acid residues, 4400 nucleic acid residues, 4550 nucleic acid residues, 4700 nucleic acid residues, 4850 nucleic acid residues, 5000 nucleic acid residues, 5150 nucleic acid residues, 5300 nucleic acid residues, 5450 nucleic acid residues, 5600 nucleic acid residues, 3750 nucleic acid residues, 5900 nucleic acid residues, or 6000 nucleic acid residues in length. In embodiments, the sequence lengths described herein are within nucleic acid sequence of SEQ ID NO:1. In embodiments, the sequence lengths described herein include the nucleic acid sequence of SEQ ID NO:1. In embodiments, the sequence lengths described herein include a fragment or a portion of the nucleic acid sequence of SEQ ID NO:1. In embodiments, the sequence lengths described herein are within nucleic acid sequence of SEQ ID NO:2. In embodiments, the sequence lengths described herein include the nucleic acid sequence of SEQ ID NO:2. In embodiments, the sequence lengths described herein include a fragment or a portion of the nucleic acid sequence of SEQ ID NO:2. In embodiments, the sequence lengths described herein are within nucleic acid sequence of SEQ ID NO:3. In embodiments, the sequence lengths described herein include the nucleic acid sequence of SEQ ID NO:3. In embodiments, the sequence lengths described herein include a fragment or a portion of the nucleic acid sequence of SEQ ID NO:3.

In embodiments, the MUC16 promoter is from about 800 nucleic acid residues to about 1200 nucleic acid residues in length. In embodiments, the MUC16 promoter is from about 850 nucleic acid residues to about 1200 nucleic acid residues in length. In embodiments, the MUC16 promoter is from about 900 nucleic acid residues to about 1200 nucleic acid residues in length. In embodiments, the MUC16 promoter is from about 950 nucleic acid residues to about 1200 nucleic acid residues in length. In embodiments, the MUC16 promoter is from about 1000 nucleic acid residues to about 1200 nucleic acid residues in length. In embodiments, the MUC16 promoter is from about 1050 nucleic acid residues to about 1200 nucleic acid residues in length. In embodiments, the MUC16 promoter is from about 1100 nucleic acid residues to about 1200 nucleic acid residues in length. In embodiments, the MUC16 promoter is from about 1150 nucleic acid residues to about 1200 nucleic acid residues in length.

In embodiments, the MUC16 promoter is from about 800 nucleic acid residues to about 1150 nucleic acid residues in length. In embodiments, the MUC16 promoter is from about 800 nucleic acid residues to about 1100 nucleic acid residues in length. In embodiments, the MUC16 promoter is from about 800 nucleic acid residues to about 1050 nucleic acid residues in length. In embodiments, the MUC16 promoter is from about 800 nucleic acid residues to about 1000 nucleic acid residues in length. In embodiments, the MUC16 promoter is from about 800 nucleic acid residues to about 950 nucleic acid residues in length. In embodiments, the MUC16 promoter is from about 800 nucleic acid residues to about 900 nucleic acid residues in length. In embodiments, the MUC16 promoter is from about 800 nucleic acid residues to about 850 nucleic acid residues in length. In embodiments, the MUC16 promoter is about 800 nucleic acid residues, 850 nucleic acid residues, 900 nucleic acid residues, 950 nucleic acid residues, 1000 nucleic acid residues, 1050 nucleic acid residues, 1100 nucleic acid residues, 1150 nucleic acid residues, or 1200 nucleic acid residues in length. In embodiments, the sequence lengths described herein are within nucleic acid sequence of SEQ ID NO:1. In embodiments, the sequence lengths described herein include the nucleic acid sequence of SEQ ID NO:1. In embodiments, the sequence lengths described herein include a fragment or a portion of the nucleic acid sequence of SEQ ID NO:1. In embodiments, the sequence lengths described herein include the nucleic acid sequence of SEQ ID NO:2. In embodiments, the sequence lengths described herein include a fragment or a portion of the nucleic acid sequence of SEQ ID NO:2. In embodiments, the sequence lengths described herein are within nucleic acid sequence of SEQ ID NO:3. In embodiments, the sequence lengths described herein include a fragment or a portion of the nucleic acid sequence of SEQ ID NO:3.

In embodiments, the MUC16 promoter is from about 400 to about 800 nucleic acid residues in length. In embodiments, the MUC16 promoter is from about 450 to about 800 nucleic acid residues in length. In embodiments, the MUC16 promoter is from about 500 to about 800 nucleic acid residues in length. In embodiments, the MUC16 promoter is from about 550 to about 800 nucleic acid residues in length. In embodiments, the MUC16 promoter is from about 600 to about 800 nucleic acid residues in length. In embodiments, the MUC16 promoter is from about 650 to about 800 nucleic acid residues in length. In embodiments, the MUC16 promoter is from about 700 to about 800 nucleic acid residues in length. In embodiments, the MUC16 promoter is from about 750 to about 800 nucleic acid residues in length.

In embodiments, the MUC16 promoter is from about 400 to about 750 nucleic acid residues in length. In embodiments, the MUC16 promoter is from about 400 to about 700 nucleic acid residues in length. In embodiments, the MUC16 promoter is from about 400 to about 650 nucleic acid residues in length. In embodiments, the MUC16 promoter is from about 400 to about 600 nucleic acid residues in length. In embodiments, the MUC16 promoter is from about 400 to about 550 nucleic acid residues in length. In embodiments, the MUC16 promoter is from about 400 to about 500 nucleic acid residues in length. In embodiments, the MUC16 promoter is from about 400 to about 450 nucleic acid residues in length. In embodiments, the MUC16 promoter is about 400 nucleic acid residues, 450 nucleic acid residues, 500 nucleic acid residues, 550 nucleic acid residues, 600 nucleic acid residues, 650 nucleic acid residues, 700 nucleic acid residues, 750 nucleic acid residues, or 800 nucleic acid residues in length. In embodiments, the sequence lengths described herein are within nucleic acid sequence of SEQ ID NO:1. In embodiments, the sequence lengths described herein include a fragment or a portion of the nucleic acid sequence of SEQ ID NO:1. In embodiments, the sequence lengths described herein are within nucleic acid sequence of SEQ ID NO:2. In embodiments, the sequence lengths described herein include the nucleic acid sequence of SEQ ID NO:2. In embodiments, the sequence lengths described herein include a fragment or a portion of the nucleic acid sequence of SEQ ID NO:2. In embodiments, the sequence lengths described herein are within nucleic acid sequence of SEQ ID NO:3. In embodiments, the sequence lengths described herein include a fragment or a portion of the nucleic acid sequence of SEQ ID NO:3.

In embodiments, the MUC16 promoter includes a nucleic acid sequence within a region from about −6000 nucleic acid residues upstream to about +200 nucleic residues downstream of a MUC16 transcription start site (TSS). In embodiments, the MUC16 promoter includes a nucleic acid sequence within a region from about −5800 nucleic acid residues upstream to about +200 nucleic residues downstream of a MUC16 TSS. In embodiments, the MUC16 promoter includes a nucleic acid sequence within a region from about −5600 nucleic acid residues upstream to about +200 nucleic residues downstream of a MUC16 TSS. In embodiments, the MUC16 promoter includes a nucleic acid sequence within a region from about −5400 nucleic acid residues upstream to about +200 nucleic residues downstream of a MUC16 TSS. In embodiments, the MUC16 promoter includes a nucleic acid sequence within a region from about −5200 nucleic acid residues upstream to about +200 nucleic residues downstream of a MUC16 TSS. In embodiments, the MUC16 promoter includes a nucleic acid sequence within a region from about −5000 nucleic acid residues upstream to about +200 nucleic residues downstream of a MUC16 TSS. In embodiments, the MUC16 promoter includes a nucleic acid sequence within a region from about −4800 nucleic acid residues upstream to about +200 nucleic residues downstream of a MUC16 TSS. In embodiments, the MUC16 promoter includes a nucleic acid sequence within a region from about −4600 nucleic acid residues upstream to about +200 nucleic residues downstream of a MUC16 TSS. In embodiments, the MUC16 promoter includes a nucleic acid sequence within a region from about −4400 nucleic acid residues upstream to about +200 nucleic residues downstream of a MUC16 TSS. In embodiments, the MUC16 promoter includes a nucleic acid sequence within a region from about −4200 nucleic acid residues upstream to about +200 nucleic residues downstream of a MUC16 TSS. In embodiments, the MUC16 promoter includes a nucleic acid sequence within a region from about −4000 nucleic acid residues upstream to about +200 nucleic residues downstream of a MUC16 TSS. In embodiments, the MUC16 promoter includes a nucleic acid sequence within a region from about −3800 nucleic acid residues upstream to about +200 nucleic residues downstream of a MUC16 TSS. In embodiments, the MUC16 promoter includes a nucleic acid sequence within a region from about −3600 nucleic acid residues upstream to about +200 nucleic residues downstream of a MUC16 TSS. In embodiments, the MUC16 promoter includes a nucleic acid sequence within a region from about −3400 nucleic acid residues upstream to about +200 nucleic residues downstream of a MUC16 TSS. In embodiments, the MUC16 promoter includes a nucleic acid sequence within a region from about −3200 nucleic acid residues upstream to about +200 nucleic residues downstream of a MUC16 TSS. In embodiments, the MUC16 promoter includes a nucleic acid sequence within a region from about −3000 nucleic acid residues upstream to about +200 nucleic residues downstream of a MUC16 TSS. In embodiments, the MUC16 promoter includes a nucleic acid sequence within a region from about −2800 nucleic acid residues upstream to about +200 nucleic residues downstream of a MUC16 TSS. In embodiments, the MUC16 promoter includes a nucleic acid sequence within a region from about −2600 nucleic acid residues upstream to about +200 nucleic residues downstream of a MUC16 TSS. In embodiments, the MUC16 promoter includes a nucleic acid sequence within a region from about −2400 nucleic acid residues upstream to about +200 nucleic residues downstream of a MUC16 TSS. In embodiments, the MUC16 promoter includes a nucleic acid sequence within a region from about −2200 nucleic acid residues upstream to about +200 nucleic residues downstream of a MUC16 TSS. In embodiments, the MUC16 promoter includes a nucleic acid sequence within a region from about −2000 nucleic acid residues upstream to about +200 nucleic residues downstream of a MUC16 TSS. In embodiments, the MUC16 promoter includes a nucleic acid sequence within a region from about −1800 nucleic acid residues upstream to about +200 nucleic residues downstream of a MUC16 TSS. In embodiments, the MUC16 promoter includes a nucleic acid sequence within a region from about −1600 nucleic acid residues upstream to about +200 nucleic residues downstream of a MUC16 TSS. In embodiments, the MUC16 promoter includes a nucleic acid sequence within a region from about −1400 nucleic acid residues upstream to about +200 nucleic residues downstream of a MUC16 TSS. In embodiments, the MUC16 promoter includes a nucleic acid sequence within a region from about −1200 nucleic acid residues upstream to about +200 nucleic residues downstream of a MUC16 TSS. In embodiments, the MUC16 promoter includes a nucleic acid sequence within a region from about −1000 nucleic acid residues upstream to about +200 nucleic residues downstream of a MUC16 TSS. In embodiments, the MUC16 promoter includes a nucleic acid sequence within a region from about −800 nucleic acid residues upstream to about +200 nucleic residues downstream of a MUC16 TSS. In embodiments, the MUC16 promoter includes a nucleic acid sequence within a region from about −600 nucleic acid residues upstream to about +200 nucleic residues downstream of a MUC16 TSS. In embodiments, the MUC16 promoter includes a nucleic acid sequence within a region from about −400 nucleic acid residues upstream to about +200 nucleic residues downstream of a MUC16 TSS. In embodiments, the MUC16 promoter includes a nucleic acid sequence within a region from about −200 nucleic acid residues upstream to about +200 nucleic residues downstream of a MUC16 TSS. In embodiments, the MUC16 promoter includes a nucleic acid sequence within a region from a MUC16 TSS to about +200 nucleic residues downstream of the MUC16 TSS.

In embodiments, the MUC16 promoter includes a nucleic acid sequence within a region from about −6000 nucleic acid residues upstream of the MUC16 TSS to about the MUC16 TSS. In embodiments, the MUC16 promoter includes a nucleic acid sequence within a region from about −6000 nucleic acid residues upstream to about −200 nucleic residues upstream of a MUC16 TSS. In embodiments, the MUC16 promoter includes a nucleic acid sequence within a region from about −6000 nucleic acid residues upstream to about −400 nucleic residues upstream of a MUC16 TSS. In embodiments, the MUC16 promoter includes a nucleic acid sequence within a region from about −6000 nucleic acid residues upstream to about −600 nucleic residues upstream of a MUC16 TSS. In embodiments, the MUC16 promoter includes a nucleic acid sequence within a region from about −6000 nucleic acid residues upstream to about −800 nucleic residues upstream of a MUC16 TSS. In embodiments, the MUC16 promoter includes a nucleic acid sequence within a region from about −6000 nucleic acid residues upstream to about −1000 nucleic residues upstream of a MUC16 TSS. In embodiments, the MUC16 promoter includes a nucleic acid sequence within a region from about −6000 nucleic acid residues upstream to about −1200 nucleic residues upstream of a MUC16 TSS. In embodiments, the MUC16 promoter includes a nucleic acid sequence within a region from about −6000 nucleic acid residues upstream to about −1400 nucleic residues upstream of a MUC16 TSS. In embodiments, the MUC16 promoter includes a nucleic acid sequence within a region from about −6000 nucleic acid residues upstream to about −1600 nucleic residues upstream of a MUC16 TSS. In embodiments, the MUC16 promoter includes a nucleic acid sequence within a region from about −6000 nucleic acid residues upstream to about −1800 nucleic residues upstream of a MUC16 TSS. In embodiments, the MUC16 promoter includes a nucleic acid sequence within a region from about −6000 nucleic acid residues upstream to about −2000 nucleic residues upstream of a MUC16 TSS. In embodiments, the MUC16 promoter includes a nucleic acid sequence within a region from about −6000 nucleic acid residues upstream to about −2200 nucleic residues upstream of a MUC16 TSS. In embodiments, the MUC16 promoter includes a nucleic acid sequence within a region from about −6000 nucleic acid residues upstream to about −2400 nucleic residues upstream of a MUC16 TSS. In embodiments, the MUC16 promoter includes a nucleic acid sequence within a region from about −6000 nucleic acid residues upstream to about −2600 nucleic residues upstream of a MUC16 TSS. In embodiments, the MUC16 promoter includes a nucleic acid sequence within a region from about −6000 nucleic acid residues upstream to about −2800 nucleic residues upstream of a MUC16 TSS. In embodiments, the MUC16 promoter includes a nucleic acid sequence within a region from about −6000 nucleic acid residues upstream to about −3000 nucleic residues upstream of a MUC16 TSS. In embodiments, the MUC16 promoter includes a nucleic acid sequence within a region from about −6000 nucleic acid residues upstream to about −3200 nucleic residues upstream of a MUC16 TSS. In embodiments, the MUC16 promoter includes a nucleic acid sequence within a region from about −6000 nucleic acid residues upstream to about −3400 nucleic residues upstream of a MUC16 TSS. In embodiments, the MUC16 promoter includes a nucleic acid sequence within a region from about −6000 nucleic acid residues upstream to about −3600 nucleic residues upstream of a MUC16 TSS. In embodiments, the MUC16 promoter includes a nucleic acid sequence within a region from about −6000 nucleic acid residues upstream to about −3800 nucleic residues upstream of a MUC16 TSS. In embodiments, the MUC16 promoter includes a nucleic acid sequence within a region from about −6000 nucleic acid residues upstream to about −4000 nucleic residues upstream of a MUC16 TSS. In embodiments, the MUC16 promoter includes a nucleic acid sequence within a region from about −6000 nucleic acid residues upstream to about −4200 nucleic residues upstream of a MUC16 TSS. In embodiments, the MUC16 promoter includes a nucleic acid sequence within a region from about −6000 nucleic acid residues upstream to about −4400 nucleic residues upstream of a MUC16 TSS. In embodiments, the MUC16 promoter includes a nucleic acid sequence within a region from about −6000 nucleic acid residues upstream to about −4600 nucleic residues upstream of a MUC16 TSS. In embodiments, the MUC16 promoter includes a nucleic acid sequence within a region from about −6000 nucleic acid residues upstream to about −4800 nucleic residues upstream of a MUC16 TSS. In embodiments, the MUC16 promoter includes a nucleic acid sequence within a region from about −6000 nucleic acid residues upstream to about −5000 nucleic residues upstream of a MUC16 TSS. In embodiments, the MUC16 promoter includes a nucleic acid sequence within a region from about −6000 nucleic acid residues upstream to about −5200 nucleic residues upstream of a MUC16 TSS. In embodiments, the MUC16 promoter includes a nucleic acid sequence within a region from about −6000 nucleic acid residues upstream to about −5400 nucleic residues upstream of a MUC16 TSS. In embodiments, the MUC16 promoter includes a nucleic acid sequence within a region from about −6000 nucleic acid residues upstream to about −5600 nucleic residues upstream of a MUC16 TSS. In embodiments, the MUC16 promoter includes a nucleic acid sequence within a region from about −6000 nucleic acid residues upstream to about −5800 nucleic residues upstream of a MUC16 TSS.

In embodiments, the nucleic acid sequence is from about −1200 nucleic acid residues upstream to about +100 nucleic acid residues downstream of the MUC16 TSS. In embodiments, the nucleic acid sequence is from about −1200 nucleic acid residues to about −500 nucleic residues upstream of the MUC16 TSS.

Where a “MUC16 promoter includes a nucleic acid sequence within a region,” it is understood that the nucleic acid sequence is found within a specified region relative to a MUC16 transcription start site (TSS) within a human cell. In embodiments, the specified region is from approximately position 29,720 to 34,752 of the nucleic acid sequence identified by Accession No. NG_055257.1. For example, the nucleic acid sequence may be a portion or a fragment within the region from approximately position 29,720 to 34,752 of the nucleic acid sequence identified by Accession No. NG_055257.1. In embodiments, the TSS is at a position corresponding to position 4330 of the sequence of SEQ ID NO:3. In embodiments, the TSS is at a position corresponding to position 992 of the sequence of SEQ ID NO:3. In embodiments, the human cell is a human epithelial cell. In embodiments, the human cell is a human germ cell. In embodiments, the human cell is a human stromal cell. In embodiments, the human cell is a human glandular cell. In embodiments, the human cell is a human squamous cell. In embodiments, the human cell is a CA-125 expressing cancer cell. In embodiments, the CA-125 expressing cancer cell is an ovarian cancer cell.

In embodiments, the MUC16 promoter includes a nucleic acid sequence having at least 70% sequence identity to SEQ ID NO:1. In embodiments, the MUC16 promoter includes a nucleic acid sequence having at least 71% sequence identity to SEQ ID NO:1. In embodiments, the MUC16 promoter includes a nucleic acid sequence having at least 72% sequence identity to SEQ ID NO:1. In embodiments, the MUC16 promoter includes a nucleic acid sequence having at least 73% sequence identity to SEQ ID NO:1. In embodiments, the MUC16 promoter includes a nucleic acid sequence having at least 74% sequence identity to SEQ ID NO:1. In embodiments, the MUC16 promoter includes a nucleic acid sequence having at least 75% sequence identity to SEQ ID NO:1. In embodiments, the MUC16 promoter includes a nucleic acid sequence having at least 76% sequence identity to SEQ ID NO:1. In embodiments, the MUC16 promoter includes a nucleic acid sequence having at least 77% sequence identity to SEQ ID NO:1. In embodiments, the MUC16 promoter includes a nucleic acid sequence having at least 78% sequence identity to SEQ ID NO:1. In embodiments, the MUC16 promoter includes a nucleic acid sequence having at least 79% sequence identity to SEQ ID NO:1. In embodiments, the MUC16 promoter includes a nucleic acid sequence having at least 80% sequence identity to SEQ ID NO:1. In embodiments, the MUC16 promoter includes a nucleic acid sequence having at least 81% sequence identity to SEQ ID NO:1. In embodiments, the MUC16 promoter includes a nucleic acid sequence having at least 82% sequence identity to SEQ ID NO:1. In embodiments, the MUC16 promoter includes a nucleic acid sequence having at least 83% sequence identity to SEQ ID NO:1. In embodiments, the MUC16 promoter includes a nucleic acid sequence having at least 84% sequence identity to SEQ ID NO:1. In embodiments, the MUC16 promoter includes a nucleic acid sequence having at least 85% sequence identity to SEQ ID NO:1. In embodiments, the MUC16 promoter includes a nucleic acid sequence having at least 86% sequence identity to SEQ ID NO:1. In embodiments, the MUC16 promoter includes a nucleic acid sequence having at least 87% sequence identity to SEQ ID NO:1. In embodiments, the MUC16 promoter includes a nucleic acid sequence having at least 88% sequence identity to SEQ ID NO:1. In embodiments, the MUC16 promoter includes a nucleic acid sequence having at least 89% sequence identity to SEQ ID NO:1. In embodiments, the MUC16 promoter includes a nucleic acid sequence having at least 90% sequence identity to SEQ ID NO:1. In embodiments, the MUC16 promoter includes a nucleic acid sequence having at least 91% sequence identity to SEQ ID NO:1. In embodiments, the MUC16 promoter includes a nucleic acid sequence having at least 92% sequence identity to SEQ ID NO:1. In embodiments, the MUC16 promoter includes a nucleic acid sequence having at least 93% sequence identity to SEQ ID NO:1. In embodiments, the MUC16 promoter includes a nucleic acid sequence having at least 94% sequence identity to SEQ ID NO:1. In embodiments, the MUC16 promoter includes a nucleic acid sequence having at least 95% sequence identity to SEQ ID NO:1. In embodiments, the MUC16 promoter includes a nucleic acid sequence having at least 96% sequence identity to SEQ ID NO:1. In embodiments, the MUC16 promoter includes a nucleic acid sequence having at least 97% sequence identity to SEQ ID NO:1. In embodiments, the MUC16 promoter includes a nucleic acid sequence having at least 98% sequence identity to SEQ ID NO:1. In embodiments, the MUC16 promoter includes a nucleic acid sequence having at least 99% sequence identity to SEQ ID NO: 1. In embodiments, the MUC16 promoter includes the nucleic acid sequence of SEQ ID NO: 1. In embodiments, the MUC16 promoter is the nucleic acid sequence of SEQ ID NO: 1.

In embodiments, the MUC16 promoter includes a nucleic acid sequence having about 70% sequence identity to SEQ ID NO:1. In embodiments, the MUC16 promoter includes a nucleic acid sequence having about 71% sequence identity to SEQ ID NO:1. In embodiments, the MUC16 promoter includes a nucleic acid sequence having about 72% sequence identity to SEQ ID NO:1. In embodiments, the MUC16 promoter includes a nucleic acid sequence having about 73% sequence identity to SEQ ID NO:1. In embodiments, the MUC16 promoter includes a nucleic acid sequence having about 74% sequence identity to SEQ ID NO:1. In embodiments, the MUC16 promoter includes a nucleic acid sequence having about 75% sequence identity to SEQ ID NO:1. In embodiments, the MUC16 promoter includes a nucleic acid sequence having about 76% sequence identity to SEQ ID NO:1. In embodiments, the MUC16 promoter includes a nucleic acid sequence having about 77% sequence identity to SEQ ID NO:1. In embodiments, the MUC16 promoter includes a nucleic acid sequence having about 78% sequence identity to SEQ ID NO:1. In embodiments, the MUC16 promoter includes a nucleic acid sequence having about 79% sequence identity to SEQ ID NO:1. In embodiments, the MUC16 promoter includes a nucleic acid sequence having about 80% sequence identity to SEQ ID NO:1. In embodiments, the MUC16 promoter includes a nucleic acid sequence having about 81% sequence identity to SEQ ID NO:1. In embodiments, the MUC16 promoter includes a nucleic acid sequence having about 82% sequence identity to SEQ ID NO:1. In embodiments, the MUC16 promoter includes a nucleic acid sequence having about 83% sequence identity to SEQ ID NO:1. In embodiments, the MUC16 promoter includes a nucleic acid sequence having about 84% sequence identity to SEQ ID NO:1. In embodiments, the MUC16 promoter includes a nucleic acid sequence having about 85% sequence identity to SEQ ID NO:1. In embodiments, the MUC16 promoter includes a nucleic acid sequence having about 86% sequence identity to SEQ ID NO:1. In embodiments, the MUC16 promoter includes a nucleic acid sequence having about 87% sequence identity to SEQ ID NO:1. In embodiments, the MUC16 promoter includes a nucleic acid sequence having about 88% sequence identity to SEQ ID NO:1. In embodiments, the MUC16 promoter includes a nucleic acid sequence having about 89% sequence identity to SEQ ID NO:1. In embodiments, the MUC16 promoter includes a nucleic acid sequence having about 90% sequence identity to SEQ ID NO:1. In embodiments, the MUC16 promoter includes a nucleic acid sequence having about 91% sequence identity to SEQ ID NO:1. In embodiments, the MUC16 promoter includes a nucleic acid sequence having about 92% sequence identity to SEQ ID NO:1. In embodiments, the MUC16 promoter includes a nucleic acid sequence having about 93% sequence identity to SEQ ID NO:1. In embodiments, the MUC16 promoter includes a nucleic acid sequence having about 94% sequence identity to SEQ ID NO:1. In embodiments, the MUC16 promoter includes a nucleic acid sequence having about 95% sequence identity to SEQ ID NO:1. In embodiments, the MUC16 promoter includes a nucleic acid sequence having about 96% sequence identity to SEQ ID NO:1. In embodiments, the MUC16 promoter includes a nucleic acid sequence having about 97% sequence identity to SEQ ID NO:1. In embodiments, the MUC16 promoter includes a nucleic acid sequence having about 98% sequence identity to SEQ ID NO:1. In embodiments, the MUC16 promoter includes a nucleic acid sequence having about 99% sequence identity to SEQ ID NO:1.

In embodiments, the MUC16 promoter includes a nucleic acid sequence having a sequence identity of at least 70% to SEQ ID NO:1, and the nucleic acid sequence having at least 70% sequence identity is contiguous. In embodiments, the MUC16 promoter includes a nucleic acid sequence having a sequence identity of at least 71% to SEQ ID NO:1, and the nucleic acid sequence having at least 71% sequence identity is contiguous. In embodiments, the MUC16 promoter includes a nucleic acid sequence having a sequence identity of at least 72% to SEQ ID NO:1, and the nucleic acid sequence having at least 72% sequence identity is contiguous. In embodiments, the MUC16 promoter includes a nucleic acid sequence having a sequence identity of at least 73% to SEQ ID NO:1, and the nucleic acid sequence having at least 73% sequence identity is contiguous. In embodiments, the MUC16 promoter includes a nucleic acid sequence having a sequence identity of at least 74% to SEQ ID NO:1, and the nucleic acid sequence having at least 74% sequence identity is contiguous. In embodiments, the MUC16 promoter includes a nucleic acid sequence having a sequence identity of at least 75% to SEQ ID NO:1, and the nucleic acid sequence having at least 75% sequence identity is contiguous. In embodiments, the MUC16 promoter includes a nucleic acid sequence having a sequence identity of at least 76% to SEQ ID NO:1, and the nucleic acid sequence having at least 76% sequence identity is contiguous. In embodiments, the MUC16 promoter includes a nucleic acid sequence having a sequence identity of at least 77% to SEQ ID NO:1, and the nucleic acid sequence having at least 77% sequence identity is contiguous. In embodiments, the MUC16 promoter includes a nucleic acid sequence having a sequence identity of at least 78% to SEQ ID NO:1, and the nucleic acid sequence having at least 78% sequence identity is contiguous. In embodiments, the MUC16 promoter includes a nucleic acid sequence having a sequence identity of at least 79% to SEQ ID NO:1, and the nucleic acid sequence having at least 79% sequence identity is contiguous. In embodiments, the MUC16 promoter includes a nucleic acid sequence having a sequence identity of at least 80% to SEQ ID NO:1, and the nucleic acid sequence having at least 80% sequence identity is contiguous. In embodiments, the MUC16 promoter includes a nucleic acid sequence having a sequence identity of at least 81% to SEQ ID NO:1, and the nucleic acid sequence having at least 81% sequence identity is contiguous. In embodiments, the MUC16 promoter includes a nucleic acid sequence having a sequence identity of at least 82% to SEQ ID NO:1, and the nucleic acid sequence having at least 82% sequence identity is contiguous. In embodiments, the MUC16 promoter includes a nucleic acid sequence having a sequence identity of at least 83% to SEQ ID NO:1, and the nucleic acid sequence having at least 83% sequence identity is contiguous. In embodiments, the MUC16 promoter includes a nucleic acid sequence having a sequence identity of at least 84% to SEQ ID NO:1, and the nucleic acid sequence having at least 84% sequence identity is contiguous. In embodiments, the MUC16 promoter includes a nucleic acid sequence having a sequence identity of at least 85% to SEQ ID NO:1, and the nucleic acid sequence having at least 85% sequence identity is contiguous. In embodiments, the MUC16 promoter includes a nucleic acid sequence having a sequence identity of at least 86% to SEQ ID NO:1, and the nucleic acid sequence having at least 86% sequence identity is contiguous. In embodiments, the MUC16 promoter includes a nucleic acid sequence having a sequence identity of at least 87% to SEQ ID NO:1, and the nucleic acid sequence having at least 87% sequence identity is contiguous. In embodiments, the MUC16 promoter includes a nucleic acid sequence having a sequence identity of at least 88% to SEQ ID NO:1, and the nucleic acid sequence having at least 88% sequence identity is contiguous. In embodiments, the MUC16 promoter includes a nucleic acid sequence having a sequence identity of at least 89% to SEQ ID NO:1, and the nucleic acid sequence having at least 89% sequence identity is contiguous. In embodiments, the MUC16 promoter includes a nucleic acid sequence having a sequence identity of at least 90% to SEQ ID NO:1, and the nucleic acid sequence having at least 90% sequence identity is contiguous. In embodiments, the MUC16 promoter includes a nucleic acid sequence having a sequence identity of at least 91% to SEQ ID NO:1, and the nucleic acid sequence having at least 91% sequence identity is contiguous. In embodiments, the MUC16 promoter includes a nucleic acid sequence having a sequence identity of at least 92% to SEQ ID NO:1, and the nucleic acid sequence having at least 92% sequence identity is contiguous. In embodiments, the MUC16 promoter includes a nucleic acid sequence having a sequence identity of at least 93% to SEQ ID NO:1, and the nucleic acid sequence having at least 93% sequence identity is contiguous. In embodiments, the MUC16 promoter includes a nucleic acid sequence having a sequence identity of at least 94% to SEQ ID NO:1, and the nucleic acid sequence having at least 94% sequence identity is contiguous. In embodiments, the MUC16 promoter includes a nucleic acid sequence having a sequence identity of at least 95% to SEQ ID NO:1, and the nucleic acid sequence having at least 95% sequence identity is contiguous. In embodiments, the MUC16 promoter includes a nucleic acid sequence having a sequence identity of at least 96% to SEQ ID NO:1, and the nucleic acid sequence having at least 96% sequence identity is contiguous. In embodiments, the MUC16 promoter includes a nucleic acid sequence having a sequence identity of at least 97% to SEQ ID NO:1, and the nucleic acid sequence having at least 97% sequence identity is contiguous. In embodiments, the MUC16 promoter includes a nucleic acid sequence having a sequence identity of at least 98% to SEQ ID NO:1, and the nucleic acid sequence having at least 98% sequence identity is contiguous. In embodiments, the MUC16 promoter includes a nucleic acid sequence having a sequence identity of at least 99% to SEQ ID NO:1, and the nucleic acid sequence having at least 99% sequence identity is contiguous.

In embodiments, the MUC16 promoter includes a nucleic acid sequence having at least 70% sequence identity to SEQ ID NO:2. In embodiments, the MUC16 promoter includes a nucleic acid sequence having at least 71% sequence identity to SEQ ID NO:2. In embodiments, the MUC16 promoter includes a nucleic acid sequence having at least 72% sequence identity to SEQ ID NO:2. In embodiments, the MUC16 promoter includes a nucleic acid sequence having at least 73% sequence identity to SEQ ID NO:2. In embodiments, the MUC16 promoter includes a nucleic acid sequence having at least 74% sequence identity to SEQ ID NO:2. In embodiments, the MUC16 promoter includes a nucleic acid sequence having at least 75% sequence identity to SEQ ID NO:2. In embodiments, the MUC16 promoter includes a nucleic acid sequence having at least 76% sequence identity to SEQ ID NO:2. In embodiments, the MUC16 promoter includes a nucleic acid sequence having at least 77% sequence identity to SEQ ID NO:2. In embodiments, the MUC16 promoter includes a nucleic acid sequence having at least 78% sequence identity to SEQ ID NO:2. In embodiments, the MUC16 promoter includes a nucleic acid sequence having at least 79% sequence identity to SEQ ID NO:2. In embodiments, the MUC16 promoter includes a nucleic acid sequence having at least 80% sequence identity to SEQ ID NO:2. In embodiments, the MUC16 promoter includes a nucleic acid sequence having at least 81% sequence identity to SEQ ID NO:2. In embodiments, the MUC16 promoter includes a nucleic acid sequence having at least 82% sequence identity to SEQ ID NO:2. In embodiments, the MUC16 promoter includes a nucleic acid sequence having at least 83% sequence identity to SEQ ID NO:2. In embodiments, the MUC16 promoter includes a nucleic acid sequence having at least 84% sequence identity to SEQ ID NO:2. In embodiments, the MUC16 promoter includes a nucleic acid sequence having at least 85% sequence identity to SEQ ID NO:2. In embodiments, the MUC16 promoter includes a nucleic acid sequence having at least 86% sequence identity to SEQ ID NO:2. In embodiments, the MUC16 promoter includes a nucleic acid sequence having at least 87% sequence identity to SEQ ID NO:2. In embodiments, the MUC16 promoter includes a nucleic acid sequence having at least 88% sequence identity to SEQ ID NO:2. In embodiments, the MUC16 promoter includes a nucleic acid sequence having at least 89% sequence identity to SEQ ID NO:2. In embodiments, the MUC16 promoter includes a nucleic acid sequence having at least 90% sequence identity to SEQ ID NO:2. In embodiments, the MUC16 promoter includes a nucleic acid sequence having at least 91% sequence identity to SEQ ID NO:2. In embodiments, the MUC16 promoter includes a nucleic acid sequence having at least 92% sequence identity to SEQ ID NO:2. In embodiments, the MUC16 promoter includes a nucleic acid sequence having at least 93% sequence identity to SEQ ID NO:2. In embodiments, the MUC16 promoter includes a nucleic acid sequence having at least 94% sequence identity to SEQ ID NO:2. In embodiments, the MUC16 promoter includes a nucleic acid sequence having at least 95% sequence identity to SEQ ID NO:2. In embodiments, the MUC16 promoter includes a nucleic acid sequence having at least 96% sequence identity to SEQ ID NO:2. In embodiments, the MUC16 promoter includes a nucleic acid sequence having at least 97% sequence identity to SEQ ID NO:2. In embodiments, the MUC16 promoter includes a nucleic acid sequence having at least 98% sequence identity to SEQ ID NO:2. In embodiments, the MUC16 promoter includes a nucleic acid sequence having at least 99% sequence identity to SEQ ID NO:2. In embodiments, the MUC16 promoter includes the nucleic acid sequence of SEQ ID NO:2. In embodiments, the MUC16 promoter is the nucleic acid sequence of SEQ ID NO:2.

In embodiments, the MUC16 promoter includes a nucleic acid sequence having about 70% sequence identity to SEQ ID NO:2. In embodiments, the MUC16 promoter includes a nucleic acid sequence having about 71% sequence identity to SEQ ID NO:2. In embodiments, the MUC16 promoter includes a nucleic acid sequence having about 72% sequence identity to SEQ ID NO:2. In embodiments, the MUC16 promoter includes a nucleic acid sequence having about 73% sequence identity to SEQ ID NO:2. In embodiments, the MUC16 promoter includes a nucleic acid sequence having about 74% sequence identity to SEQ ID NO:2. In embodiments, the MUC16 promoter includes a nucleic acid sequence having about 75% sequence identity to SEQ ID NO:2. In embodiments, the MUC16 promoter includes a nucleic acid sequence having about 76% sequence identity to SEQ ID NO:2. In embodiments, the MUC16 promoter includes a nucleic acid sequence having about 77% sequence identity to SEQ ID NO:2. In embodiments, the MUC16 promoter includes a nucleic acid sequence having about 78% sequence identity to SEQ ID NO:2. In embodiments, the MUC16 promoter includes a nucleic acid sequence having about 79% sequence identity to SEQ ID NO:2. In embodiments, the MUC16 promoter includes a nucleic acid sequence having about 80% sequence identity to SEQ ID NO:2. In embodiments, the MUC16 promoter includes a nucleic acid sequence having about 81% sequence identity to SEQ ID NO:2. In embodiments, the MUC16 promoter includes a nucleic acid sequence having about 82% sequence identity to SEQ ID NO:2. In embodiments, the MUC16 promoter includes a nucleic acid sequence having about 83% sequence identity to SEQ ID NO:2. In embodiments, the MUC16 promoter includes a nucleic acid sequence having about 84% sequence identity to SEQ ID NO:2. In embodiments, the MUC16 promoter includes a nucleic acid sequence having about 85% sequence identity to SEQ ID NO:2. In embodiments, the MUC16 promoter includes a nucleic acid sequence having about 86% sequence identity to SEQ ID NO:2. In embodiments, the MUC16 promoter includes a nucleic acid sequence having about 87% sequence identity to SEQ ID NO:2. In embodiments, the MUC16 promoter includes a nucleic acid sequence having about 88% sequence identity to SEQ ID NO:2. In embodiments, the MUC16 promoter includes a nucleic acid sequence having about 89% sequence identity to SEQ ID NO:2. In embodiments, the MUC16 promoter includes a nucleic acid sequence having about 90% sequence identity to SEQ ID NO:2. In embodiments, the MUC16 promoter includes a nucleic acid sequence having about 91% sequence identity to SEQ ID NO:2. In embodiments, the MUC16 promoter includes a nucleic acid sequence having about 92% sequence identity to SEQ ID NO:2. In embodiments, the MUC16 promoter includes a nucleic acid sequence having about 93% sequence identity to SEQ ID NO:2. In embodiments, the MUC16 promoter includes a nucleic acid sequence having about 94% sequence identity to SEQ ID NO:2. In embodiments, the MUC16 promoter includes a nucleic acid sequence having about 95% sequence identity to SEQ ID NO:2. In embodiments, the MUC16 promoter includes a nucleic acid sequence having about 96% sequence identity to SEQ ID NO:2. In embodiments, the MUC16 promoter includes a nucleic acid sequence having about 97% sequence identity to SEQ ID NO:2. In embodiments, the MUC16 promoter includes a nucleic acid sequence having about 98% sequence identity to SEQ ID NO:2. In embodiments, the MUC16 promoter includes a nucleic acid sequence having about 99% sequence identity to SEQ ID NO:2.

In embodiments, the MUC16 promoter includes a nucleic acid sequence having a sequence identity of at least 70% to SEQ ID NO:2, and the nucleic acid sequence having at least 70% sequence identity is contiguous. In embodiments, the MUC16 promoter includes a nucleic acid sequence having a sequence identity of at least 71% to SEQ ID NO:2, and the nucleic acid sequence having at least 71% sequence identity is contiguous. In embodiments, the MUC16 promoter includes a nucleic acid sequence having a sequence identity of at least 72% to SEQ ID NO:2, and the nucleic acid sequence having at least 72% sequence identity is contiguous. In embodiments, the MUC16 promoter includes a nucleic acid sequence having a sequence identity of at least 73% to SEQ ID NO:2, and the nucleic acid sequence having at least 73% sequence identity is contiguous. In embodiments, the MUC16 promoter includes a nucleic acid sequence having a sequence identity of at least 74% to SEQ ID NO:2, and the nucleic acid sequence having at least 74% sequence identity is contiguous. In embodiments, the MUC16 promoter includes a nucleic acid sequence having a sequence identity of at least 75% to SEQ ID NO:2, and the nucleic acid sequence having at least 75% sequence identity is contiguous. In embodiments, the MUC16 promoter includes a nucleic acid sequence having a sequence identity of at least 76% to SEQ ID NO:2, and the nucleic acid sequence having at least 76% sequence identity is contiguous. In embodiments, the MUC16 promoter includes a nucleic acid sequence having a sequence identity of at least 77% to SEQ ID NO:2, and the nucleic acid sequence having at least 77% sequence identity is contiguous. In embodiments, the MUC16 promoter includes a nucleic acid sequence having a sequence identity of at least 78% to SEQ ID NO:2, and the nucleic acid sequence having at least 78% sequence identity is contiguous. In embodiments, the MUC16 promoter includes a nucleic acid sequence having a sequence identity of at least 79% to SEQ ID NO:2, and the nucleic acid sequence having at least 79% sequence identity is contiguous. In embodiments, the MUC16 promoter includes a nucleic acid sequence having a sequence identity of at least 80% to SEQ ID NO:2, and the nucleic acid sequence having at least 80% sequence identity is contiguous. In embodiments, the MUC16 promoter includes a nucleic acid sequence having a sequence identity of at least 81% to SEQ ID NO:2, and the nucleic acid sequence having at least 81% sequence identity is contiguous. In embodiments, the MUC16 promoter includes a nucleic acid sequence having a sequence identity of at least 82% to SEQ ID NO:2, and the nucleic acid sequence having at least 82% sequence identity is contiguous. In embodiments, the MUC16 promoter includes a nucleic acid sequence having a sequence identity of at least 83% to SEQ ID NO:2, and the nucleic acid sequence having at least 83% sequence identity is contiguous. In embodiments, the MUC16 promoter includes a nucleic acid sequence having a sequence identity of at least 84% to SEQ ID NO:2, and the nucleic acid sequence having at least 84% sequence identity is contiguous. In embodiments, the MUC16 promoter includes a nucleic acid sequence having a sequence identity of at least 85% to SEQ ID NO:2, and the nucleic acid sequence having at least 85% sequence identity is contiguous. In embodiments, the MUC16 promoter includes a nucleic acid sequence having a sequence identity of at least 86% to SEQ ID NO:2, and the nucleic acid sequence having at least 86% sequence identity is contiguous. In embodiments, the MUC16 promoter includes a nucleic acid sequence having a sequence identity of at least 87% to SEQ ID NO:2, and the nucleic acid sequence having at least 87% sequence identity is contiguous. In embodiments, the MUC16 promoter includes a nucleic acid sequence having a sequence identity of at least 88% to SEQ ID NO:2, and the nucleic acid sequence having at least 88% sequence identity is contiguous. In embodiments, the MUC16 promoter includes a nucleic acid sequence having a sequence identity of at least 89% to SEQ ID NO:2, and the nucleic acid sequence having at least 89% sequence identity is contiguous. In embodiments, the MUC16 promoter includes a nucleic acid sequence having a sequence identity of at least 90% to SEQ ID NO:2, and the nucleic acid sequence having at least 90% sequence identity is contiguous. In embodiments, the MUC16 promoter includes a nucleic acid sequence having a sequence identity of at least 91% to SEQ ID NO:2, and the nucleic acid sequence having at least 91% sequence identity is contiguous. In embodiments, the MUC16 promoter includes a nucleic acid sequence having a sequence identity of at least 92% to SEQ ID NO:2, and the nucleic acid sequence having at least 92% sequence identity is contiguous. In embodiments, the MUC16 promoter includes a nucleic acid sequence having a sequence identity of at least 93% to SEQ ID NO:2, and the nucleic acid sequence having at least 93% sequence identity is contiguous. In embodiments, the MUC16 promoter includes a nucleic acid sequence having a sequence identity of at least 94% to SEQ ID NO:2, and the nucleic acid sequence having at least 94% sequence identity is contiguous. In embodiments, the MUC16 promoter includes a nucleic acid sequence having a sequence identity of at least 95% to SEQ ID NO:2, and the nucleic acid sequence having at least 95% sequence identity is contiguous. In embodiments, the MUC16 promoter includes a nucleic acid sequence having a sequence identity of at least 96% to SEQ ID NO:2, and the nucleic acid sequence having at least 96% sequence identity is contiguous. In embodiments, the MUC16 promoter includes a nucleic acid sequence having a sequence identity of at least 97% to SEQ ID NO:2, and the nucleic acid sequence having at least 97% sequence identity is contiguous. In embodiments, the MUC16 promoter includes a nucleic acid sequence having a sequence identity of at least 98% to SEQ ID NO:2, and the nucleic acid sequence having at least 98% sequence identity is contiguous. In embodiments, the MUC16 promoter includes a nucleic acid sequence having a sequence identity of at least 99% to SEQ ID NO:2, and the nucleic acid sequence having at least 99% sequence identity is contiguous.

In embodiments, the MUC16 promoter includes a nucleic acid sequence having at least 50% sequence identity to SEQ ID NO:3. In embodiments, the MUC16 promoter includes a nucleic acid sequence having at least 51% sequence identity to SEQ ID NO:3. In embodiments, the MUC16 promoter includes a nucleic acid sequence having at least 52% sequence identity to SEQ ID NO:3. In embodiments, the MUC16 promoter includes a nucleic acid sequence having at least 53% sequence identity to SEQ ID NO:3. In embodiments, the MUC16 promoter includes a nucleic acid sequence having at least 54% sequence identity to SEQ ID NO:3. In embodiments, the MUC16 promoter includes a nucleic acid sequence having at least 55% sequence identity to SEQ ID NO:3. In embodiments, the MUC16 promoter includes a nucleic acid sequence having at least 56% sequence identity to SEQ ID NO:3. In embodiments, the MUC16 promoter includes a nucleic acid sequence having at least 57% sequence identity to SEQ ID NO:3. In embodiments, the MUC16 promoter includes a nucleic acid sequence having at least 58% sequence identity to SEQ ID NO:3. In embodiments, the MUC16 promoter includes a nucleic acid sequence having at least 59% sequence identity to SEQ ID NO:3. In embodiments, the MUC16 promoter includes a nucleic acid sequence having at least 60% sequence identity to SEQ ID NO:3. In embodiments, the MUC16 promoter includes a nucleic acid sequence having at least 61% sequence identity to SEQ ID NO:3. In embodiments, the MUC16 promoter includes a nucleic acid sequence having at least 62% sequence identity to SEQ ID NO:3. In embodiments, the MUC16 promoter includes a nucleic acid sequence having at least 63% sequence identity to SEQ ID NO:3. In embodiments, the MUC16 promoter includes a nucleic acid sequence having at least 64% sequence identity to SEQ ID NO:3. In embodiments, the MUC16 promoter includes a nucleic acid sequence having at least 65% sequence identity to SEQ ID NO:3. In embodiments, the MUC16 promoter includes a nucleic acid sequence having at least 66% sequence identity to SEQ ID NO:3. In embodiments, the MUC16 promoter includes a nucleic acid sequence having at least 67% sequence identity to SEQ ID NO:3. In embodiments, the MUC16 promoter includes a nucleic acid sequence having at least 68% sequence identity to SEQ ID NO:3. In embodiments, the MUC16 promoter includes a nucleic acid sequence having at least 69% sequence identity to SEQ ID NO:3. In embodiments, the MUC16 promoter includes a nucleic acid sequence having at least 70% sequence identity to SEQ ID NO:3. In embodiments, the MUC16 promoter includes a nucleic acid sequence having at least 70% sequence identity to SEQ ID NO:3. In embodiments, the MUC16 promoter includes a nucleic acid sequence having at least 71% sequence identity to SEQ ID NO:3. In embodiments, the MUC16 promoter includes a nucleic acid sequence having at least 72% sequence identity to SEQ ID NO:3. In embodiments, the MUC16 promoter includes a nucleic acid sequence having at least 73% sequence identity to SEQ ID NO:3. In embodiments, the MUC16 promoter includes a nucleic acid sequence having at least 74% sequence identity to SEQ ID NO:3. In embodiments, the MUC16 promoter includes a nucleic acid sequence having at least 75% sequence identity to SEQ ID NO:3. In embodiments, the MUC16 promoter includes a nucleic acid sequence having at least 76% sequence identity to SEQ ID NO:3. In embodiments, the MUC16 promoter includes a nucleic acid sequence having at least 77% sequence identity to SEQ ID NO:3. In embodiments, the MUC16 promoter includes a nucleic acid sequence having at least 78% sequence identity to SEQ ID NO:3. In embodiments, the MUC16 promoter includes a nucleic acid sequence having at least 79% sequence identity to SEQ ID NO:3. In embodiments, the MUC16 promoter includes a nucleic acid sequence having at least 80% sequence identity to SEQ ID NO:3. In embodiments, the MUC16 promoter includes a nucleic acid sequence having at least 81% sequence identity to SEQ ID NO:3. In embodiments, the MUC16 promoter includes a nucleic acid sequence having at least 82% sequence identity to SEQ ID NO:3. In embodiments, the MUC16 promoter includes a nucleic acid sequence having at least 83% sequence identity to SEQ ID NO:3. In embodiments, the MUC16 promoter includes a nucleic acid sequence having at least 84% sequence identity to SEQ ID NO:3. In embodiments, the MUC16 promoter includes a nucleic acid sequence having at least 85% sequence identity to SEQ ID NO:3. In embodiments, the MUC16 promoter includes a nucleic acid sequence having at least 86% sequence identity to SEQ ID NO:3. In embodiments, the MUC16 promoter includes a nucleic acid sequence having at least 87% sequence identity to SEQ ID NO:3. In embodiments, the MUC16 promoter includes a nucleic acid sequence having at least 88% sequence identity to SEQ ID NO:3. In embodiments, the MUC16 promoter includes a nucleic acid sequence having at least 89% sequence identity to SEQ ID NO:3. In embodiments, the MUC16 promoter includes a nucleic acid sequence having at least 90% sequence identity to SEQ ID NO:3. In embodiments, the MUC16 promoter includes a nucleic acid sequence having at least 91% sequence identity to SEQ ID NO:3. In embodiments, the MUC16 promoter includes a nucleic acid sequence having at least 92% sequence identity to SEQ ID NO:3. In embodiments, the MUC16 promoter includes a nucleic acid sequence having at least 93% sequence identity to SEQ ID NO:3. In embodiments, the MUC16 promoter includes a nucleic acid sequence having at least 94% sequence identity to SEQ ID NO:3. In embodiments, the MUC16 promoter includes a nucleic acid sequence having at least 95% sequence identity to SEQ ID NO:3. In embodiments, the MUC16 promoter includes a nucleic acid sequence having at least 96% sequence identity to SEQ ID NO:3. In embodiments, the MUC16 promoter includes a nucleic acid sequence having at least 97% sequence identity to SEQ ID NO:3. In embodiments, the MUC16 promoter includes a nucleic acid sequence having at least 98% sequence identity to SEQ ID NO:3. In embodiments, the MUC16 promoter includes a nucleic acid sequence having at least 99% sequence identity to SEQ ID NO:3. In embodiments, the MUC16 promoter includes the nucleic acid sequence of SEQ ID NO:3. In embodiments, the MUC16 promoter is the nucleic acid sequence of SEQ ID NO:3.

In embodiments, the MUC16 promoter includes a nucleic acid sequence having about 50% sequence identity to SEQ ID NO:3. In embodiments, the MUC16 promoter includes a nucleic acid sequence having about 51% sequence identity to SEQ ID NO:3. In embodiments, the MUC16 promoter includes a nucleic acid sequence having about 52% sequence identity to SEQ ID NO:3. In embodiments, the MUC16 promoter includes a nucleic acid sequence having about 53% sequence identity to SEQ ID NO:3. In embodiments, the MUC16 promoter includes a nucleic acid sequence having about 54% sequence identity to SEQ ID NO:3. In embodiments, the MUC16 promoter includes a nucleic acid sequence having about 55% sequence identity to SEQ ID NO:3. In embodiments, the MUC16 promoter includes a nucleic acid sequence having about 56% sequence identity to SEQ ID NO:3. In embodiments, the MUC16 promoter includes a nucleic acid sequence having about 57% sequence identity to SEQ ID NO:3. In embodiments, the MUC16 promoter includes a nucleic acid sequence having about 58% sequence identity to SEQ ID NO:3. In embodiments, the MUC16 promoter includes a nucleic acid sequence having about 59% sequence identity to SEQ ID NO:3. In embodiments, the MUC16 promoter includes a nucleic acid sequence having about 60% sequence identity to SEQ ID NO:3. In embodiments, the MUC16 promoter includes a nucleic acid sequence having about 61% sequence identity to SEQ ID NO:3. In embodiments, the MUC16 promoter includes a nucleic acid sequence having about 62% sequence identity to SEQ ID NO:3. In embodiments, the MUC16 promoter includes a nucleic acid sequence having about 63% sequence identity to SEQ ID NO:3. In embodiments, the MUC16 promoter includes a nucleic acid sequence having about 64% sequence identity to SEQ ID NO:3. In embodiments, the MUC16 promoter includes a nucleic acid sequence having about 65% sequence identity to SEQ ID NO:3. In embodiments, the MUC16 promoter includes a nucleic acid sequence having about 66% sequence identity to SEQ ID NO:3. In embodiments, the MUC16 promoter includes a nucleic acid sequence having about 67% sequence identity to SEQ ID NO:3. In embodiments, the MUC16 promoter includes a nucleic acid sequence having about 68% sequence identity to SEQ ID NO:3. In embodiments, the MUC16 promoter includes a nucleic acid sequence having about 69% sequence identity to SEQ ID NO:3. In embodiments, the MUC16 promoter includes a nucleic acid sequence having about 70% sequence identity to SEQ ID NO:3. In embodiments, the MUC16 promoter includes a nucleic acid sequence having about 71% sequence identity to SEQ ID NO:3. In embodiments, the MUC16 promoter includes a nucleic acid sequence having about 72% sequence identity to SEQ ID NO:3. In embodiments, the MUC16 promoter includes a nucleic acid sequence having about 73% sequence identity to SEQ ID NO:3. In embodiments, the MUC16 promoter includes a nucleic acid sequence having about 74% sequence identity to SEQ ID NO:3. In embodiments, the MUC16 promoter includes a nucleic acid sequence having about 75% sequence identity to SEQ ID NO:3. In embodiments, the MUC16 promoter includes a nucleic acid sequence having about 76% sequence identity to SEQ ID NO:3. In embodiments, the MUC16 promoter includes a nucleic acid sequence having about 77% sequence identity to SEQ ID NO:3. In embodiments, the MUC16 promoter includes a nucleic acid sequence having about 78% sequence identity to SEQ ID NO:3. In embodiments, the MUC16 promoter includes a nucleic acid sequence having about 79% sequence identity to SEQ ID NO:3. In embodiments, the MUC16 promoter includes a nucleic acid sequence having about 80% sequence identity to SEQ ID NO:3. In embodiments, the MUC16 promoter includes a nucleic acid sequence having about 81% sequence identity to SEQ ID NO:3. In embodiments, the MUC16 promoter includes a nucleic acid sequence having about 82% sequence identity to SEQ ID NO:3. In embodiments, the MUC16 promoter includes a nucleic acid sequence having about 83% sequence identity to SEQ ID NO:3. In embodiments, the MUC16 promoter includes a nucleic acid sequence having about 84% sequence identity to SEQ ID NO:3. In embodiments, the MUC16 promoter includes a nucleic acid sequence having about 85% sequence identity to SEQ ID NO:3. In embodiments, the MUC16 promoter includes a nucleic acid sequence having about 86% sequence identity to SEQ ID NO:3. In embodiments, the MUC16 promoter includes a nucleic acid sequence having about 87% sequence identity to SEQ ID NO:3. In embodiments, the MUC16 promoter includes a nucleic acid sequence having about 88% sequence identity to SEQ ID NO:3. In embodiments, the MUC16 promoter includes a nucleic acid sequence having about 89% sequence identity to SEQ ID NO:3. In embodiments, the MUC16 promoter includes a nucleic acid sequence having about 90% sequence identity to SEQ ID NO:3. In embodiments, the MUC16 promoter includes a nucleic acid sequence having about 91% sequence identity to SEQ ID NO:3. In embodiments, the MUC16 promoter includes a nucleic acid sequence having about 92% sequence identity to SEQ ID NO:3. In embodiments, the MUC16 promoter includes a nucleic acid sequence having about 93% sequence identity to SEQ ID NO:3. In embodiments, the MUC16 promoter includes a nucleic acid sequence having about 94% sequence identity to SEQ ID NO:3. In embodiments, the MUC16 promoter includes a nucleic acid sequence having about 95% sequence identity to SEQ ID NO:3. In embodiments, the MUC16 promoter includes a nucleic acid sequence having about 96% sequence identity to SEQ ID NO:3. In embodiments, the MUC16 promoter includes a nucleic acid sequence having about 97% sequence identity to SEQ ID NO:3. In embodiments, the MUC16 promoter includes a nucleic acid sequence having about 98% sequence identity to SEQ ID NO:3. In embodiments, the MUC16 promoter includes a nucleic acid sequence having about 99% sequence identity to SEQ ID NO:3.

In embodiments, the MUC16 promoter includes a nucleic acid sequence having a sequence identity of at least 50% to SEQ ID NO:3, and the nucleic acid sequence having at least 50% sequence identity is contiguous. In embodiments, the MUC16 promoter includes a nucleic acid sequence having a sequence identity of at least 51% to SEQ ID NO:3, and the nucleic acid sequence having at least 51% sequence identity is contiguous. In embodiments, the MUC16 promoter includes a nucleic acid sequence having a sequence identity of at least 52% to SEQ ID NO:3, and the nucleic acid sequence having at least 52% sequence identity is contiguous. In embodiments, the MUC16 promoter includes a nucleic acid sequence having a sequence identity of at least 53% to SEQ ID NO:3, and the nucleic acid sequence having at least 53% sequence identity is contiguous. In embodiments, the MUC16 promoter includes a nucleic acid sequence having a sequence identity of at least 54% to SEQ ID NO:3, and the nucleic acid sequence having at least 54% sequence identity is contiguous. In embodiments, the MUC16 promoter includes a nucleic acid sequence having a sequence identity of at least 55% to SEQ ID NO:3, and the nucleic acid sequence having at least 55% sequence identity is contiguous. In embodiments, the MUC16 promoter includes a nucleic acid sequence having a sequence identity of at least 56% to SEQ ID NO:3, and the nucleic acid sequence having at least 56% sequence identity is contiguous. In embodiments, the MUC16 promoter includes a nucleic acid sequence having a sequence identity of at least 57% to SEQ ID NO:3, and the nucleic acid sequence having at least 57% sequence identity is contiguous. In embodiments, the MUC16 promoter includes a nucleic acid sequence having a sequence identity of at least 58% to SEQ ID NO:3, and the nucleic acid sequence having at least 58% sequence identity is contiguous. In embodiments, the MUC16 promoter includes a nucleic acid sequence having a sequence identity of at least 59% to SEQ ID NO:3, and the nucleic acid sequence having at least 59% sequence identity is contiguous. In embodiments, the MUC16 promoter includes a nucleic acid sequence having a sequence identity of at least 60% to SEQ ID NO:3, and the nucleic acid sequence having at least 60% sequence identity is contiguous. In embodiments, the MUC16 promoter includes a nucleic acid sequence having a sequence identity of at least 61% to SEQ ID NO:3, and the nucleic acid sequence having at least 61% sequence identity is contiguous. In embodiments, the MUC16 promoter includes a nucleic acid sequence having a sequence identity of at least 62% to SEQ ID NO:3, and the nucleic acid sequence having at least 62% sequence identity is contiguous. In embodiments, the MUC16 promoter includes a nucleic acid sequence having a sequence identity of at least 63% to SEQ ID NO:3, and the nucleic acid sequence having at least 63% sequence identity is contiguous. In embodiments, the MUC16 promoter includes a nucleic acid sequence having a sequence identity of at least 64% to SEQ ID NO:3, and the nucleic acid sequence having at least 64% sequence identity is contiguous. In embodiments, the MUC16 promoter includes a nucleic acid sequence having a sequence identity of at least 65% to SEQ ID NO:3, and the nucleic acid sequence having at least 65% sequence identity is contiguous. In embodiments, the MUC16 promoter includes a nucleic acid sequence having a sequence identity of at least 66% to SEQ ID NO:3, and the nucleic acid sequence having at least 66% sequence identity is contiguous. In embodiments, the MUC16 promoter includes a nucleic acid sequence having a sequence identity of at least 67% to SEQ ID NO:3, and the nucleic acid sequence having at least 67% sequence identity is contiguous. In embodiments, the MUC16 promoter includes a nucleic acid sequence having a sequence identity of at least 68% to SEQ ID NO:3, and the nucleic acid sequence having at least 68% sequence identity is contiguous. In embodiments, the MUC16 promoter includes a nucleic acid sequence having a sequence identity of at least 69% to SEQ ID NO:3, and the nucleic acid sequence having at least 69% sequence identity is contiguous. In embodiments, the MUC16 promoter includes a nucleic acid sequence having a sequence identity of at least 70% to SEQ ID NO:3, and the nucleic acid sequence having at least 70% sequence identity is contiguous. In embodiments, the MUC16 promoter includes a nucleic acid sequence having a sequence identity of at least 71% to SEQ ID NO:3, and the nucleic acid sequence having at least 71% sequence identity is contiguous. In embodiments, the MUC16 promoter includes a nucleic acid sequence having a sequence identity of at least 72% to SEQ ID NO:3, and the nucleic acid sequence having at least 72% sequence identity is contiguous. In embodiments, the MUC16 promoter includes a nucleic acid sequence having a sequence identity of at least 73% to SEQ ID NO:3, and the nucleic acid sequence having at least 73% sequence identity is contiguous. In embodiments, the MUC16 promoter includes a nucleic acid sequence having a sequence identity of at least 74% to SEQ ID NO:3, and the nucleic acid sequence having at least 74% sequence identity is contiguous. In embodiments, the MUC16 promoter includes a nucleic acid sequence having a sequence identity of at least 75% to SEQ ID NO:3, and the nucleic acid sequence having at least 75% sequence identity is contiguous. In embodiments, the MUC16 promoter includes a nucleic acid sequence having a sequence identity of at least 76% to SEQ ID NO:3, and the nucleic acid sequence having at least 76% sequence identity is contiguous. In embodiments, the MUC16 promoter includes a nucleic acid sequence having a sequence identity of at least 77% to SEQ ID NO:3, and the nucleic acid sequence having at least 77% sequence identity is contiguous. In embodiments, the MUC16 promoter includes a nucleic acid sequence having a sequence identity of at least 78% to SEQ ID NO:3, and the nucleic acid sequence having at least 78% sequence identity is contiguous. In embodiments, the MUC16 promoter includes a nucleic acid sequence having a sequence identity of at least 79% to SEQ ID NO:3, and the nucleic acid sequence having at least 79% sequence identity is contiguous. In embodiments, the MUC16 promoter includes a nucleic acid sequence having a sequence identity of at least 80% to SEQ ID NO:3, and the nucleic acid sequence having at least 80% sequence identity is contiguous. In embodiments, the MUC16 promoter includes a nucleic acid sequence having a sequence identity of at least 81% to SEQ ID NO:3, and the nucleic acid sequence having at least 81% sequence identity is contiguous. In embodiments, the MUC16 promoter includes a nucleic acid sequence having a sequence identity of at least 82% to SEQ ID NO:3, and the nucleic acid sequence having at least 82% sequence identity is contiguous. In embodiments, the MUC16 promoter includes a nucleic acid sequence having a sequence identity of at least 83% to SEQ ID NO:3, and the nucleic acid sequence having at least 83% sequence identity is contiguous. In embodiments, the MUC16 promoter includes a nucleic acid sequence having a sequence identity of at least 84% to SEQ ID NO:3, and the nucleic acid sequence having at least 84% sequence identity is contiguous. In embodiments, the MUC16 promoter includes a nucleic acid sequence having a sequence identity of at least 85% to SEQ ID NO:3, and the nucleic acid sequence having at least 85% sequence identity is contiguous. In embodiments, the MUC16 promoter includes a nucleic acid sequence having a sequence identity of at least 86% to SEQ ID NO:3, and the nucleic acid sequence having at least 86% sequence identity is contiguous. In embodiments, the MUC16 promoter includes a nucleic acid sequence having a sequence identity of at least 87% to SEQ ID NO:3, and the nucleic acid sequence having at least 87% sequence identity is contiguous. In embodiments, the MUC16 promoter includes a nucleic acid sequence having a sequence identity of at least 88% to SEQ ID NO:3, and the nucleic acid sequence having at least 88% sequence identity is contiguous. In embodiments, the MUC16 promoter includes a nucleic acid sequence having a sequence identity of at least 89% to SEQ ID NO:3, and the nucleic acid sequence having at least 89% sequence identity is contiguous. In embodiments, the MUC16 promoter includes a nucleic acid sequence having a sequence identity of at least 90% to SEQ ID NO:3, and the nucleic acid sequence having at least 90% sequence identity is contiguous. In embodiments, the MUC16 promoter includes a nucleic acid sequence having a sequence identity of at least 91% to SEQ ID NO:3, and the nucleic acid sequence having at least 91% sequence identity is contiguous. In embodiments, the MUC16 promoter includes a nucleic acid sequence having a sequence identity of at least 92% to SEQ ID NO:3, and the nucleic acid sequence having at least 92% sequence identity is contiguous. In embodiments, the MUC16 promoter includes a nucleic acid sequence having a sequence identity of at least 93% to SEQ ID NO:3, and the nucleic acid sequence having at least 93% sequence identity is contiguous. In embodiments, the MUC16 promoter includes a nucleic acid sequence having a sequence identity of at least 94% to SEQ ID NO:3, and the nucleic acid sequence having at least 94% sequence identity is contiguous. In embodiments, the MUC16 promoter includes a nucleic acid sequence having a sequence identity of at least 95% to SEQ ID NO:3, and the nucleic acid sequence having at least 95% sequence identity is contiguous. In embodiments, the MUC16 promoter includes a nucleic acid sequence having a sequence identity of at least 96% to SEQ ID NO:3, and the nucleic acid sequence having at least 96% sequence identity is contiguous. In embodiments, the MUC16 promoter includes a nucleic acid sequence having a sequence identity of at least 97% to SEQ ID NO:3, and the nucleic acid sequence having at least 97% sequence identity is contiguous. In embodiments, the MUC16 promoter includes a nucleic acid sequence having a sequence identity of at least 98% to SEQ ID NO:3, and the nucleic acid sequence having at least 98% sequence identity is contiguous. In embodiments, the MUC16 promoter includes a nucleic acid sequence having a sequence identity of at least 99% to SEQ ID NO:3, and the nucleic acid sequence having at least 99% sequence identity is contiguous.

In embodiments, the virus further includes a nucleic acid sequence encoding one or more anti-cancer proteins. As used herein, the terms “anti-cancer protein” or “anti-cancer proteins” refer to proteins having antineoplastic properties and/or the ability to inhibit the growth or proliferation of a cancer cell and/or provide for selective expression of the virus provided herein including embodiments thereof in a cancer cell relative to a healthy cell. Anti-cancer proteins may inhibit the progression or slow the progression of cancer temporarily or permanently. For example, an anti-cancer protein may directly or indirectly inhibit or downregulate proliferation of cancer cells. For example, an anti-cancer protein may directly or indirectly upregulate or increase production of compounds that inhibit or downregulate proliferation of cancer cells. In other examples, an anti-cancer protein kills cancer cells or increases production of compounds that kill cancer cells. In examples, an anti-cancer protein activates signaling pathways that inhibit proliferation of cancer cells or kill cancer cells. In embodiments, an anti-cancer protein is a polypeptide (e.g. antibody or fragment thereof) capable of de-repressing anti-tumor immune responses, for example an immune checkpoint inhibitor (e.g., anti-PD-L1 antibodies or fragments thereof).

In embodiments, the nucleic acid sequence encoding one or more anti-cancer proteins may be inserted into a non-essential viral gene. Genes that are not required for expression and replication of the virus are referred to herein as “non-essential genes”. The nucleic acid sequence encoding one or more anti-cancer proteins may be incorporated into the virus genome through insertion into genes or may be operably linked to genes. Upon insertion of the nucleic acid sequence into a virus gene, the gene (e.g., non-essential gene) or portions thereof may be deleted. In embodiments, the nucleic acid sequence encoding one or more anti-cancer proteins form part of a non-essential gene of the virus provided herein. In embodiments, the nucleic acid sequence encoding one or more anti-cancer proteins are inserted into a non-essential gene of the virus. In embodiments, the nucleic acid sequence encoding one or more anti-cancer proteins replaces the non-essential gene of the virus. In embodiments, the non-essential gene is an E3 gene. In embodiments, the sequence encoding one or more anti-cancer proteins is operably linked to a promoter. In embodiments, the promoter is a CMV promoter.

In embodiments, the anti-cancer protein is a cytokine, a chemokine, an immune costimulatory molecule, an immune checkpoint inhibitor, a cytotoxic protein, a tumor suppressor, an apoptosis-inducing protein, or an anti-angiogenesis factor. In embodiments, the anti-cancer protein is a cytokine. In embodiments, the anti-cancer protein is a chemokine. In embodiments, the anti-cancer protein is an immune costimulatory molecule. In embodiments, the anti-cancer protein is an immune checkpoint inhibitor. In embodiments, the anti-cancer protein is a cytotoxic protein. In embodiments, the anti-cancer protein is a tumor suppressor. In embodiments, the anti-cancer protein is an apoptosis-inducing protein. In embodiments, the anti-cancer protein is an anti-angiogenesis factor.

In embodiments, the anti-cancer protein is an immune activating protein. As used herein, “immune activating protein” refers to a protein that directly or indirectly upregulates or increases production of cells or compounds (e.g. cytokines, antibodies, etc.) that stimulate an immune response (e.g. macrophage activation, cytotoxic T lymphocyte (CTL) response, a B cell response, an NK cell response, etc. or any combinations thereof). In embodiments, the immune activating protein is granulocyte-macrophage colony-stimulating factor (GM-CSF), IL-2, IL-12, IL-15, B7-1, CD137L, granulocyte colony-stimulating factor receptor (G-CSF), macrophage colony-stimulating factor (M-CSF), or an anti-CD25 antibody. In embodiments, the immune activating protein is GM-CSF. In embodiments, the immune activating protein is IL-2. In embodiments, the immune activating protein is IL-12. In embodiments, the immune activating protein is IL-15. In embodiments, the immune activating protein is B7-1. In embodiments, the immune activating protein is CD137L. In embodiments, the immune activating protein is G-CSF. In embodiments, the immune activating protein is M-CSF. In embodiments, the immune activating protein is an anti-CD25 antibody.

In embodiments, the anti-cancer protein is an apoptosis-inducing factor. As used herein, “apoptosis-inducing factor” refers to a protein that can directly or indirectly induce cell death in cancer cells or sensitize cancer cells to anti-cancer agents that cause cell death. For example, an apoptosis-inducing factor may be a protein that activates signaling pathways leading to apoptosis. In embodiments, the apoptosis-inducting factor is TNF-related apoptosis-inducing ligand (TRAIL).

In, embodiments, the anti-cancer protein is an immune checkpoint inhibitor. As used herein, “immune checkpoint inhibitor” or “checkpoint inhibitor” as provided herein refers to a substance (e.g., an antibody or fragment thereof) that is capable of inhibiting, negatively affecting (e.g., decreasing) the activity or function of a checkpoint protein (e.g., decreasing expression or decreasing the activity of a checkpoint protein) relative to the activity or function of the checkpoint protein in the absence of the inhibitor. The checkpoint inhibitor may at least in part, partially or totally block stimulation, decrease, prevent, or delay activation, or inactivate, desensitize, or down-regulate signal transduction or enzymatic activity or the amount of a checkpoint protein. A checkpoint inhibitor may inhibit a checkpoint protein, e.g., by binding, partially or totally blocking, decreasing, preventing, delaying, inactivating, desensitizing, or down-regulating activity of the checkpoint protein. In embodiments, the checkpoint inhibitor is an antibody. In embodiments, the checkpoint inhibitor is an antibody fragment. In embodiments, the checkpoint inhibitor is an antibody variant. In embodiments, the checkpoint inhibitor is a scFv. In embodiments, the immune checkpoint inhibitor is an anti-PD-1 antibody or fragment thereof, an anti-PD-L1 antibody or fragment thereof, or an anti-CTLA-4 antibody or fragment thereof. In embodiments, the immune checkpoint inhibitor is an anti-PD-1 antibody or fragment thereof. In embodiments, the immune checkpoint inhibitor is an anti-PD-L1 antibody or fragment thereof. In embodiments, the immune checkpoint inhibitor is an anti-CTLA-4 antibody or fragment thereof.

In embodiments, the MUC16 promoter includes one or more transactivation elements. As used herein, the term “transactivation element” refers to a nucleic acid sequence in a gene promoter where a trans-acting protein (e.g. a transcription factor) binds to upregulate or increase transcription of an operably-linked gene. In embodiments, the trans-acting protein is a transcription factor. In embodiments, the trans-acting protein may recruit other proteins that recruit transcriptional machinery, thereby increasing transcription of the operably-linked gene. For example, mRNA levels of the operably-linked gene (e.g. essential viral gene) may increase in the presence of a transactivation element compared to levels of mRNA in the absence of the transactivation element. In embodiments, mRNA levels of the operably-linked gene (e.g. essential viral gene) is increased 50%, 60%, 70%, 80%, 85%, 90%, 95%, 100% or more in the presence of a transactivation element compared to levels of mRNA in the absence of the transactivation element. In embodiments, mRNA levels of the operably-linked gene (e.g. essential viral gene) is increased 1×, 5×, 10×, 20×50×, 100×, 200×, 500×, 1000×, 2000×, 5000× or more in the presence of a transactivation element compared to levels of mRNA in the absence of the transactivation element. In embodiments, the MUC16 promoter does not include a transcription repression element.

For the method provided herein, in embodiments, the virus further includes a nucleic acid encoding a detectable protein. A “detectable protein” refers to a protein detectable by spectroscopic, photochemical, biochemical, immunochemical, chemical, or other physical means. In embodiments, the detectable protein is a fluorescent protein (e.g. green fluorescent protein, luciferase, etc.). In embodiments, the detectable protein is green fluorescent protein, mCherry, Emerald, or firefly luciferase. In embodiments, the detectable protein is green fluorescent protein. In embodiments, the detectable protein is mCherry. In embodiments, the detectable protein is Emerald. In embodiments, the detectable protein is firefly luciferase.

The virus may include one or more nucleic acid sequences that allows elimination of the virus (e.g. by inducing host cell death, by inhibiting viral replication, etc.) if viral replication in a host cell (e.g. CA-125 expressing cancer cell) is either insufficient or too high. For example, the virus may include a nucleic acid encoding thymidine kinase (TK). Contacting a cell infected with a virus including a TK-encoding nucleic acid with a compound such as ganciclovir, acyclovir or an analog thereof will result in elimination of the virus. For example, contacting the virus-infected cell with ganciclovir or an analog thereof can result in death of the virus-infected cell. Addition of acyclovir or an analog thereof can inhibit virus replication in the virus-infected cell. Thus, in embodiments, the virus includes a nucleic acid encoding thymidine kinase. In embodiments, the nucleic acid sequence encoding thymidine kinase forms part of a non-essential gene of the virus provided herein. In embodiments, the nucleic acid sequence encoding thymidine kinase is inserted into a non-essential gene of the virus. In embodiments, the the nucleic acid sequence encoding thymidine kinase replaces the non-essential gene of the virus. In embodiments, the non-essential gene is an E3 gene. In embodiments, the sequence encoding thymidine kinase is operably linked to a promoter. In embodiments, the promoter is a herpes simplex virus (HSV) promoter.

In embodiments, the virus is an oncolytic virus. As used herein, the term “oncolytic virus” refers to a virus that preferentially infects cancer cells relative to non-cancer cells. For example, an oncolytic virus can infect and kill the infected cancer cells by lysing the cells. Upon lysis of the cancer cell, new infectious virus particles or virions may be released to assist in destroying the remaining tumor. In embodiments, the oncolytic virus not only causes direct destruction of the tumor cells, but also stimulates host anti-tumor immune system responses. In embodiments, the virus is an oncolytic virus that preferentially infects CA-125 expressing cancer cells. In embodiments, the CA-125 expressing cancer cell expresses an elevated level of CA-125 relative to the expression level of CA-125 in a non-cancer cell. In embodiments, the CA-125 expression level of a non-cancer cell is undetectable using methods conventionally known in the art to a detect protein expression in a cell (e.g., immunofluorescent detection, protein biochemistry, RNA expression level). In embodiments, the CA-125 expression level in a non-cancer cell is about 1000, 500, 100, 50, 25, 20, 10, 5, or 1.5 times lower than the expression level of CA-125 expressing cancer cell.

In embodiments, the CA-125 expression level of a CA-125 expressing cancer cell is at least about 35 units per milliliter (U/ml). In embodiments, the CA-125 expression level of a CA-125 expressing cancer cell is at least about 40 U/ml. In embodiments, the CA-125 expression level of a CA-125 expressing cancer cell is at least about 50 U/ml. In embodiments, the CA-125 expression level of a CA-125 expressing cancer cell is at least about 60 U/ml. In embodiments, the CA-125 expression level of a CA-125 expressing cancer cell is at least about 70 U/ml. In embodiments, the CA-125 expression level of a CA-125 expressing cancer cell is at least about 80 U/ml. In embodiments, the CA-125 expression level of a CA-125 expressing cancer cell is at least about 90 U/ml. In embodiments, the CA-125 expression level of a CA-125 expressing cancer cell is at least about 100 U/ml. In embodiments, the CA-125 expression level of a CA-125 expressing cancer cell is at least about 120 U/ml. In embodiments, the CA-125 expression level of a CA-125 expressing cancer cell is at least about 140 U/ml. In embodiments, the CA-125 expression level of a CA-125 expressing cancer cell is at least about 160 U/ml. In embodiments, the CA-125 expression level of a CA-125 expressing cancer cell is at least about 180 U/ml. In embodiments, the CA-125 expression level of a CA-125 expressing cancer cell is at least about 200 U/ml. In embodiments, the CA-125 expression level of a CA-125 expressing cancer cell is at least about 300 U/ml. In embodiments, the CA-125 expression level of a CA-125 expressing cancer cell is at least about 400 U/ml. In embodiments, the CA-125 expression level of a CA-125 expressing cancer cell is at least about 500 U/ml. In embodiments, the CA-125 expression level of a CA-125 expressing cancer cell is at least about 600 U/ml. In embodiments, the CA-125 expression level of a CA-125 expressing cancer cell is at least about 700 U/ml. In embodiments, the CA-125 expression level of a CA-125 expressing cancer cell is at least about 800 U/ml. In embodiments, the CA-125 expression level of a CA-125 expressing cancer cell is at least about 900 U/ml. In embodiments, the CA-125 expression level of a CA-125 expressing cancer cell is at least about 1000 U/ml.

In embodiments, the CA-125 expression level of a CA-125 expressing cancer cell is about 35 units per milliliter (U/ml). In embodiments, the CA-125 expression level of a CA-125 expressing cancer cell is about 40 U/ml. In embodiments, the CA-125 expression level of a CA-125 expressing cancer cell is about 50 U/ml. In embodiments, the CA-125 expression level of a CA-125 expressing cancer cell is about 60 U/ml. In embodiments, the CA-125 expression level of a CA-125 expressing cancer cell is about 70 U/ml. In embodiments, the CA-125 expression level of a CA-125 expressing cancer cell is about 80 U/ml. In embodiments, the CA-125 expression level of a CA-125 expressing cancer cell is about 90 U/ml. In embodiments, the CA-125 expression level of a CA-125 expressing cancer cell is about 100 U/ml. In embodiments, the CA-125 expression level of a CA-125 expressing cancer cell is about 120 U/ml. In embodiments, the CA-125 expression level of a CA-125 expressing cancer cell is about 140 U/ml. In embodiments, the CA-125 expression level of a CA-125 expressing cancer cell is about 160 U/ml. In embodiments, the CA-125 expression level of a CA-125 expressing cancer cell is about 180 U/ml. In embodiments, the CA-125 expression level of a CA-125 expressing cancer cell is about 200 U/ml. In embodiments, the CA-125 expression level of a CA-125 expressing cancer cell is about 300 U/ml. In embodiments, the CA-125 expression level of a CA-125 expressing cancer cell is about 400 U/ml. In embodiments, the CA-125 expression level of a CA-125 expressing cancer cell is about 500 U/ml. In embodiments, the CA-125 expression level of a CA-125 expressing cancer cell is about 600 U/ml. In embodiments, the CA-125 expression level of a CA-125 expressing cancer cell is about 700 U/ml. In embodiments, the CA-125 expression level of a CA-125 expressing cancer cell is about 800 U/ml. In embodiments, the CA-125 expression level of a CA-125 expressing cancer cell is about 900 U/ml. In embodiments, the CA-125 expression level of a CA-125 expressing cancer cell is about 1000 U/ml.

In embodiments, the CA-125 expression level of a non-cancer cell is less than about 35 U/mL. In embodiments, the CA-125 expression level of a non-cancer cell is less than about 30 U/ml. In embodiments, the CA-125 expression level of a non-cancer cell is less than about 25 U/ml. In embodiments, the CA-125 expression level of a non-cancer cell is less than about 20 U/ml. In embodiments, the CA-125 expression level of a non-cancer cell is less than about 15 U/ml. In embodiments, the CA-125 expression level of a non-cancer cell is less than about 10 U/ml. In embodiments, the CA-125 expression level of a non-cancer cell is less than about 5 U/ml. In embodiments, the CA-125 expression level of a non-cancer cell is less than about 2.5 U/ml. In embodiments, the CA-125 expression level of a non-cancer cell is less than about 1 U/ml. In embodiments, the CA-125 expression level of a non-cancer cell is less than about 0.5 U/ml. In embodiments, the CA-125 expression level of a non-cancer cell is less than about 0.1 U/ml.

In embodiments, the CA-125 expression level of a non-cancer cell is about 30 U/ml. In embodiments, the CA-125 expression level of a non-cancer cell is about 25 U/ml. In embodiments, the CA-125 expression level of a non-cancer cell is about 20 U/ml. In embodiments, the CA-125 expression level of a non-cancer cell is about 15 U/ml. In embodiments, the CA-125 expression level of a non-cancer cell is about 10 U/ml. In embodiments, the CA-125 expression level of a non-cancer cell is about 5 U/ml. In embodiments, the CA-125 expression level of a non-cancer cell is about 2.5 U/ml. In embodiments, the CA-125 expression level of a non-cancer cell is about 1 U/ml. In embodiments, the CA-125 expression level of a non-cancer cell is about 0.5 U/ml. In embodiments, the CA-125 expression level of a non-cancer cell is about 0.1 U/ml. In embodiments, the CA-125 expression level of a non-cancer cell is undetectable.

The term “essential viral gene” as used herein in relation to the virus provided herein, refers to a gene required for one or more of viral replication, viral amplification or viral spread (e.g. transmission of the virus from one cell to another cell). Thus, in embodiments, an essential viral gene is a gene required for viral propagation. In embodiments, the essential viral gene is a gene essential for viral replication. In embodiments, the essential viral gene is a gene essential for viral amplification. In embodiments, the essential viral gene is a gene essential for viral spread. For example, the essential viral gene may be essential for virus entry into a host cell. In another example, the essential viral gene may be a gene required for viral DNA replication. In another example, an essential viral gene may be a gene required for transcription of viral RNA to mRNA. For example, deletion of an essential viral gene can decrease viral replication by at least 70%, 75%, 80%, 85%, 90%, 95% or more in comparison to a control including expression of the essential gene. For example, deletion of an essential viral gene can decrease viral amplification by at least 70%, 75%, 80%, 85%, 90%, 95% or more in comparison to a control including expression of the essential gene. For example, deletion of an essential viral gene can decrease viral spread (e.g. transmission of the virus from one cell to another cell) by at least 70%, 75%, 80%, 85%, 90%, 95% or more in comparison to a control including expression of the essential gene. In embodiments, the essential viral gene is an essential adenovirus gene. In embodiments, the essential viral gene is E1A. The E1A gene encodes for proteins required for efficient viral replication; thus, disruption of this gene can severely restrict efficient viral replication. In embodiments, the essential viral gene is E1B. In embodiments, the essential viral gene is E2A. In embodiments, the essential viral gene is E2B.

In embodiments, the MUC16 promoter replaces the E1A promoter in the virus genome (e.g. adenovirus genome). In embodiments, the MUC16 promoter partially or completely replaces the E1A promoter in the virus genome. In embodiments, the MUC16 promoter partially or completely replaces the E1A promoter in an adenovirus construct (e.g. an adenovirus vector). In embodiments, the MUC16 promoter replaces the E1B promoter in the virus genome (e.g. adenovirus genome). In embodiments, the MUC16 promoter partially or completely replaces the E1B promoter in the virus genome. In embodiments, the MUC16 promoter partially or completely replaces the E1B promoter in an adenovirus construct (e.g. an adenovirus vector). In embodiments, the MUC16 promoter replaces the E2A promoter in the virus genome (e.g. adenovirus genome). In embodiments, the MUC16 promoter partially or completely replaces the E2A promoter in the virus genome. In embodiments, the MUC16 promoter partially or completely replaces the E2A promoter in an adenovirus construct (e.g. an adenovirus vector). In embodiments, the MUC16 promoter replaces the E2B promoter in the virus genome (e.g. adenovirus genome). In embodiments, the MUC16 promoter partially or completely replaces the E2B promoter in the virus genome. In embodiments, the MUC16 promoter partially or completely replaces the E2B promoter in an adenovirus construct (e.g. adenovirus vector).

For the method provided herein, in embodiments, the virus is an adenovirus, a herpes simplex virus, a vaccinia virus, a retrovirus, a measles virus, a reovirus, a coxsackievirus, a poliovirus, a Newcastle disease virus, a vesicular stomatitis virus, a Zika virus, an influenza virus, a rhinovirus, or a parvovirus. In embodiments, the virus is a herpes simplex virus. In embodiments, the virus is a vaccinia virus. In embodiments, the virus is a retrovirus. In embodiments, the virus is a measles virus. In embodiments, the virus is a reovirus. In embodiments, the virus is a coxsackievirus. In embodiments, the virus is a poliovirus. In embodiments, the virus is a Newcastle disease virus. In embodiments, the virus is a vesicular stomatitis virus. In embodiments, the virus is a Zika virus. In embodiments, the virus is an influenza virus. In embodiments, the virus is a rhinovirus. In embodiments, the virus is a parvovirus. In embodiments, the virus is an adenovirus. In embodiments, the virus is a chimeric poxvirus. Chimeric poxviruses are discussed in greater detail in WO 2018/031694, the content of which is incorporated herein by reference in its entirety for all purposes.

Adenoviruses are medium-sized (90-100 nm), non-enveloped (naked), icosahedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5 (Ad5), 6, 7, 8, 9, 10, 11 (Ad11A and Ad 11P), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. In embodiments, the adenovirus is human adenovirus type 5 (Ad5).

### Methods of Making

In an aspect is provided a virus as provided herein including embodiments thereof, wherein the virus is formed by a method including: i) contacting a cell with a nucleic acid encoding an essential viral gene operably linked to the MUC16 promoter; and ii) allowing the cell to express the essential viral gene. In embodiments, the contacting a cell with the nucleic acid of step i) includes delivering the nucleic acid provided herein including embodiments thereof into the cell. In embodiments, the nucleic acid is delivered by a virus like particle (VLP) or a virus. In embodiments, the nucleic acid is part of an adenovirus vector. In embodiments, the cell is a CA-125 expressing cancer cell. In embodiments, the CA-125 expressing cancer cell is from a mammal. In embodiments, the CA-125 expressing cancer cell is from a human.

### Nucleic Acids

The compositions provided herein include nucleic acid molecules encoding the virus provided herein including embodiments thereof. The virus encoded by the nucleic acid is described in detail throughout this application (including the description above and in the examples section). Thus, in an aspect is provided an isolated nucleic acid encoding a virus provided herein including embodiments thereof.

The nucleic acid provided herein may form part of a vector. Thus, in another aspect is provided a vector including the nucleic acid provided herein including embodiments thereof. In embodiments, the vector is a viral vector. In embodiments, the viral vector is an adenovirus vector.

### Pharmaceutical Compositions

The compositions provided herein include pharmaceutical compositions including the virus provided herein including embodiments thereof. Thus, in an aspect is provided a pharmaceutical composition including a therapeutically effective amount of an virus provided herein including embodiments thereof. In another aspect is provided a pharmaceutical composition including a therapeutically effective amount of an virus provided herein including embodiments thereof and a pharmaceutically effective excipient.

The pharmaceutical composition provided herein may include a combination of, for example, two, three, or more of the viruses as described herein. For example, the composition may comprise a first virus including a first nucleic acid encoding a first anti-cancer protein and a second virus including a second nucleic acid encoding a second anti-cancer protein. In embodiments, the first anti-cancer protein and the second anti-cancer protein may have different anti-cancer activities (e.g. an apoptosis-inducing function and an immune-activating function, etc.). In embodiments, the first anti-cancer protein and the second anti-cancer protein (e.g. GM-CSF and IL-2, etc.) may function on different phases of the immune response. For example, the first anti-cancer protein may stimulate production or maturation of immune cells and the second anti-cancer protein may increase cell killing activity of immune cells (e.g. NK cells, cytotoxic T cells, etc.). In another example, the composition may comprise a first virus including a first nucleic acid encoding a plurality of anti-cancer proteins and a second virus including a second nucleic acid encoding a plurality of anti-cancer proteins. In embodiments, the first nucleic acid and the second nucleic acid encode different anti-cancer proteins. Thus, the first nucleic acid and second nucleic acid may include may include sequences encoding a combination of anti-cancer proteins provided herein including embodiments thereof. The combination of anti-cancer proteins encoded by the first and second nucleic acids are contemplated to maximize anti-cancer activity (e.g. inhibiting cancer cell proliferation, killing cancer cells, etc.) of the viruses. Thus, in embodiments, the pharmaceutical composition includes a first virus and a second virus as provided herein including embodiments thereof. In embodiments, the first virus includes a first nucleic acid sequence encoding one or more anti-cancer protein and the second virus includes a second nucleic acid sequence encoding one or more anti-cancer proteins.

The pharmaceutical composition may further include a third virus, wherein the third virus includes a third nucleic acid sequence encoding a third anti-cancer protein. In embodiments, the third nucleic acid encodes one or more anti-cancer proteins provided herein including embodiments thereof. Thus, in embodiments, the pharmaceutical composition includes a third virus. In embodiments, the third virus includes a third nucleic acid sequence encoding one or more anti-cancer proteins. In embodiments, the first nucleic acid, the second nucleic acid, and the third nucleic acid encode different anti-cancer proteins.

In embodiments, the first nucleic, the second nucleic acid, and the third nucleic acid independently encode a combination of one, two or three anti-cancer proteins as provided herein including embodiments thereof. In embodiments, the first nucleic, the second nucleic acid, and the third nucleic acid independently encode GM-CSF, CD137L, IL-2, IL-12, IL-15, TRAIL, or combinations thereof. In embodiments, the first nucleic encodes GM-CSF, CD137L, IL-2, IL-12, IL-15, TRAIL, or combinations thereof. In embodiments, the first nucleic encodes GM-CSF. In embodiments, the first nucleic encodes CD137L. In embodiments, the first nucleic encodes IL-2. In embodiments, the first nucleic encodes IL-12. In embodiments, the first nucleic encodes IL-15. In embodiments, the first nucleic encodes TRAIL.

In embodiments, the second nucleic encodes GM-CSF, CD137L, IL-2, IL-12, IL-15, TRAIL, or combinations thereof. In embodiments, the second nucleic encodes GM-CSF. In embodiments, the second nucleic encodes CD137L. In embodiments, the second nucleic encodes IL-2. In embodiments, the second nucleic encodes IL-12. In embodiments, the second nucleic encodes IL-15. In embodiments, the second nucleic encodes TRAIL.

In embodiments, the third nucleic encodes GM-CSF, CD137L, IL-2, IL-12, IL-15, TRAIL, or combinations thereof. In embodiments, the third nucleic encodes GM-CSF. In embodiments, the third nucleic encodes CD137L. In embodiments, the third nucleic encodes IL-2. In embodiments, the third nucleic encodes IL-12. In embodiments, the third nucleic encodes IL-15. In embodiments, the third nucleic encodes TRAIL.

### Cell Compositions

The virus provided herein including embodiments thereof selectively replicates in CA-125 expressing cancer cells (e.g. ovarian cancer, uterine cancer, or cervical cancer), thereby allowing specific cancer cell targeting. Thus, in an aspect is provided a cell including the virus provided herein including embodiments thereof.

In embodiments, the cell is a CA-125 expressing cancer cell. In embodiments, the CA-125 expressing cancer cell is an ovarian cancer cell, a uterine cancer cell, or a cervical cancer cell. In embodiments, the CA-125 expressing cancer cell expresses a higher level of CA-125 as compared to the standard control (e.g. a non-cancer cell, a CA-125 negative cell). In embodiments, the expression level of CA-125 in a CA-125 expressing cancer cell is 1.5, 5, 10, 20, 25, 50, 100, 500 or 1000 times higher than the expression level of a standard control (e.g. a non-cancer cell, a CA-125 negative cell). Detection levels of CA-125 may be assessed using conventional methods known in the art (e.g., immunofluorescent detection, protein biochemistry, RNA expression level).

### Methods of Use

The virus provided herein, including embodiments thereof is contemplated to be effective for treating and/or preventing MUC16 expressing cancers. The virus provided herein is contemplated to be a potent targeted agent that can not only specifically replicate in and lyse CA-125 expressing cancer cells (e.g. ovarian cancer cells), but can further activate a protective anti-cancer immune response. Thus, in an aspect is provided a method of treating or preventing cancer in a subject in need thereof, the method including administering to the subject a therapeutically or prophylactically effective amount of the virus provided herein including embodiments thereof. In embodiments, the method includes administering to the subject a therapeutically effective amount of the virus. In embodiments, the method includes administering to the subject a prophylactically effective amount of the virus.

In embodiments, the cancer is a gynecological cancer. In embodiments, the cancer is ovarian cancer, uterine cancer, or cervical cancer. In embodiments, the cancer is ovarian cancer. In embodiments, the cancer is uterine cancer. In embodiments, the cancer is cervical cancer. In embodiments, the cancer expresses CA-125 (e.g. a CA-125 expressing cancer).

The virus provided herein including embodiments thereof is contemplated to be effective as a cancer vaccine that can be administered to a patient who already suffers from cancer (e.g. a CA-125 expressing cancer) or who previously had the cancer. In some examples, the virus exhibits anti-cancer activity, e.g. reduction of cancer cell number, reduction of cancer size, killing of cancer cells, reductions and/or inhibition of metastasis and reduction of cancer cell growth and/or proliferation. In other examples, the virus provided herein can also be used for a prophylactic purposes, especially in a subject who is considered predisposed for a cancer (e.g. a gynecological cancer, a CA-125 expressing cancer), but presently does not have the cancer. The virus can be therefore be administered to the predisposed subject, for preventing or reducing a likelihood of the occurrence of the cancer in the subject. In another example, the virus can be administered to a subject who previously had the CA-125 expressing cancer, but presently does not have the cancer. The virus can therefore be administered to the subject to prevent recurrence of the cancer.

It is contemplated that the virus provided herein including embodiments thereof induces an anti-cancer immune response upon infection of a cancer cell. For example, the virus may indirectly induce an immune response by infection of cancer cells. For example, the virus provided herein induces an immune response by expression of one or more immunomodulatory proteins (e.g. a cytokine, a chemokine, an immune costimulatory molecule, an immune checkpoint inhibitor, a cytotoxic protein, an immune-activating protein, etc). Thus, in an aspect is provided a method of stimulating an immune response in a subject in need thereof, the method including administering to the subject an effective amount of the virus provided herein including embodiments thereof. In embodiments, the subject has or previously had a CA-125 expressing cancer.

In one embodiment, a response rate to a composition (e.g. the virus provided herein), can be compared to a baseline reference or control reference. The term “response rate” is used herein in its customary sense to indicate the percentage of patients who respond with cancer recession following treatment. Response rates include, for example, partial or complete recession. A partial response includes an about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, or about 99% recession of cancer cells. In some embodiments, the control reference is obtained from a healthy subject, a cancer subject (e.g., the cancer subject being treated or another cancer subject), or any population thereof.

For the method provided herein, in embodiments, the virus is administered at an amount from about 10⁷ to about 10¹⁴ plaque forming units (Pfu). In embodiments, the virus is administered at an amount from about 10¹⁰ to about 10¹² plaque forming units (Pfu). In embodiments, the virus is administered at an amount of about 10⁷ Pfu. In embodiments, the virus is administered at an amount of about 10⁸ Pfu. In embodiments, the virus is administered at an amount of about 10⁹ Pfu. In embodiments, the virus is administered at an amount of about 10¹⁰ Pfu. In embodiments, the virus is administered at an amount of about 10¹¹ Pfu. In embodiments, the virus is administered at an amount of about 10¹² Pfu. In embodiments, the virus is administered at an amount of about 10¹³ Pfu. In embodiments, the virus is administered at an amount of about 10¹⁴ Pfu.

In embodiments, the virus is administered intraperitoneally, intratumorally, intravenously, intrathecally, or intrapleurally. In embodiments, the virus is administered intraperitoneally. In embodiments, the virus is administered intratumorally. In embodiments, the virus is administered intravenously. In embodiments, the virus is administered intrathecally. In embodiments, the virus is administered intrapleurally.

In instances, a cell comprising the virus provided herein (e.g. a virus-infected cell) is contemplated to be effective for treating or preventing cancer. Applicant has demonstrated herein that virus-infected cells can be administered to a subject to effectively inhibit cancer cell growth and prolong survival in a subject. In embodiments, the virus-infected cells may be administered directly to the tumor microenvironment or to the peritoneum, wherein the virus is released to infect cancer cells. Thus, in an aspect is provided a method of treating or preventing cancer in a subject in need thereof, the method including administering to the subject a therapeutically or prophylactically effective amount of a cell including the virus provided herein including embodiments thereof. In embodiments, a therapeutically effective amount of a cell including the virus is administered to the subject. In embodiments, a prophylactically effective amount of a cell including the virus is administered to the subject.

In embodiments, the cell including the virus is a CA-125 expressing cancer cell (e.g. an ovarian cancer cell, a uterine cancer cell, a cervical cancer cell). In embodiments, the CA-125 expressing cancer cell is obtained from the subject. For example, the CA-125 expressing cancer cell may be taken from the subject, infected with the virus, and administered back to the patient. For example, the CA-125 cancer cell may be taken from ascites or a solid tumor from the subject and subsequently contacted with the virus, thereby resulting in a virus-infected cell. In embodiments, the cell including the virus (virus-infected cell) provided herein is administered back to the subject intraperitoneally, intratumorally, intravenously, intrathecally, or intrapleurally. In embodiments, the cell is administered intraperitoneally.

In embodiments, a cell including the virus provided herein (e.g. a virus-infected cell) is made by contacting the cell (e.g. CA-125 expressing cancer cell) with the virus at an multiplicity of infection (MOI) of 0.1:1 to 20:1. In embodiments, the cell is contacted with the virus at an MOI of 0.5:1 to 20:1. In embodiments, the cell is contacted with the virus at an MOI of 1:1 to 20:1. In embodiments, the cell is contacted with the virus at an MOI of 1.5:1 to 20:1. In embodiments, the cell is contacted with the virus at an MOI of 2:1 to 20:1. In embodiments, the cell is contacted with the virus at an MOI of 2.5:1 to 20:1. In embodiments, the cell is contacted with the virus at an MOI of 3:1 to 20:1. In embodiments, the cell is contacted with the virus at an MOI of 3.5:1 to 20:1. In embodiments, the cell is contacted with the virus at an MOI of 4:1 to 20:1. In embodiments, the cell is contacted with the virus at an MOI of 4.5:1 to 20:1. In embodiments, the cell is contacted with the virus at an MOI of 5:1 to 20:1. In embodiments, the cell is contacted with the virus at an MOI of 5.5:1 to 20:1. In embodiments, the cell is contacted with the virus at an MOI of 6:1 to 20:1. In embodiments, the cell is contacted with the virus at an MOI of 6.5:1 to 20:1. In embodiments, the cell is contacted with the virus at an MOI of 7:1 to 20:1. In embodiments, the cell is contacted with the virus at an MOI of 7.5:1 to 20:1. In embodiments, the cell is contacted with the virus at an MOI of 8:1 to 20:1. In embodiments, the cell is contacted with the virus at an MOI of 8.5:1 to 20:1. In embodiments, the cell is contacted with the virus at an MOI of 9:1 to 20:1. In embodiments, the cell is contacted with the virus at an MOI of 9.5:1 to 20:1. In embodiments, the cell is contacted with the virus at an MOI of 10:1 to 20:1. In embodiments, the cell is contacted with the virus at an MOI of 10.5:1 to 20:1. In embodiments, the cell is contacted with the virus at an MOI of 11:1 to 20:1. In embodiments, the cell is contacted with the virus at an MOI of 11.5:1 to 20:1. In embodiments, the cell is contacted with the virus at an MOI of 12:1 to 20:1. In embodiments, the cell is contacted with the virus at an MOI of 12.5:1 to 20:1. In embodiments, the cell is contacted with the virus at an MOI of 13:1 to 20:1. In embodiments, the cell is contacted with the virus at an MOI of 13.5:1 to 20:1. In embodiments, the cell is contacted with the virus at an MOI of 14:1 to 20:1. In embodiments, the cell is contacted with the virus at an MOI of 14.5:1 to 20:1. In embodiments, the cell is contacted with the virus at an MOI of 15:1 to 20:1. In embodiments, the cell is contacted with the virus at an MOI of 15.5:1 to 20:1. In embodiments, the cell is contacted with the virus at an MOI of 16:1 to 20:1. In embodiments, the cell is contacted with the virus at an MOI of 16.5:1 to 20:1. In embodiments, the cell is contacted with the virus at an MOI of 17:1 to 20:1. In embodiments, the cell is contacted with the virus at an MOI of 17.5:1 to 20:1. In embodiments, the cell is contacted with the virus at an MOI of 18:1 to 20:1. In embodiments, the cell is contacted with the virus at an MOI of 18.5:1 to 20:1. In embodiments, the cell is contacted with the virus at an MOI of 19:1 to 20:1. In embodiments, the cell is contacted with the virus at an MOI of 19.5:1 to 20:1.

In embodiments, the cell is contacted with the virus at an MOI of 0.1:1 to 19.5:1. In embodiments, the cell is contacted with the virus at an MOI of 0.1:1 to 19:1. In embodiments, the cell is contacted with the virus at an MOI of 0.1:1 to 18.5:1. In embodiments, the cell is contacted with the virus at an MOI of 0.1:1 to 18:1. In embodiments, the cell is contacted with the virus at an MOI of 0.1:1 to 17.5:1. In embodiments, the cell is contacted with the virus at an MOI of 0.1:1 to 17:1. In embodiments, the cell is contacted with the virus at an MOI of 0.1:1 to 16.5:1. In embodiments, the cell is contacted with the virus at an MOI of 0.1:1 to 16:1. In embodiments, the cell is contacted with the virus at an MOI of 0.1:1 to 15.5:1. In embodiments, the cell is contacted with the virus at an MOI of 0.1:1 to 15:1. In embodiments, the cell is contacted with the virus at an MOI of 0.1:1 to 14.5:1. In embodiments, the cell is contacted with the virus at an MOI of 0.1:1 to 14:1. In embodiments, the cell is contacted with the virus at an MOI of 0.1:1 to 13.5:1. In embodiments, the cell is contacted with the virus at an MOI of 0.1:1 to 13:1. In embodiments, the cell is contacted with the virus at an MOI of 0.1:1 to 12.5:1. In embodiments, the cell is contacted with the virus at an MOI of 0.1:1 to 12:1. In embodiments, the cell is contacted with the virus at an MOI of 0.1:1 to 11.5:1. In embodiments, the cell is contacted with the virus at an MOI of 0.1:1 to 11:1. In embodiments, the cell is contacted with the virus at an MOI of 0.1:1 to 10.5:1. In embodiments, the cell is contacted with the virus at an MOI of 0.1:1 to 10:1. In embodiments, the cell is contacted with the virus at an MOI of 0.1:1 to 9.5:1. In embodiments, the cell is contacted with the virus at an MOI of 0.1:1 to 9:1. In embodiments, the cell is contacted with the virus at an MOI of 0.1:1 to 8.5:1. In embodiments, the cell is contacted with the virus at an MOI of 0.1:1 to 8:1. In embodiments, the cell is contacted with the virus at an MOI of 0.1:1 to 7.5:1. In embodiments, the cell is contacted with the virus at an MOI of 0.1:1 to 7:1. In embodiments, the cell is contacted with the virus at an MOI of 0.1:1 to 6.5:1. In embodiments, the cell is contacted with the virus at an MOI of 0.1:1 to 6:1. In embodiments, the cell is contacted with the virus at an MOI of 0.1:1 to 5.5:1. In embodiments, the cell is contacted with the virus at an MOI of 0.1:1 to 5:1. In embodiments, the cell is contacted with the virus at an MOI of 0.1:1 to 4.5:1. In embodiments, the cell is contacted with the virus at an MOI of 0.1:1 to 4:1. In embodiments, the cell is contacted with the virus at an MOI of 0.1:1 to 3.5:1. In embodiments, the cell is contacted with the virus at an MOI of 0.1:1 to 3:1. In embodiments, the cell is contacted with the virus at an MOI of 0.1:1 to 2.5:1. In embodiments, the cell is contacted with the virus at an MOI of 0.1:1 to 2:1. In embodiments, the cell is contacted with the virus at an MOI of 0.1:1 to 1.5:1. In embodiments, the cell is contacted with the virus at an MOI of 0.1:1 to 1:1. In embodiments, the cell is contacted with the virus at an MOI of 0.1:1 to 0.5:1. In embodiments, the cell is contacted with the virus at an MOI of 0.1:1, 0.5:1, 1:1, 1.5:1, 2:1:1, 2.5:1, 3:1, 3.5:1, 4:1, 4.5:1, 5, 5.5:1, 6:1, 6.5:1, 7:1, 7.5:1, 8:1, 8.5:1, 9, 9.5:1, 10:1, 10.5:1, 11:1, 11.5:1, 12:1, 12.5:1, 13:1, 13.5:1, 14:1, 14.5:1, 15:1, 15.5:1, 16:1, 16.5:1, 17:1, 17.5:1, 18:1, 18.5:1, 19:1, 19.5:1, or 20:1. In embodiments, the cell is contacted with the virus at an MOI of 10:1. In embodiments, the cell is contacted with the virus at an MOI of 20:1.

In embodiments, the cell including the virus provided herein (e.g. a virus-infected cell) is made by contacting a cell (e.g. CA-125 expressing cancer cell) with the virus at an multiplicity of infection (MOI) of 1:1 to 120:1. In embodiments, the cell is contacted with the virus at an MOI of 10:1 to 120:1. In embodiments, the cell is contacted with the virus at an MOI of 20:1 to 120:1. In embodiments, the cell is contacted with the virus at an MOI of 30:1 to 120:1. In embodiments, the cell is contacted with the virus at an MOI of 40:1 to 120:1. In embodiments, the cell is contacted with the virus at an MOI of 50:1 to 120:1. In embodiments, the cell is contacted with the virus at an MOI of 60:1 to 120:1. In embodiments, the cell is contacted with the virus at an MOI of 70:1 to 120:1. In embodiments, the cell is contacted with the virus at an MOI of 80:1 to 120:1. In embodiments, the cell is contacted with the virus at an MOI of 90:1 to 120:1. In embodiments, the cell is contacted with the virus at an MOI of 100:1 to 120:1. In embodiments, the cell is contacted with the virus at an MOI of 110:1 to 120:1.

In embodiments, the cell is contacted with the virus at an MOI of 1:1 to 110:1. In embodiments, the cell is contacted with the virus at an MOI of 1:1 to 100:1. In embodiments, the cell is contacted with the virus at an MOI of 1:1 to 90:1. In embodiments, the cell is contacted with the virus at an MOI of 1:1 to 80:1. In embodiments, the cell is contacted with the virus at an MOI of 1:1 to 70:1. In embodiments, the cell is contacted with the virus at an MOI of 1:1 to 60:1. In embodiments, the cell is contacted with the virus at an MOI of 1:1 to 50:1. In embodiments, the cell is contacted with the virus at an MOI of 1:1 to 40:1. In embodiments, the cell is contacted with the virus at an MOI of 1:1 to 30:1. In embodiments, the cell is contacted with the virus at an MOI of 1:1 to 20:1. In embodiments, the cell is contacted with the virus at an MOI of 1:1 to 10:1. In embodiments, the cell is contacted with the virus at an MOI of 1:1, 10:1, 20:1, 30:1, 40:1, 50:1, 60:1, 70:1, 80:1, 90:1, 100:1, 110:1, or 120:1. In embodiments, the cell is contacted with the virus at an MOI of 20:1. In embodiments, the cell is contacted with the virus at an MOI of 100:1.

A cell including the virus provided herein including embodiments thereof is contemplated to be effective as a vaccine that can be used to stimulate an anti-cancer immune response to eradicate cancer cells. Thus, in an aspect is provided a method of stimulating an immune response in a subject in need thereof, the method including administering to the subject an effective amount of a cell including the virus provided herein including embodiments thereof. In embodiments, the subject has or previously had a CA-125 expressing cancer. In embodiments, the cell is a CA-125 expressing cancer cell. As described above and throughout the specification, the cell may be a CA-125 expressing cancer cell obtained from the subject. For example, the CA-125 expressing cell may be a CA-125 expressing cancer cell obtained from ascites or from a solid tumor from the subject. The CA-125 expressing cancer cell may be taken from the subject, infected with the virus, and administered back to the subject.

The virus provided herein including embodiments thereof are effective for inhibiting proliferation of a CA-125 expressing cell. The virus may prevent CA-125 cell proliferation through oncolytic activity or through an anti-cancer immune response. For example, virus infection of a CA-125 expressing cell may result in cancer antigen presentation, production of cytokines, and/or activation of immune cells. Thus, in an aspect is provided a method of inhibiting proliferation of a CA-125 expressing cell, the method including contacting the CA-125 expressing cell with a virus provided herein including embodiments thereof. In embodiments, the method includes inducing apoptosis of the CA-125 expressing cell. In embodiments, the CA-125 expressing cell is in a subject having cancer. In embodiments, cancer is a gynecological cancer. In embodiments, the cancer is ovarian cancer, uterine cancer, or cervical cancer. In embodiments, the cancer is ovarian cancer. In embodiments, the cancer is uterine cancer. In embodiments, the cancer is cervical cancer. In embodiments, the CA-125 expressing cell is a cancer cell.

In an another aspect is provided a method of detecting a CA-125 expressing cancer in a subject in need thereof, the method including contacting a CA-125 expressing cancer cell with a virus as described herein including embodiments thereof. In embodiments, the virus includes a nucleic acid encoding a detectable protein. In embodiments, the detectable protein is a fluorescent protein. In embodiments, the detectable protein is mCherry. In embodiments, the the detectable protein is Emerald. In embodiments, the detectable the detectable protein is firefly luciferase.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EMBODIMENTS

Embodiment 1. A virus comprising a nucleic acid encoding a MUC16 promoter operably linked to an essential viral gene.

Embodiment 2. The virus of embodiment 1, wherein said MUC16 promoter is from about 20 nucleic acid residues to about 6000 nucleic acid residues in length.

Embodiment 3. The virus of embodiment 1 or 2, wherein said MUC16 promoter is from about 800 nucleic acid residues to about 1200 nucleic acid residues in length.

Embodiment 4. The virus of embodiment 1 or 2, wherein said MUC16 promoter is from about 400 nucleic acid residues to about 800 nucleic acid residues in length.

Embodiment 5. The virus of embodiment 1, wherein said MUC16 promoter comprises a nucleic acid sequence within a region from about −6000 nucleic acid residues upstream to about +200 nucleic residues downstream of a MUC16 transcription start site (TSS).

Embodiment 6. The virus of embodiment 5, wherein said nucleic acid sequence is from about −1200 nucleic acid residues upstream to about +100 nucleic acid residues downstream of the MUC16 TSS.

Embodiment 7. The virus of embodiment 5, wherein said nucleic acid sequence is from about −1200 nucleic acid residues upstream to about −500 nucleic residues upstream of the MUC16 TSS.

Embodiment 8. The virus of any one of embodiments 1-3, wherein said MUC16 promoter comprises the nucleic acid sequence of SEQ ID NO:1.

Embodiment 9. The virus of any one of embodiments 1, 2, or 4, wherein said MUC16 promoter comprises the nucleic acid sequence of SEQ ID NO:2.

Embodiment 10. The virus of any one of embodiments 1-9, wherein said virus further includes a nucleic acid sequence encoding one or more anti-cancer proteins.

Embodiment 11. The virus of embodiment 10, wherein said anti-cancer protein is a cytokine, a chemokine, an immune costimulatory molecule, an immune checkpoint inhibitor, a cytotoxic protein, a tumor suppressor, an apoptosis-inducing protein, or an anti-angiogenesis factor.

Embodiment 12. The virus of embodiment 10 or 11, wherein said anti-cancer protein is an immune-activating protein.

Embodiment 13. The virus of embodiment 12, wherein said immune-activating protein is granulocyte-macrophage colony-stimulating factor (GM-CSF), IL-2, IL-12, IL-15, B7-1, CD137L, granulocyte colony-stimulating factor receptor (G-CSF), macrophage colony-stimulating factor (M-CSF), or an anti-CD25 antibody.

Embodiment 14. The virus of embodiment 12, wherein said immune-activating protein is GM-CSF.

Embodiment 15. The virus of embodiment 10 or 11, wherein said anti-cancer protein is an apoptosis-inducing factor.

Embodiment 16. The virus of embodiment 15, wherein said apoptosis-inducting factor is TNF-related apoptosis-inducing ligand (TRAIL).

Embodiment 17. The virus of embodiment 10 or 11, where in the anti-cancer protein is an immune checkpoint inhibitor.

Embodiment 18. The virus of embodiment 17, wherein the immune checkpoint inhibitor is an anti-PD-1 antibody, an anti-PD-L1 antibody, or an anti-CTLA-4 antibody.

Embodiment 19. The virus of any one of embodiments 1-18, wherein said MUC16 promoter comprises one or more transactivation elements.

Embodiment 20. The virus of any one of embodiments 1-19, further comprising a nucleic acid encoding a detectable protein.

Embodiment 21. The virus of embodiment 20, wherein said detectable protein is a fluorescent protein.

Embodiment 22. The virus of any one of embodiments 1-21, further comprising a nucleic acid encoding thymidine kinase.

Embodiment 23. The virus of any one of embodiments 1-22, wherein said virus is an oncolytic virus.

Embodiment 24. The virus of any one of embodiments 1-23, wherein said essential viral gene is E1A.

Embodiment 25. The virus of any one of embodiments 1-24, wherein said virus is an adenovirus, a herpes simplex virus, a vaccinia virus, a retrovirus, a measles virus, a reovirus, a coxsackievirus, a poliovirus, a Newcastle disease virus, a vesicular stomatitis virus, a Zika virus, an influenza virus, a rhinovirus, or a parvovirus.

Embodiment 26. The virus of any one of embodiments 1-25, wherein said virus is an adenovirus.

Embodiment 27. The virus of any one of embodiments 1-26, wherein said virus is formed by a method comprising: i) contacting a cell with a nucleic acid encoding essential viral genes and the MUC16 promoter; and ii) allowing said cell to express said essential viral genes.

Embodiment 28. The virus of embodiment 27, wherein said cell is a CA-125 expressing cancer cell.

Embodiment 29. An isolated nucleic acid encoding the virus of any one of embodiments 1-26.

Embodiment 30. A pharmaceutical composition comprising a therapeutically effective amount of the virus of any one of embodiments 1-26.

Embodiment 31. The pharmaceutical composition of embodiment 30, comprising a first virus and a second virus.

Embodiment 32. The pharmaceutical composition of embodiment 31, wherein the first virus comprises a first nucleic acid sequence encoding one or more anti-cancer protein and the second virus comprises a second nucleic acid sequence encoding one or more anti-cancer proteins.

Embodiment 33. The pharmaceutical composition of embodiment 31 or 32, further comprising a third virus.

Embodiment 34. The pharmaceutical composition of embodiment 33, wherein the third virus comprises a third nucleic acid sequence encoding one or more anti-cancer proteins.

Embodiment 35. A cell comprising the virus of any one of embodiments 1-26.

Embodiment 36. A method of treating or preventing cancer in a subject in need thereof, said method comprising administering to said subject a therapeutically or prophylactically effective amount of the virus of any one of embodiments 1-26.

Embodiment 37. The method of embodiment 36, wherein said cancer is a gynecological cancer.

Embodiment 38. The method of embodiment 36 or 37, wherein said cancer is ovarian cancer, uterine cancer, or cervical cancer.

Embodiment 39. The method of any one of embodiments 36-38, wherein said cancer is ovarian cancer.

Embodiment 40. The method of any one of embodiments 36-39, wherein said cancer expresses CA-125.

Embodiment 41. A method of stimulating an immune response in a subject in need thereof, said method comprising administering to said subject an effective amount of the virus of any one of embodiments 1-26.

Embodiment 42. The method of embodiment 41, wherein the subject has or previously had a CA-125 expressing cancer.

Embodiment 43. The method of any one of embodiments 36-42, wherein said virus is administered at a dose from about 10¹⁰ to about 10¹² plaque forming units (Pfu).

Embodiment 44. The method of any one of embodiments 36-43, wherein said virus is administered intraperitoneally, intratumorally, intravenously, intrathecally, or intrapleurally.

Embodiment 45. The method of embodiment 44, wherein said virus is administered intraperitoneally.

Embodiment 46. A method of treating or preventing cancer in a subject in need thereof, said method comprising administering to said subject a therapeutically or prophylactically effective amount of a cell comprising the virus of any one of embodiments 1-26.

Embodiment 47. The method of embodiment 46, wherein said cancer is a gynecological cancer.

Embodiment 48. The method of embodiment 46 or 47, wherein said cancer is a CA-125 expressing cancer.

Embodiment 49. The method of any one of embodiments 46-48, wherein said cell is a CA-125 expressing cancer cell.

Embodiment 50. The method of any one of embodiments 46-49, wherein said cell is administered intraperitoneally, intratumorally, intravenously, intrathecally, or intrapleurally.

Embodiment 51. The method of embodiment 50, wherein said cell is administered intraperitoneally.

Embodiment 52. A method of stimulating an immune response in a subject in need thereof, said method comprising administering to said subject an effective amount of a cell comprising the virus of any one of embodiments 1-26.

Embodiment 53. The method of embodiment 52, wherein said subject has or previously had a CA-125 expressing cancer.

Embodiment 54. The method of embodiment 52 or 53, wherein said cell is a CA-125 expressing cancer cell.

Embodiment 55. A method of inhibiting proliferation of a CA-125 expressing cell, said method comprising contacting said CA-125 expressing cell with the virus of any one of embodiments 1-27.

Embodiment 56. The method of embodiment 55, wherein said CA-125 expressing cell is in a subject having cancer.

Embodiment 57. The method of embodiment 56, wherein said cancer is a gynecological cancer.

Embodiment 58. The method of embodiment 56 or 57, wherein said cancer is ovarian cancer, uterine cancer, or cervical cancer.

Embodiment 59. The method of any one of embodiments 55-58, wherein said CA-125 expressing cell is a CA-125 expressing cancer cell.

### EXAMPLES

### Example 1: Background to Exemplary Experiments

Ovarian cancer, referred to as ovarian epithelial, fallopian tube, or primary peritoneal cancer (1), is the fifth most common cause of cancer-related death of women in the US, with an estimated 21,410 new cases and 13,770 deaths in 2021 (American Cancer Society, Cancer Facts & Figures, 2021). Due to lack of an effective screening test (2), ovarian cancer typically presents at a later stage (FIGO stage III or IV), where one third of patients have malignant ascites at initial presentation (3). Current treatment of ovarian cancer is primarily limited to surgery and chemotherapy, and the five-year survival rate in the US is below 50% (SEER Cancer Statistics Review). New treatments are urgently needed to help patients suffering from this deadly disease.

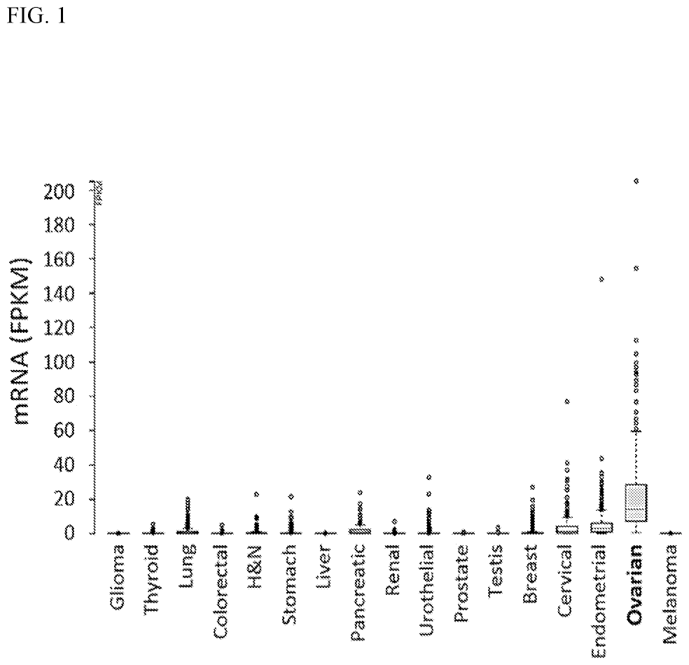

A unique feature of ovarian cancer is that more than 80% of patients express a high serum level of CA-125, one of the largest, hyperglycosylated, human proteins, expressed in and shed from ovarian cancer cells. CA-125 levels in ovarian cancer patients can reach up to hundreds and thousands of units per milliliter (U/ml), while most other cancer patients and individuals without cancer have a low level of CA-125, below 35 U/ml or undetectable (2, 4, 5). Bioinformatic analysis shows that CA-125 mRNA is also highly expressed in ovarian cancer cells (ACGT database, FIG. 1), but not in most other cancer cells or in normal cells.

The gene coding for CA-125, termed MUC16, is located on chromosome 19p13.2 and is comprised of approximately 179 kb of genomic DNA (7, 15). It remains unknown why CA-125 is highly expressed in gynecological cancer cells, especially in ovarian cancer cells and not in other cancer cells or normal cells. Specific transcriptional activation of MUC16 gene in ovarian cancer cells is poorly defined (15, 16). Nevertheless, the goal is to determine if it is feasible and practical to target the specific MUC16 gene transactivation by developing a conditionally replicative adenovirus (CRAd) that can only replicate in ovarian cancer cells expressing CA-125 and if the virus-infected cancer cells can induce a protective anti-cancer immune response for ovarian cancer treatment.

To Applicant's knowledge, targeting specific MUC16 transactivation in ovarian cancer for treatment has never been attempted. Results described here demonstrate that this strategy of constructing a CRAd dependent on MUC16 transactivation for replication is feasible and practical for ovarian cancer treatment and provide a potent targeted agent that not only can specifically replicate in and lyse ovarian cancer cells, but also can effectively activate a protective anti-cancer immune response. Successful identification of the MUC16 transcriptional elements also provides an avenue for future mechanistic investigations into CA-125 regulation in ovarian cancers, which may reveal a new field to further explore ovarian cancer treatment.

Applicant obtained a 5 kb upstream DNA fragment (SEQ ID NO:3) of MUC16 by multiple steps of high-fidelity PCR on genomic DNA isolated from the OVCAR3 ovarian cancer cell line and cloned it into a firefly luciferase reporter vector pGL4.14. Applicant showed it processes specific transactional activity in CA-125-high expressing cancer cells with minimum activity in CA-125-low cancer cells. Described herein is construction of conditionally replicative oncolytic viruses with the MUC16 transactivation sequence to control an essential gene for virus replication is an innovative. Applicant showed that this is a practical way to target the specific MUC16 transactivation for ovarian cancer treatment.

Studies described herein include 1) Development of a potent CRAd that specifically replicates in ovarian cancer cells expressing CA-125. Applicant identified and refined the transcription activation region of MUC16 to facilitate construction of a CRAd with high oncolytic activity and immunogenicity. 2) Evaluation of the anti-cancer activity of the CRAd in immunodeficient and immunocompetent mice. Applicant accessed the ability of the CRAd to replicate in and destroy ovarian cancer cells expressing CA-125 using human ovarian cancer xenograft mouse models in immunodeficient mice and immunogenicity of virus-infected mouse ovarian cancer cells in immunocompetent mouse models. 3) Analysis of the oncolytic activity of the CRAd in primary ovarian cancer cells collected from patients, in preparation for a phase I clinical trial of the oncolytic virus in ovarian cancer patients.

### Example 2: Oncolytic Viruses for Targeting MUC16 Expressing Cancer Cells

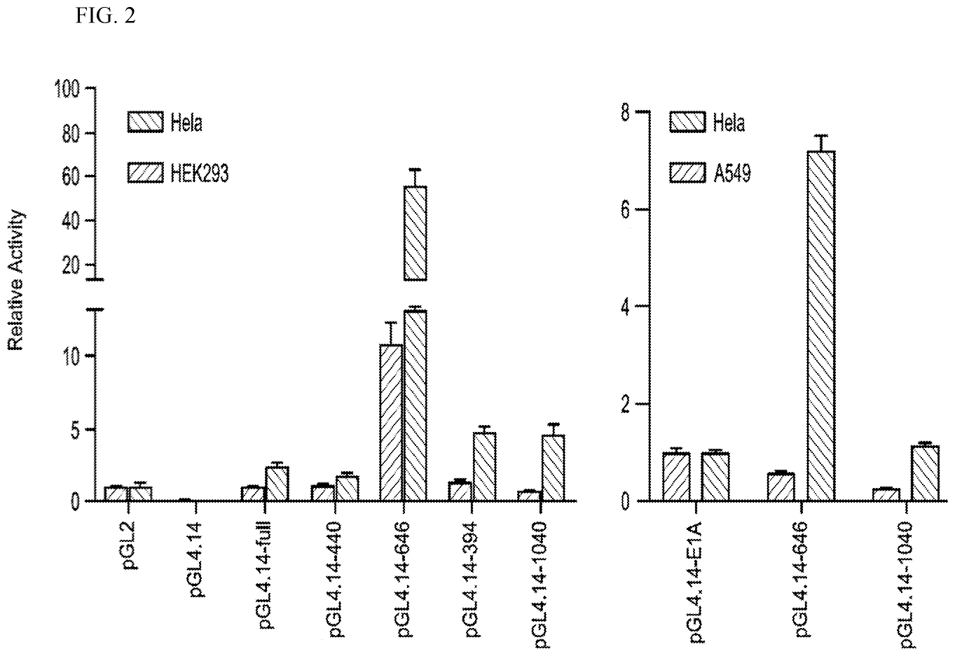

Applicant retrieved the DNA sequence of Homo sapiens mucin 16 in chromosome 19p13.2 (NG_055257) from the human genome database. Applicant then performed multiple steps of high-fidelity PCR on genomic DNA isolated from the OVCAR3 ovarian cancer cell line and eventually obtained a 5 kb MUC16 gene fragment, which contains a region upstream from the transcription start site (TSS) and a 0.4 kb region downstream from the TSS, along with both the 5′ untranslated region (5′UTR) and the first 136 bp of the open reading frame (ORF). Applicant cloned this DNA fragment into the multi-cloning site of a firefly luciferase reporter vector pGL4.14 (Promega). HEK292 cells and Hela cells were chosen to test firefly luciferase activity, because CA-125 mRNA is low (0 NX) in HEK293 cells and high (26.9 NX) in Hela cells, per the Human Protein Atlas (https://www.proteinatlas.org/ENSGOOOOO181143-MUC16/cell). As shown in FIG. 2, left panel, pGL4.14-MUC16-full induced higher expression of firefly luciferase activity in Hela cells, but not in HEK293 cells, compared to the pGL-2 reporter carrying a firefly luciferase gene under control of the SV40 large T promoter, a promoter used in many eukaryotic expression vectors to promote transcription in most mammalian cells. Applicant further refined the promoter and found that a fragment of 1040-bp, including a CT-rich region, TATA box, TSS, showed a high transactivation activity in Hela cells, 7 times more active than that in HEK293 cells (FIG. 2, left panel).

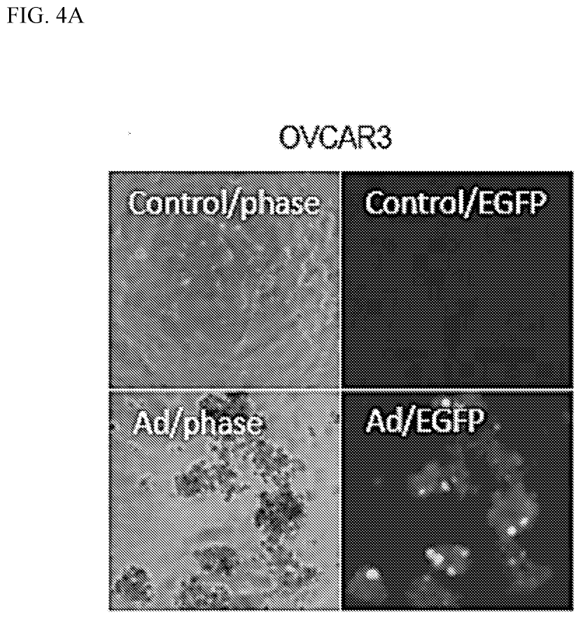

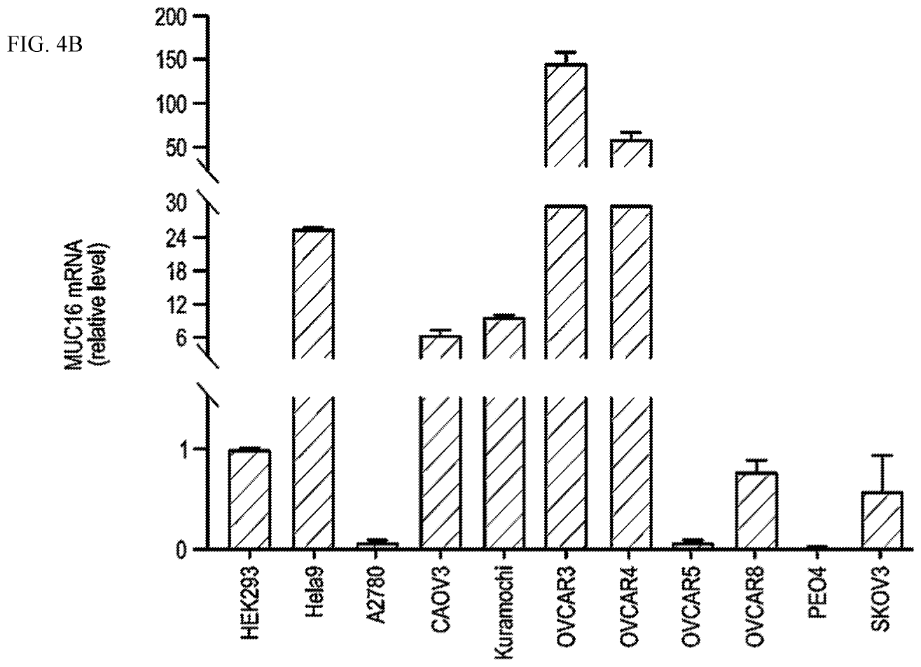

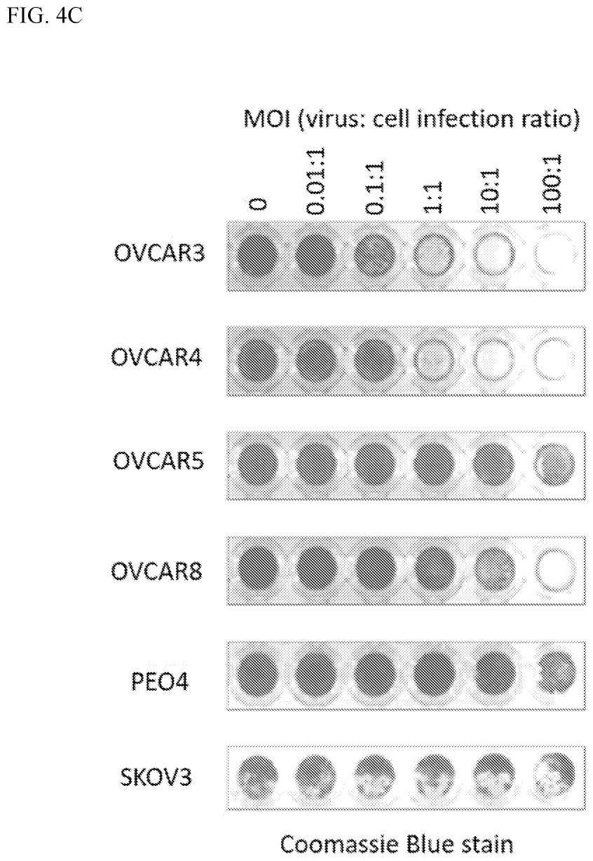

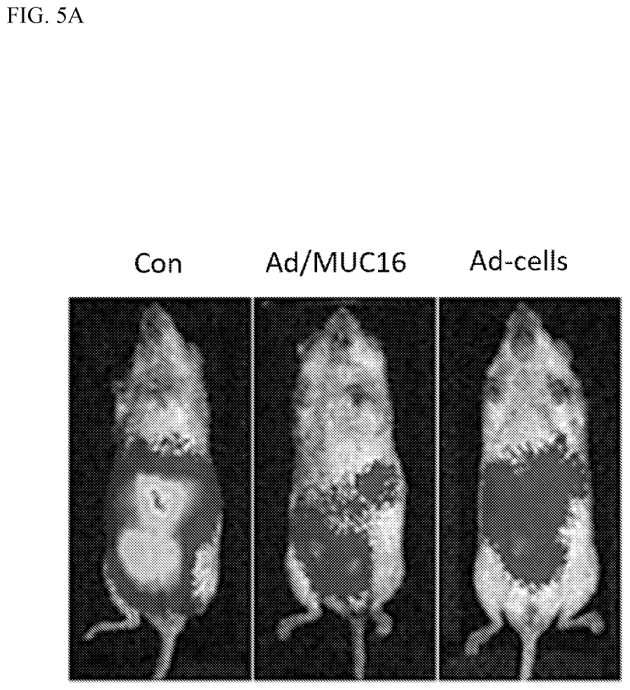

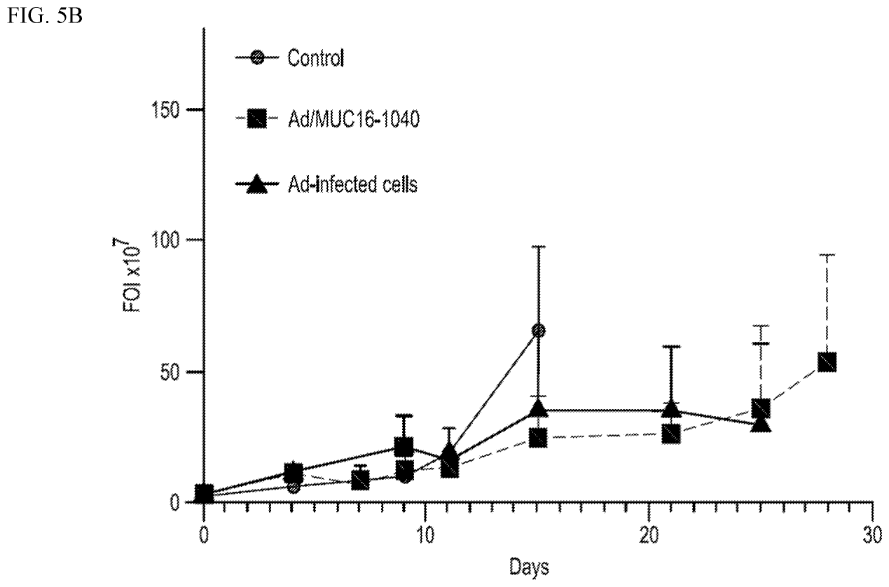

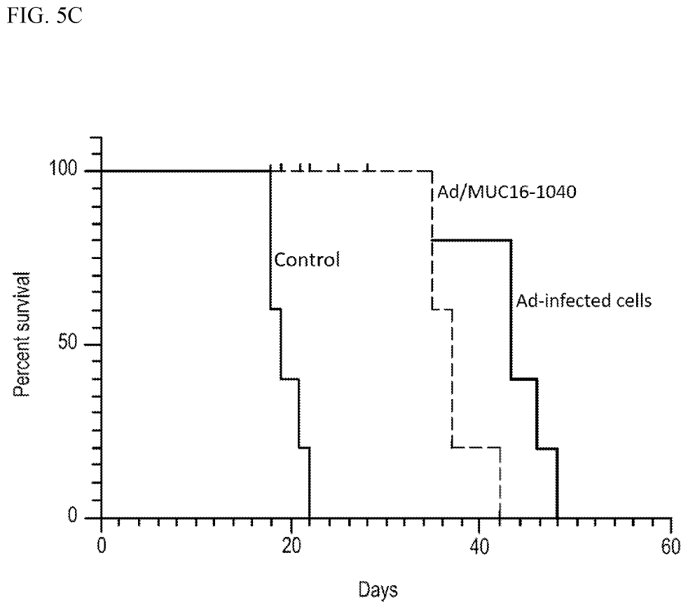

Applicant next compared the MUC16-1040 fragment with the Ad5/E1A promoter. As shown in FIG. 2, right panel, the MUC16-1040 fragment showed equivalent or slightly higher activity in HeLa cells compared to the E1A promoter, but much lower activity (27.3%) in the A549 lung adenocarcinoma cell line, which is characterized by ease of use for adenovirus amplification and low expression of CA-125 (FIG. 4B). Identification of a MUC16 fragment around 1 kb or less with promoter activity that specifically controls CA-125 expression made it possible to engineer the adenovirus type 5 for targeted cancer therapy.

Generation of a CRAd with MUC16 Fragment with Promoter Activity to Control E1A Expression.

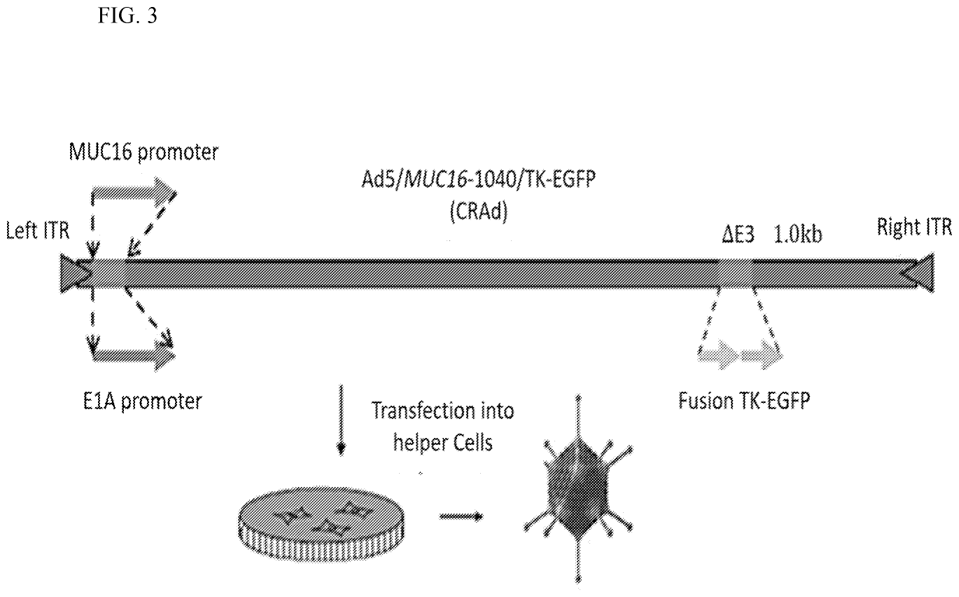

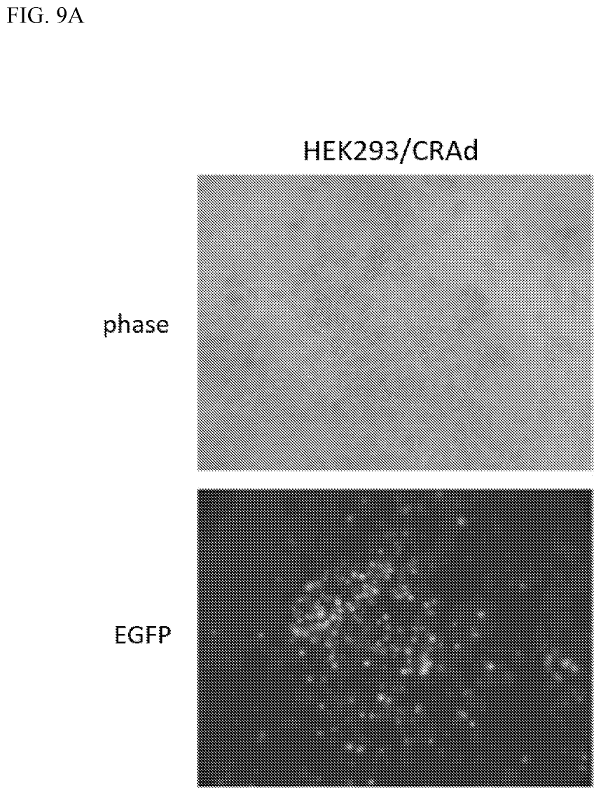

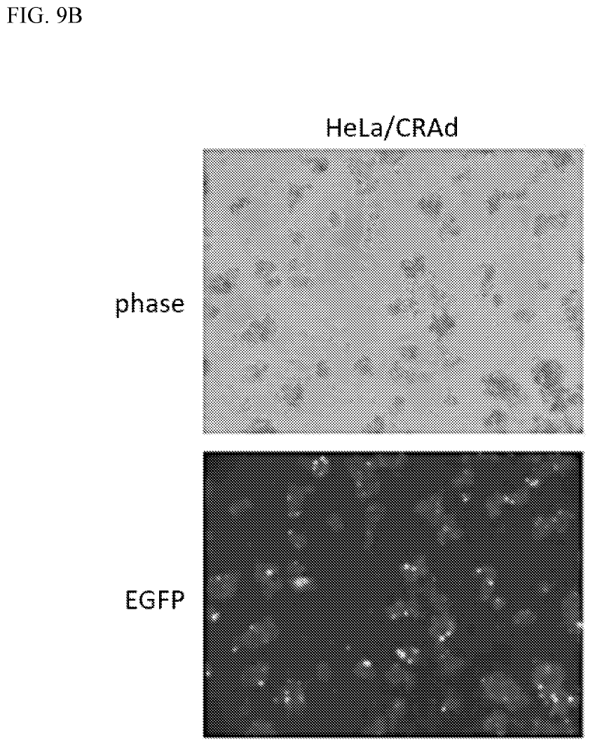

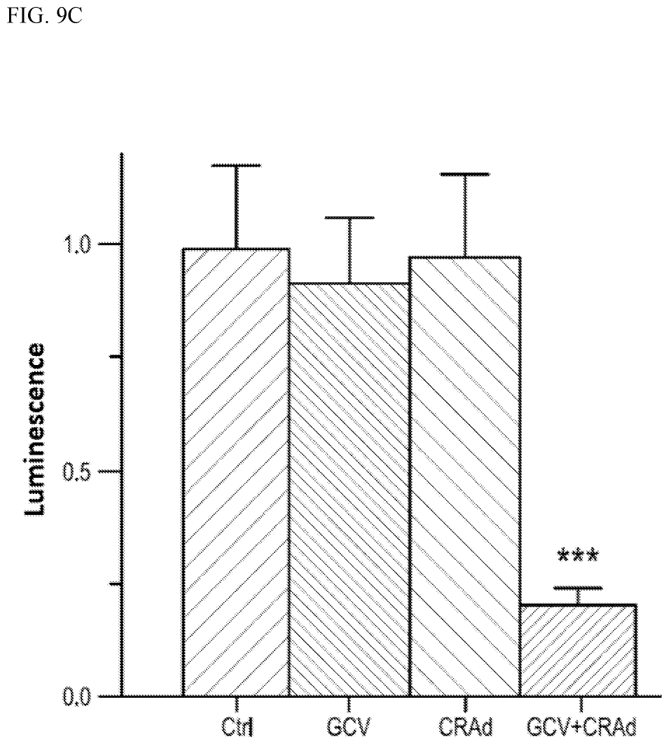

Applicant identified and refined the transcription activation region of MUC16 to facilitate construction of a CRAd with high oncolytic activity and immunogenicity. Adenovirus construction was initiated using this 1040-bp fragment (SEQ ID NO:1) and made the first version of conditionally replicative adenovirus. The AdenoQuick2.0 system was used (OD260 Inc.), which consists of four shuttle vectors (pAd1127, pAd1129, pAd1128, and pAd1130) with all the components needed for recombined adenovirus construction. pAd1127 contains the left end of the wild-type adenovirus type 5 genome with an E1A expression cassette. Applicant started virus construction with the MUC16-1040 bp promoter replacing the E1A promoter in pAd1127, where E1A is an essential gene for adenovirus replication (29-31). The TSS of the MUC16 promoter was kept and fused with the TSS of E1A for proper expression. At the same time, the pAd1129 shuttle vector was constructed to express TK-eGFP at the E3 region. TK-eGFP is a fusion protein with both active eGFP that can be used to track virus infection and herpes simplex virus-1 thymidine kinase (HSV-TK) activity that can convert ganciclovir (GCV) or analogues into a toxic metabolite to kill cells (32). The HSV-TK gene was used for two reasons. First, ganciclovir can be delivered to kill host cells, if viral replication is limited and insufficient to destroy cells. Second, if viral replication is out of control, pro-drug can be used to kill the host cells before mature viruses are generated and infect other cells, thereby limiting cytotoxic effects. All adenovirus genome fragments were collected from the 4 vectors and ligated to generate the full adenovirus genomic DNA, which was cloned to a phage X vector for amplification in E. Coli. Finally, the recombined adenovirus genome DNA was excised and transfected it into HEK293 cells, a human embryonic kidney cell line immortalized by Ad5 E1A/B that is routinely used for adenovirus packaging and amplification, because it expresses abundant E1A protein in the cells(33). In these HEK293 cells, a recombined adenovirus was successfully generated and termed Ad5/MUC16-1040/TK-eGFP, as illustrated in FIG. 3. This virus induced classic cyto-pathic effects (CPE) in CA-125-high cells, including HeLa (FIG. 9B) and OVCAR3 (FIG. 4A). Applicant further tested sensitivity of the virus-infected cells to GCV. As shown in FIG. 9C, mouse ovarian cancer cell line ID8 became more sensitive to GCV when infected with the virus.

Replication and Oncolysis of Ad5/MUC16-1040 in Ovarian Cancer Cell Lines is Dependent on CA-125 Expression

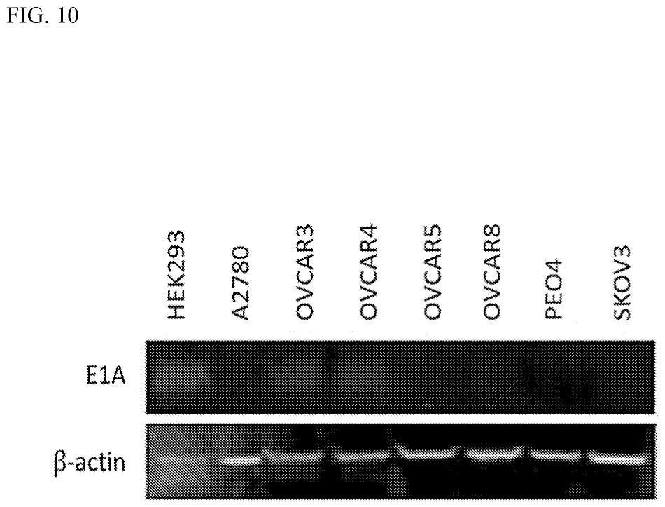

Applicant tested Ad5/MUC16-1040/TK-EGFP in a panel of cell lines, including HeLa, HEK293, A2780, A549, BEAS-2B, CAOV3, HeyA8, Kuramochi, OVFB-1, OVCAR3, OVCAR4, OVCAR5, OVCAR8, PEO4, and SKOV3 for replication and oncolysis to determine if it is correlated with CA-125 expression in these cells. The CA-125 mRNA level in HEK293 cells is low [21], so it served as a comparator for the mRNA levels in other cell lines. As shown in FIG. 4B, HeLa cells expressed a relatively high level of CA-125, 26.1 times higher than HEK293 cells, which is consistent with the Protein Atlas database [21]. OVCAR3, OVCAR4, CAOV3, and Kuramochi expressed a high level of CA-125 mRNA, with OVCAR3 having the highest, 145.8 times higher than HEK293 cells. Other cells, including A2780, OVCAR5, OVCAR8, PEO4, and SKOV3, expressed low levels of CA-125 mRNA. Applicant found that Ad5/MUC16-1040/TK-eGFP replicated in and lysed CA-125 high-expressing cancer cells, including Hela, OVCAR3 (FIG. 4A, 4B, 4C), OVCAR4 (FIG. 4B, 4C), CAOV3, MADH2774, and Kuramuchi cancer cells. Furthermore, amplified viruses released from the cancer cells upon cell lysis can infect and kill other cells. The virus did not replicate well in CA-125 low or negative cancer cell lines, A2780, OVCAR5, or PEO4 (FIG. 4B, 4C). Thus, Applicant determined that replication and amplification capacity of the virus correlates with CA-125 mRNA levels in the ovarian cancer cells (FIG. 4B, 4C, and data not shown). Amplified viruses released from the cancer cells upon cell lysis can subsequently infect and kill other cells. The virus does not replicate well in the CA-125 low or negative cancer cell lines A2780, OVCAR5, SKOV3, or PEO4 (FIG. 4B, 4C). Thus, it appears that oncolysis mediated by the virus correlates to CA-125 mRNA expression in this panel of cells. Of importance, the ovarian cancer cells lines, A2780 [25,26], OVCAR5 [19], and PEO4 [27] express coxsackievirus and adenovirus receptor (CAR) on their cell surface, and they are permissive of wild-type adenovirus replication; however, these cells are resistant to the virus, and no obvious CPE were observed even when infected at an MOI of 100:1. Both OVCAR3 and SKOV3 have limited CAR expression, but it is sufficient for wild-type adenovirus to infect and replicate in these two cell lines [28]. The virus can infect, replicate, and cause CPE in OVCAR3 cells, but not in SKOV3 cells, which Applicant believe is due to high expression of CA-125 in OVCAR3 cells and low expression in SKOV3. Applicant also determined E1A expression levels via Western blot in selected ovarian cancer cell lines infected with Ad5/MUC16-1040/TK-EGFP. As shown in FIG. 10, E1A expression is high in the virus-infected OVCAR3 and OVCAR4, but low or undetectable in A2780, OVCAR5, OVCAR8, PEO4 and SKOV3.

Further, Applicant collected normal cell lines, including the HFF human foreskin fibroblasts, the human lung epithelial cell line BEAS-2B, and two human fibroblast cell lines derived from ovarian tumor tissue established in the lab, and tested MUC16 promoter containing virus on them. As expected, Ad5/MUC16-1040/TK-EGFP does not induce apparent CPE in these normal cells at an MOI up to 100:1 after a period of 2 weeks in culture with regular passaging every 37 days, although EGFP signal could be seen 3-5 days after infection, which gradually disappeared with continued cell passaging (data not shown). Collectively, these results indicate that MUC16 promoter containing virus can replicate in and lyse cancer cells expressing CA-125, not in cancer cell without CA-125 expression or in normal cell lines.

Of interest, Applicant found that Ad5/MUC16-1040/TK-eGFP can also infect and replicate in mouse ovarian cancer cell lines, ID8 and STOSE, although to a lesser degree. This made it possible to test whether virus infection of mouse ovarian cancer cells can elicit protective immune responses in immunocompetent mice when re-challenged with parental cancer cells. To achieve a robust anticancer immune response, a new Ad5/MUC16-1040 virus was generated that carry an expression cassette of mouse GM-CSF in the E3 region of the adenovirus genome, now termed as Ad5/MUC16-1040/mGM-CSF. With this modification, it was thought that cancer cells infected with the virus can elicit an effective antitumor immune response to eradicate uninfected cells.

Further the virus was engineered to carry different cytokines, such as IL-2, IL-12, costimulatory signals, B7-1, CD137L (4-1BBL) to test their anti-cancer immunity-inducing capacity. Studies with varying cytokines (e.g. immune stimulatory molecules) are described in more detail below.

A second-generation of CRAd was constructed by a further refined 646-bp DNA fragment (SEQ ID NO:2) that demonstrated high transcription activity, 6-7 times higher than the 1040-bp fragment (SEQ ID NO:1), along with the anticipated differential transactivation in Hela cells versus HEK293 cells (FIG. 2, left, 3^{rd} set of bars from right). Applicant also compared the 646-bp promoter with the adenovirus E1A promoter in terms of transcriptional activity driving a firefly luciferase gene in the pGL4.14 vector. As shown in FIG. 2 (right), transcriptional activity of the 646-bp MUC16 promoter is 12 times higher than the wild-type adenovirus E1A promoter in Hela cells. However, in A549 cells, a cell line commonly used for replication competent adenovirus amplification, there is limited activity, only 59% that of the E1A promoter. By constructing the second-generation virus using the 646-bp promoter to control E1A expression, it was thought that Ad5/MUC16-646/TK-eGFP demonstrates significantly increased oncolytic activity and has a favorable toxicity profile relative to the wild-type adenovirus.

The promoter was further refined and the virus is modified to generate a panel of viruses with specific oncolytic activity and immune regulatory activity. Anti-cancer efficacy was evaluated in animal models to identify the CRAd with highest anti-cancer potency.

Oncolytic Activity in Ovarian Cancer Xenograft Models.

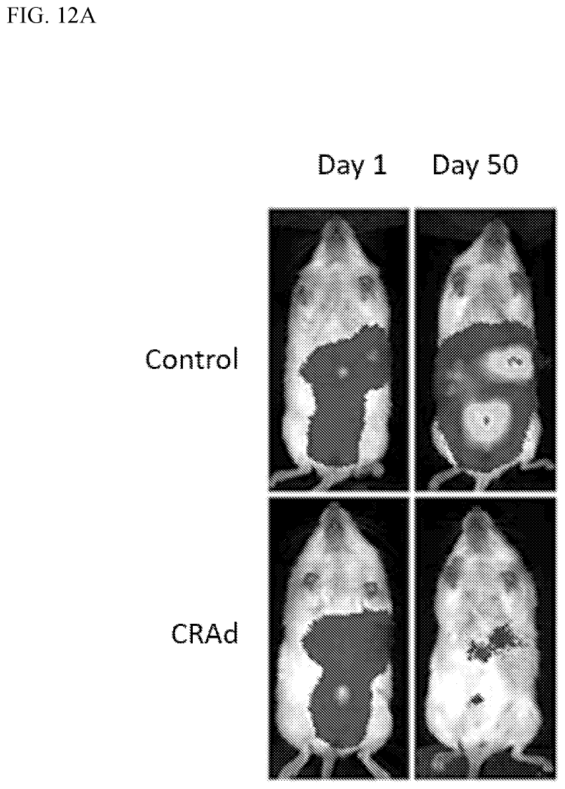

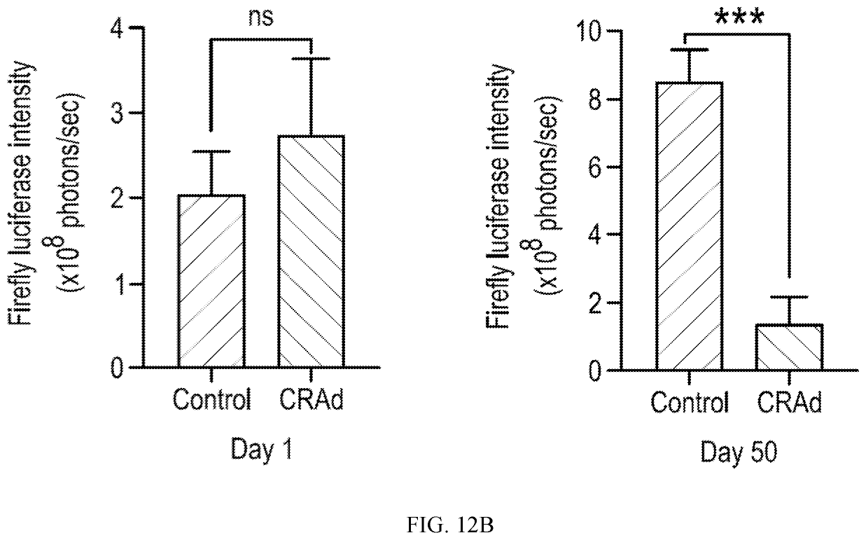

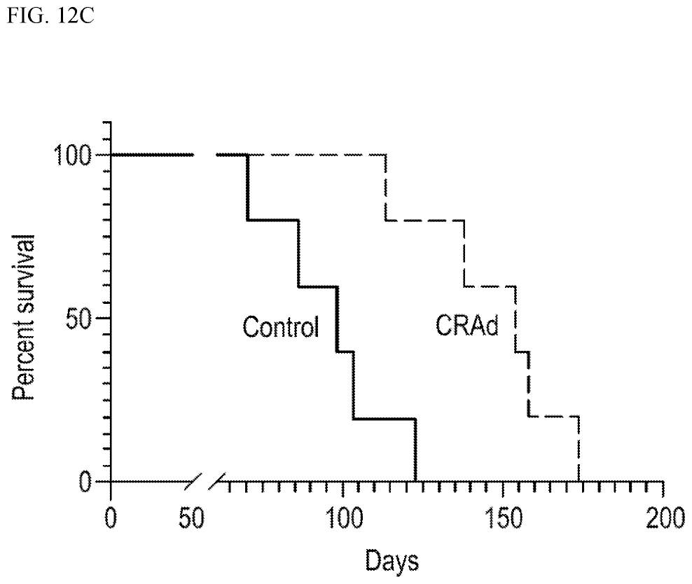

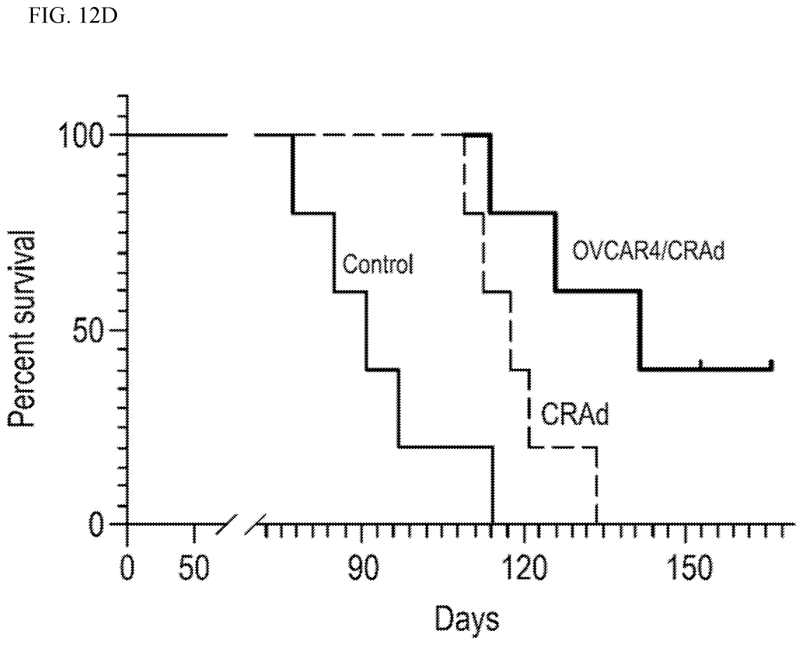

To assess oncolytic activity in vivo, Applicant tested Ad5/MUC16-1040/TK-EGFP in an ovarian cancer xenograft mouse model using NOD-scid IL2Rgamma-null (NSG) immunodeficient mice. For these in vivo studies, CA-125-high-expressing Kuramochi human ovarian cancer cells labeled with a firefly luciferase gene to track tumor growth were selected. As the peritoneum is the primary site of ovarian cancer progression, cancer cells were inoculated into peritoneum of NSG mice on day 0 to establish tumor xenografts. Then virus was directly injected into the peritoneum of the tumor-bearing mice on day 1, up to 1×10¹⁰⁻¹² plaque forming unit (pfu) per mouse, and tumor growth was monitored with the Lago X Spectral Instruments Imaging System. As shown in FIGS. 12A and 12B, firefly luciferase intensity increases over time in the control group; however, the intensity of firefly luciferase in mice treated with ip injection of Ad5/MUC16-1040/TK-EGFP was significantly lower than the control group on day 50. NSG mice treated with ip injection of Ad5/MUC16-1040/TK-EGFP virus survived longer than control group (FIG. 12C). OVCAR4 cells were also inoculated ip on day 0, and the Ad5/MUC16-1040/TK-EGFP virus was injected ip on day 8. As shown in FIG. 12D, NSG mice treated with intraperitoneal injection of Ad5/MUC16-1040/TK-EGFP virus survived longer than control group.

As the adenovirus particle is extremely small (diameter of about 80-120 nm) and the peritoneum cavity is large, especially in humans, virus injected directly to peritoneum may diffuse rapidly into the body or may be inactivated after injection therefore limit infection efficiency to cancer. cells. Thus, virus-infected cells were also injected, which carried and amplified the virus in the peritoneum and released virus upon maturation to infect other cancer cells. Intraperitoneal injection of ovarian cancer cells is practical clinically, as they can be easily obtained from ascites. Data obtained using this approach were positive, as shown in FIGS. 12A-12D. As shown in FIG. 12D, intraperitoneal delivery of virus by Ad5/MUC16-1040/TK-EGFP-in-fected OVCAR4 cells used as a carrier also suppressed tumor cell growth and prolonged mouse survival. At the time of preparing this submission, corresponding to 166 days post-injection, two mice in the cohort remained alive without any evidence of cancer growth. Of note, MDAH2774 is a rapid growing cell line, and adenovirus spread is usually restricted by the microenvironment (21, 22), thus it was not a surprise that the virus was unable to eradicate all the cancer cells in vivo.

Applicant optimized virus dose and delivery frequency, and monitored how well tumor xenograft progression was controlled with the virus in the xenograft model. The virus was tested on other cell lines, such as OVCAR3 and OVCAR4, which express high levels of CA-125 (FIG. 4B) and grow slowly, which allowed optimal replication of virus and facilitate improved responses to virus infection. Furthermore, Applicant constructed a new adenovirus with a stronger promoter, MUC16-646 bp promoter, which can yield more potent oncolytic activity in animal models.

Another approach is intratumoral injection of the virus in human ovarian cancer xenografts in NSG mice. Applicant inoculates OVCAR3/FL or OVCAR4/FL cells subcutaneously into both flanks of the mouse. When the tumor mass grows to about 100-200 mm³, Applicant injects the virus with luciferase activity with the Lago X Spectral Instruments Imaging System; the tumor on the other side serves as a control. It is thought that the virus can infect at least part of the tumor cells and replicate in the cells to slow down tumor growth or eradicate the tumor entirely.

Anti-cancer efficacy is evaluated in a human patient-derived xenograft (PDX) model of ovarian cancer. With IRB- and IACUC-approval, Applicant is collecting ovarian cancer tissues from patients with high CA-125 levels to establish these PDX models. Ovarian cancer PDX models are also commercially available from sources such as Jackson Laboratories and Anticancer Inc, both sources provide information on CA-125 expression levels. Tumor tissues are inoculated subcutaneously into NSG mice and tumor growth monitored to a volume of about 300 mm³. Applicant then injects into each tumor mass at 1×10¹⁰ pfu and measures and records the tumor size regularly to monitor tumor growth.

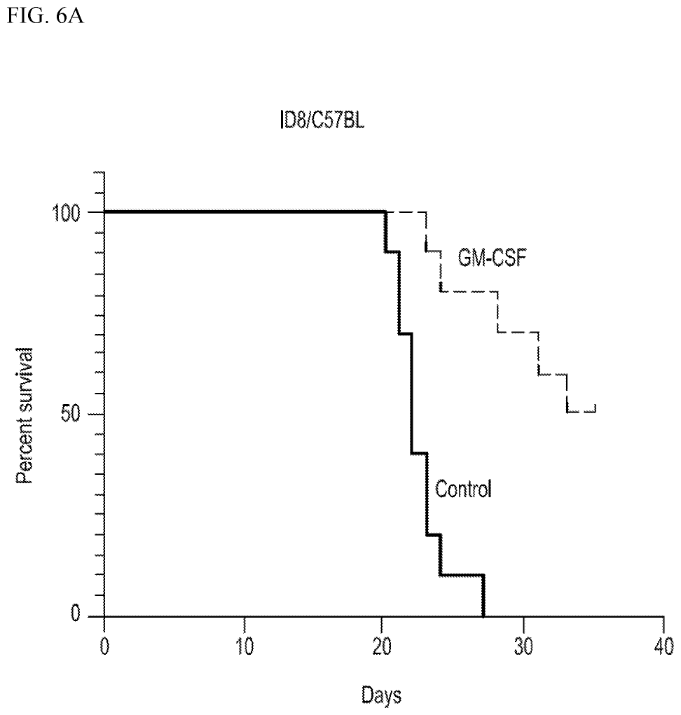

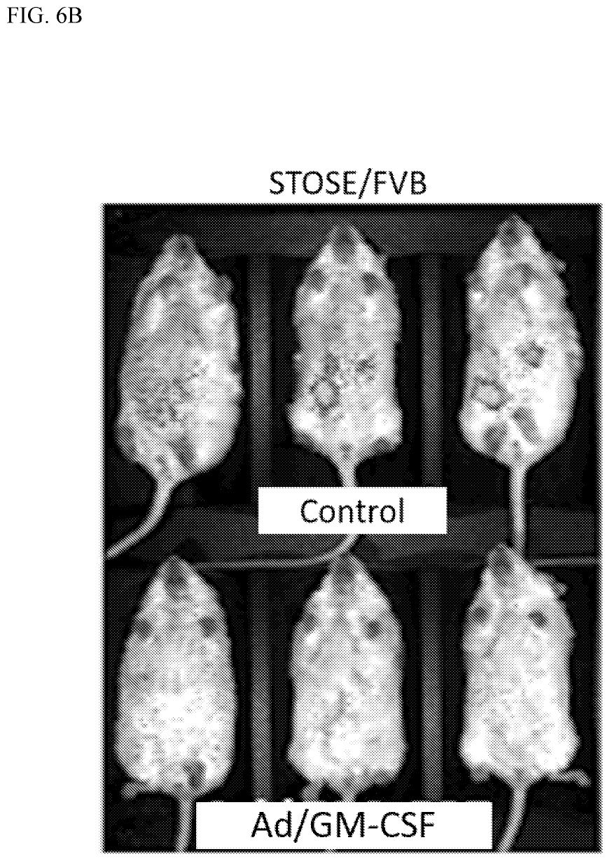

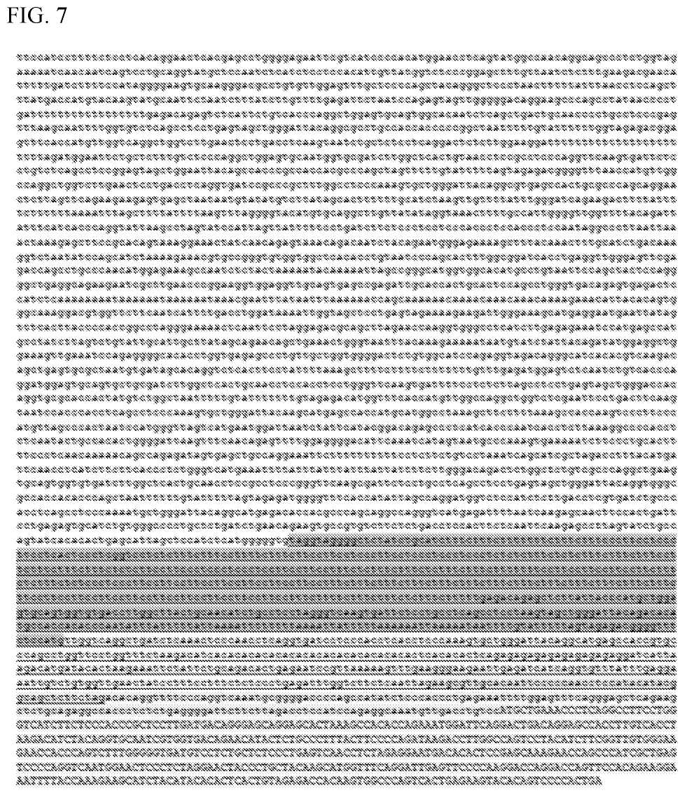

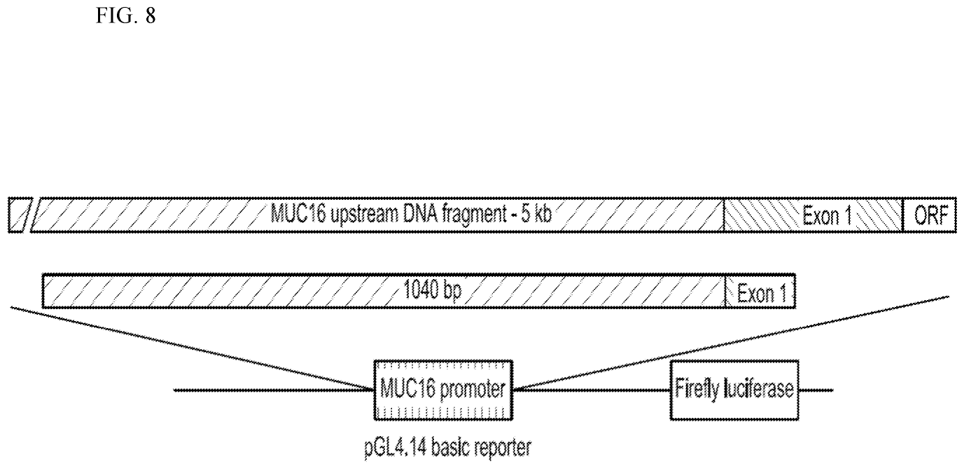

Applicant assessed anti-cancer immune responses in immunocompetent mouse models of ovarian cancer. Investigation of whether virus infection of mouse ovarian cancer cell lines can activate a protective immune response was tested in immunocompetent mice subjected to re-challenge with parental cancer cells. Data indicate that inoculation with virus-infected STOSE cancer cells can elicit a protective effect against re-challenge with parental STOSE cancer cells in FVB/NJ mice, while virus infected ID8 cancer cells do not induce any protective effect in C57BL/6 mice. These findings were not surprising, because mouse ID8 is a poorly immunogenic ovarian cancer cell line that rarely induces a protective immune response to ID8 re-challenge, per the literature (26, 27). However, incorporation of GM-CSF into the virus changed the outcome. Subcutaneous pre-inoculation of ID8 cells infected with Ad5/MUC16-1040/mGM-CSF significantly prolonged survival of C57BL/6 mice re-challenged intraperitoneally with parental ID8 cells (FIG. 6A). The protective effect is more robust in STOSE cancer cells in FVB/NJ mice (FIG. 6B, 3 representative mice from each group), where the mice harbor no significant residual disease thus far. More viruses that carry other key immunoregulatory genes are constructed, individually or in combination, and their anti-cancer immune-inducing capacity is evaluated using these syngeneic ID8/C57BL/6 and STOSE/FVB/NJ mouse models of ovarian cancer.

Selective Replication and Oncolysis in Primary Ovarian Cancer Cells.

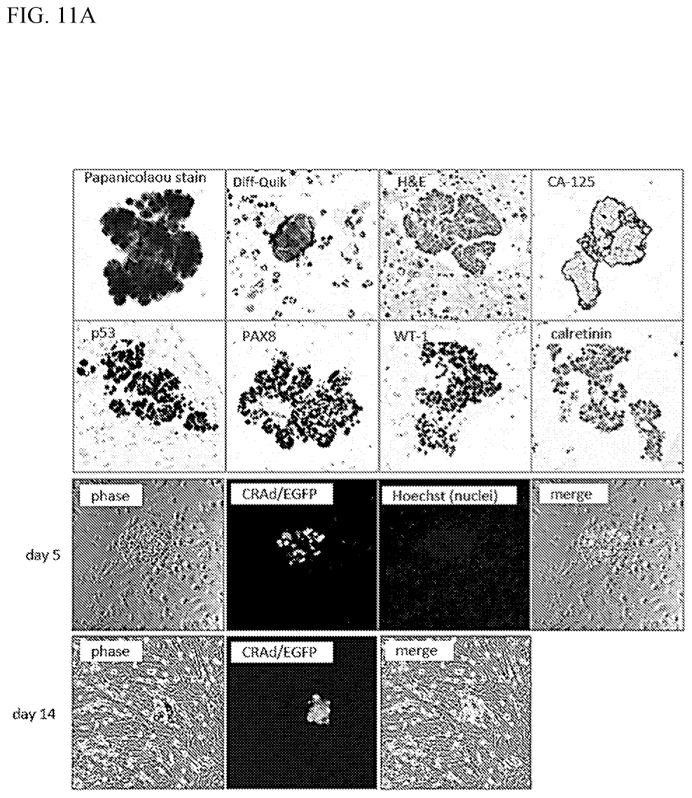

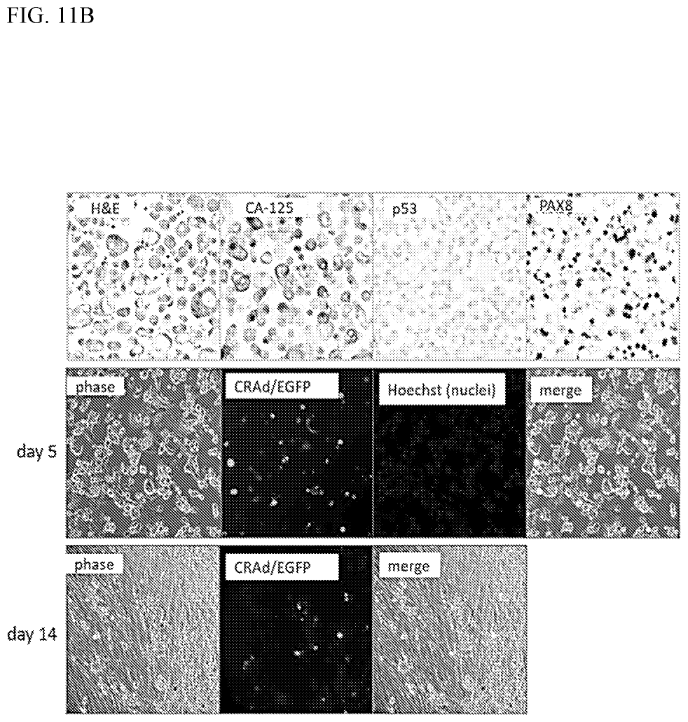

For translational purposes, applicant tested the Ad5/MUC16-1040/TK-EGFP on primary ovarian cancer cells collected from two patients; case 1 was from pleural effusion, and case 2 was from ascites. Cytology confirmed the presence of malignant cells positive for CA-125 in both cases, as shown in FIG. 11A (case 1) and FIG. 11B (case 2). Immunohistochemistry showed that the malignant cells were positive for PAX8 (FIG. 11A, 11B) and WT1 (FIG. 11A, and data not shown), negative for mesothelial marker, calretinin (FIG. 11A, and data not shown), p53 mutation pattern (FIG. 11A, abnormal strong diffuse expression), and p53 null pattern (FIG. 11B), consistent with a high grade serous carcinoma of ovarian primary origin of both cases.

Applicant infected the collected cells with Ad5/MUC16-1040/TK-EGFP. Five days after infection, EGFP signals were seen in cancer cells, which was easy to recognize when clustered together (FIG. 11A, case 1, day 5) under an inverted phase contrast microscope. A few days later, the EGFP positive cells showed classic CPE, floated and died eventually as shown in FIG. 11A (day 14). Surrounding cells, mostly fibroblasts, continued to grow and replaced the culture surface where cancer cells had been attaching. Case 2 has a large number of malignant cells that are sensitive to the virus infection indicated by EGFP signal as shown in FIG. 11B (case 2, middle panel, day 5), with limited normal cells in culture. A few days later, cancer cells floated and disappeared gradually, while fibroblasts took place of the culture surface (FIG. 111B, lower panel, day 14). The data indicates that Ad5/MUC16-1040/TK-EGFP indeed can replicate in and lyse primary cancer cells collected from patients, but not in normal cells.

### DISCUSSION

Transcriptional regulation of MUC16 in ovarian cancer cells has been poorly defined (16, 17), and targeting specific transactivation of MUC16 in ovarian cancer cells has not been exploited for cancer treatment. Success in developing a CRAd that can only replicate in and lyse cancer cells expressing CA-125 provides proof-of-concept that transactivation of MUC16/CA-125 can be targeted for ovarian cancer treatment.

Applicant obtained a 5 kb DNA fragment (SEQ ID NO:3) upstream of the human MUC16 gene cloned to a firefly luciferase reporter vector and found that it processes differential transcriptional activity in CA-125-expressing cells versus non-expressing cells (FIG. 2 left, 3rd set of bars from left). Applicant further refined to a 1040-bp promoter (SEQ ID NO:1) that reserved the differential transcriptional activity (FIG. 2 left, the 7th set of bars from left). Transfection of toxic gene, Herpes Simplex Virus type 1 (HSV-1) thymidine kinase (TK), driven by this promoter in an eukaryotic expression vector induced cell death in ovarian cancer cells with CA-125 expression in the presence of ganciclovir. However, gene delivery in animal models and in the clinic so far is not practical for therapeutic purposes. The method of constructing a conditionally replicative oncolytic virus with the MUC16 transactivation sequence to control virus replication is an ideal and innovative approach for ovarian cancer treatment. Using this MUC16-1040 bp fragment to replace the adenovirus (Ad5) E1A promoter to control expression of E1A, an essential gene for adenovirus replication, Applicant successfully generated a CRAd that can only replicate in and lyse cancer cells with high CA-125 expression, but not cells with low or no CA-125 expression.

Among all the oncolytic viruses, Applicant chose adenovirus as the platform for testing for the following reasons: First, adenovirus is well characterized, demonstrating a relatively safe toxicity profile and a broad spectrum of targeted cells, including cancer cells and normal cells. Second, it is a nonenveloped virus with an icosahedral nucleocapsid containing a double stranded DNA genome. Recently, adenovirus has served as an ideal vector for vaccine development [30], including many of the COVID-19 vaccines [31]. Third, human adenovirus can cross species, infect and replicate in mouse cancer cells [32], although to a lesser degree, a feature allows us to investigate oncolytic virus-induced immune responses in immune-competent mice. Thus, adenovirus is highly immunogenic and can induce overwhelming cellular responses, including potent and sustained T and B cell responses. Cellular responses induced by adenovirus include HLA expression, antigen presentation, release of cytokines, and inflammatory changes in the infected cells and surrounding tissues, that collectively stimulate an anti-virus immune response [38]. Modification of the virus via insertion of immunoregulatory genes into the E3 region [40], which is not essential for viral replication [41,42], would induce anti-cancer immune response. Because the CRAd selectively replicates in CA-125-high cancer cells, not in normal cells, it would outperform other oncolytic adenoviruses for its ability to induce a potent and targeted anti-cancer immune response.

Oncolytic viruses eradiate cancer cells by two major distinct mechanisms: oncolytic activity that directly lyse infected cells, and anti-cancer immune response indirectly induced by virus infection of cancer cells. To date, none of the available oncolytic viruses, FDA-approved (18) or those under investigation (19-22), have been potent enough to eradicate all cancer cells in animals or patients. Enhancement of oncolytic activity is one of the main future directions in this field, however off-target toxicity is a concern. Development of oncolytic viruses with selective replication capacity in cancer cells and not in normal cells is reassuring. The 1040-bp promoter, which showed similar or higher transcriptional activity compared to E1A promoter in Hela cell, a cervical cancer cell line with a relatively high CA-125 level, but limited activity A549, a lung adenocarcinoma cell line, frequently used for adenovirus amplification. Therefore, Applicant believed that the replication capacity of Ad5/MUC16-1040 series was expected to be higher in CA-125-high cells than the wild-type adenovirus, but with limited replication in CA-125-low cells. Applicant further refined the 1040-bp promoter (SEQ ID NO:1) to a 646-bp promoter (SEQ ID NO:2) by removing 394-bp fragment that may contain transcription repression element. This 646-bp promoter showed 6-7 times higher in terms of transcriptional activity in Hela cells (CA-125-high cervical cancer cell line, which was used as a representative cell line because it is easy to grow and transfect with exogenous genes) than the 1040-bp promoter (FIG. 2 left, the 5^{th} set of bars vs the 7^{th} set of bars; FIG. 2 right, the 2^{nd} set of bars and the 3^{rd} set of bars), 12 times higher than the adenovirus E1A promoter in Hela cells. However, in A549 cells, it showed limited activity, only 59% of the E1A promoter. Applicant constructed an adenovirus with the 646-bp promoter (SEQ ID NO:2) to drive expression of E1A, which is anticipated to be more potent than the wild-type adenovirus to lyse ovarian cancer cells but with a favorable toxic profile.

Of interest, injection of virus-infected cells generally yielded a better outcome with prolonged survival than direct virus particle injection (FIG. 12D). One explanation may be that the infected cells are retained in the abdomen as virus carriers and held adjacent to the pre-inoculated cancer cells. Upon lysis, viruses may then be released to infect the adjacent cancer cells. Conversely, directly injected virus may be rapidly cleared within a few minutes [29], possibly by diffusion to circulation, neutralization by pre-existed antibodies, or absorption by cells lining the peritoneum. It is practical to obtain cancer cells from ovarian cancer patients with ascites, infect the collected cells with the virus, and return the infected cells back to abdomen as a virus carrier. Alternatively, the infected cancer cells may serve as a whole cell vaccine that can be used to stimulate an anti-cancer immune response to eradicate other cancer cells.

Adenovirus, either replication-competent or -deficient, is relatively safe and well tolerated in experimental animal models and clinical trials [43,44]. However, hepatotoxicity is one potentially life-threatening reaction to adenovirus delivery [44,45]. In the present disclosure, Applicant did not observe hepatotoxicity or any other noticeable side effects when mice were injected intraperitoneally at a dose of 10⁹ pfu/mouse. As the virus can only replicate in CA-125-expressing cells, the CRAd has a more favorable toxicity profile that may facilitate its translation from the laboratory into clinical practice.

Various immunotherapeutic approaches show promising efficacy for treatment of many tumor types, but not for ovarian cancer, except in the small subset of ovarian cancers with special gene mutations (23, 24), although mutation frequency in ovarian cancer is not low (25), compared to other types of human cancers. One reason may be that the cancer antigen in ovarian cancer cells is not well presented. Virus infection induces overwhelming change of the host cells and microenvironment such as antigen presentation, cytokine production, immune cell interaction etc. Results robustly demonstrated that mouse ID8 ovarian cancer cells infected with adenovirus that includes GM-CSF in the expression cassette in the E3 region induce an anti-cancer immune response and further inhibits growth of ID8 cells in re-challenge experiments. Mouse ID8 is a poor immunogenic ovarian cancer cell line that rarely induces a protective immune response to ID8 re-challenge in literature (26-28). The protective effect is more robust with STOSE cell line, another mouse ovarian cancer cell line, which was reported to be more immunogenic than ID8 (27, 28). In fact, anticancer immune-inducing capacity was demonstrated even when the STOSE cells were infected with the CRAd without GM-CSF. Thus, Applicant's data show anti-cancer immunity can be induced in ovarian cancer. Further, this approach represents a new opportunity for effective ovarian cancer immunotherapy.

The data presented herein challenges the dogma that CA-125 can only serve as biomarker of disease progression and treatment response, and it provides new hope that targeted therapy may yet be possible for ovarian cancer patients. Applicant took advantage of the specific transactivation of MUC16 and explore the feasibility and practicability of targeted therapy by developing an oncolytic virus that specifically replicates in and lyses ovarian cancer cells, and additionally induces the specific and potent anti-cancer immune response for ovarian cancer treatment.

### Example 3: Materials and Methods

Cell Lines and Culture

Ovarian cancer cell lines, CAOV3, HEYA8, OVCAR4, OVCAR8, PEO4, and TOV-112D, a mouse ovarian cancer cell line, ID8, a human embryonic kidney cell line, HEK 293, a human cervical cancer cell line, HeLa, and a human fibroblast cell line derived from ovarian cancer tissue, OVFB-1, were cultured in Dulbecco's Modified Eagle Medium (DMEM) (Corning Cellgro), supplemented with 10% Fetal Bovine Serum (Biowest) and 1× penicillin-streptomycin (Corning), in a humidified incubator (5% CO₂) at 37° C. Other ovarian cancer cell lines, A2780, IGROV-1, Kuramochi, OVCAR3, OVCAR5, and SKOV3, a human lung adenocarcinoma cell line, A549, and a human immortalized bronchial epithelial cell line, BEAS-2B, were cultured in RPMI 1640 (Corning Cellgro), supplemented with 10% Fetal Bovine Serum and 1× penicillin-streptomycin, in a humidified incubator (5% CO₂) at 37° C.

Cell Viability Assay

ID8 cells labeled with a stable firefly luciferase gene were plated in 96-well plates (5000 cell/well) and cultured overnight. Ad5/MUC16-1040/TK-EGFP were added at a multiplicity of infection (MOI) of 20:1. Ganciclovir (GCV; Sigma-Aldrich) was then added to the cells at a final concentration of 10 μM and incubated for an additional 3 days. D-luciferin (potassium salt; Syd Labs, MA) was added to each well at a final concentration of 100 μg/ml. Luminescence intensity was measured by a Tecan Spark 10M multimode micro-plate reader.

Alternatively, Applicant used Coomassie blue stain to visualize live cells attached to culturing plate after virus infection. Infected and died cells were floated and washed out with PBS. Attached live cells were stained with 0.1% Coomassie Brilliant Blue R-250 (Research Products International) in 50% methanol and 10% glacial acetic acid for 10 min. The plate wells were washes 3 times with tab water and dried for image scan.

Primary Ovarian Cancer Cell Collection and Culture

Primary ovarian cancer cell collection from patient ascites or pleural effusion was approved by the City of Hope Institutional Review Board (IRB; #07047). Patients were consented prior to fluid collection per the IRB-approved protocol. Laboratory use of the deidentified primary cells was approved under COH IRB #17478.

A portion of the collected fluid was sent for cytology evaluation, including examining the direct smear, cytospin, and cellblock slides using Papanicolaou stain, Diff-Quik stain, and H&E stain, per standard methods. Immunohistochemistry for expression of CA-125 (Clone OC125, Cell Marque), p53 (Clone BP53-11, Ventana), PAX8 (Clone MRQ-50, Ventana), WT-1 (Clone 6F-H2, Ventana), and calretinin (Clone SP65, Ventana) was performed to confirm diagnosis.

Live cells in the collected fluid were spun down and washed once by PBS. Red cells were removed by ammonium chloride buffer. Nucleated cells, including cancer cells (isolated single cells or cell clusters), immune cells, epithelial cells, mesothelial cells, and other cell types were suspended in DMEM with high glucose, supplemented with 10% FBS, 20 mM L-glutamine, 1× insulin-transferrin-selenium solution (GenDEPOT), and 1× penicillin-streptomycin, seeded in tissue culture dishes or plates, and cultured in a humidified incubator with 5% CO₂ at 37° C. The next day, floating cells were washed out gently, and CRAd was added to the cells at an MOI of 100:1. Infected cells were observed under an inverted phase contrast laser microscope, and images were captured with the integrated digital camera.

Adenovirus Construction, Amplification, and Purification

The AdenoQuick 2.0 system (OD260 Inc.), which consists of four shuttle vectors (pAd1127, pAd1128, pAd1129, and pAd1130), was used for adenovirus engineering. Each vector contains part of the wild-type human Ad5 genome that can be genetically modified and recombined for adenovirus packaging in HEK293 cells. pAd1127 contains the left end of the Ad5 genome with an E1A expression cassette. Applicant replaced the E1A promoter with the 1040 bp upstream DNA fragment of MUC16 with promoter activity in pAd1127, where E1A is an essential gene for adenovirus replication [18-20]. In the E3 region of pAd1129, applicant inserted a fusion gene, TK-EGFP, which encodes a fusion protein of EGFP and herpes simplex virus-1 thymidine kinase (HSV-TK). Construct sequences were verified by restriction digestion and sequencing, respectively. The adenovirus genome fragments from the four vectors were collected and ligated to generate full-length adenovirus genomic DNA with the inserts of interest, which was subsequently packaged into lambda phage heads for amplification in E. Coli. The recombined adenovirus genome DNA was exercised and transfected to HEK293 cells for packaging, per the manufacturer's instructions. Recombined adenoviruses were amplified in HEK293 cells and purified by cesium chloride ultracentrifugation. Purified viruses were stored in GTS buffer (2.5% glycerol, 25 mM NaCl, and 20 mM Tris-HCl, pH 8.0), per the manufacturer's instructions.

Reporter Vector Construction and Dual-Luciferase Assay

The Homo sapiens mucin 16, cell surface associated, (MUC16) gene sequence was retrieved from GenBank (NG 055257). Upstream DNA fragments of MUC16 were amplified by high-fidelity PCR and cloned into a reporter vector, pGL4.14 (Promega), to control expression of the built-in firefly luciferase gene. Reporter plasmids were transfected into the appropriate cultured cells, and transcriptional activity was measured using the Dual-Luciferase® Reporter Assay System (Promega), per the manufacturer's instructions.

mRNA Isolation and Quantitative Real Time PCR (qRT-PCR)

Total RNA was extracted using Qiagen's RNeasy Mini Kit, and complementary DNA was synthesized using Quantabio's qScript cDNA SuperMix Kit. Real-time PCR was performed using Applied Biosystems' Power SYBR Green PCR Master Mix and ABI Prism 7900HT Sequence Detection System. TheMUC16 primers were as follows: (sense) 5′-ACAAACTAGCAAAATAGGCTGT-3′ (SEQ ID NO:4) and (antisense) 5′-CTTCTTAATGTTTTTGGCATCT-3′ (SEQ ID NO:5). The threshold cycle number (Ct) for gene expression was calculated, and GAPDH was used as an internal control as follows: (sense) 5′-AAGAAGGTGGTGAAGCAGGC-3′ (SEQ ID NO:6) and (antisense) 5′-TCCACCACCCTGTTGCTGTA-3′ (SEQ ID NO:7). Relative fold induction was derived from the ΔΔCt values for gene expression.

Virus Oncolytic Activity in NSG Tumor Xenograft Models

All animal experiments were approved by the City of Hope Institution Animal Care and Use Committee (IACUC, #18013). Per the IACUC-approved protocol, female NSG mice (6-8 weeks old) were purchased from Jackson Laboratory and acclimated 1 week before being started on any study. Kuramochi/FL cells (5×10⁶) in 100 μL of PBS were injected intraperitoneally (ip) into mice on day 0. Ad5/MUC16-1040/TK-EGFP was delivered at a dose of 10⁹ pfu/mouse on day 1, as was the vehicle (buffer) control. For the OVCAR4 model, 3×10⁶ cells in 100 μL of PBS were injected ip on day 0, virus was delivered ip at 10⁹ pfu/mouse on day 8, as was the vehicle (buffer) control. An additional group was included in the OVCAR4 model; specifically, Applicant treated the OVCAR4 tumor-bearing mice with ip injection of 3×10⁵ OVCAR4 cells infected with the virus at MOI 100:1 on day 8. Mice were monitored closely and euthanized when cancer-related symptoms developed, such as weight loss, dehydration, abdomen distention, and impaired ambulation, per the IACUC-approved protocol.

Bioluminescent Imaging of Tumor-Bearing Mice

Mice bearing ovarian cancer cells labeled with firefly luciferase were imaged at indicated timepoints utilizing Spectral Instruments' SPECTRAL Lago X Imaging System. Specifically, each mouse received an intraperitoneal injection of 2 mg D-luciferin in 0.1 ml of deionized water, bioluminescent images were captured, and intensity was quantified using Spectral Instruments' companion Aura Imaging Software.

Statistical Analysis

Data collected from in vitro and in vivo experiments were analyzed by standard statistical methods, including student's t-test, ANOVA test, and Kaplan-Meier survival analysis using GraphPad Prism.

### Example 4: MUC16 Promoter Containing Oncolytic Viruses Including Immunoregulatory Genes

Immunotherapy is now well established as an effective cancer therapy for a wide variety of hematologic and solid malignancies; however, no immunotherapy has been approved specifically for the treatment of ovarian cancer. Immune checkpoint inhibitors have been tested in clinical trials for treatment of ovarian cancer at various stages as single agent or in combination with standard chemotherapy, anti-angiogenesis, or PARP inhibitors (4, 5). However, there has been only limited survival benefit supporting use of immune checkpoint inhibitors in ovarian cancer. Ovarian cancer vaccines have also been developed, including genetically modified whole cell vaccine (6, 7), dendritic cell vaccine (8), and peptide vaccines (9); however, limited clinical benefit has been reported. Yet abundant evidence indicates that ovarian cancer cells display neoantigens that can be recognized by immune cells (10-14). Likewise, tumor-infiltrating lymphocytes have been isolated from ovarian cancer tissues for use in adoptive experimental immunotherapy (15). Thus, the generally held explanation for the underwhelming performance of immunotherapy against ovarian cancer is ineffective expression or presentation of neoantigens and/or to the effects of an immunosuppressive tumor microenvironment (TME) (11, 12, 16).

Overcoming Ovarian Cancer Cell Immune Evasion would Bear Great Clinical Significance

Oncolytic virus infection of cancer cells represents an effective means of exposing cancer neoantigens to immune cells and disrupting a suppressive TME, as evidenced by the success of talimogene laherparepvec (T-VEC) for the treatment of melanoma (17, 18). Virus infection induces overwhelming molecular changes in cancer cells, including changes in gene expression, cytokine and chemokine production, antigen presentation, and cell survival and/or cell death pathways, that facilitate host defense against virus infection. Immune cells, including macrophages, NK cells, T cells, and B cells, are recruited to virus infected cells/tissues where they induce inflammatory changes in the TME that are intended to fight and clear the virus. At the same time, tumor antigens may be exposed and presented to activate an anti-cancer immune response that will be amplified by the immune stimulatory TME induced by virus infection. Thus, an oncolytic virus that specifically replicates in ovarian cancer cells, not affecting immune cells or other normal cells, would elicit an anti-cancer immune response recently termed “oncolytic immunotherapy”; the immune response will be robust if rationally-selected immunoregulatory genes are packaged within the oncolytic virus.

A unique feature of ovarian cancer is that more than 80% of patients express a high serum level of CA-125, one of the largest, hyperglycosylated, human proteins expressed in and shed from ovarian cancer cells. CA-125 levels in ovarian cancer patients can reach up to hundreds and thousands of units per milliliter (U/ml), while most other cancer patients and individuals without cancer have a low level of CA-125, below 35 U/ml or undetectable (2, 19, 20). Bioinformatic analysis shows that CA-125 mRNA is also highly expressed in ovarian cancer cells, but not in most other cancer cells or in normal cells.

Since its discovery in 1981 (21), CA-125 has been regarded as an ideal target for ovarian cancer treatment (22); however, targeting CA-125 for ovarian cancer treatment has never been successful to date. CA-125-targeted antibodies, such as Oregovomab, have been developed but showed no clinical advantage in large randomized placebo-controlled trials (23-26); although an immune response was observed (27). Adoptive T cell therapy using CA125-directed chimeric antigen receptors has also been developed for ovarian cancer (28), and a phase I trial has been proposed (29), but no further information has ever been reported.

It remains unknown why CA-125 is highly expressed in gynecological cancer cells, especially in ovarian cancer cells, and not in non-gynecological cancer cells or normal cells. The gene coding for CA-125, termed MUC16, is located on chromosome 19p13.2 and is comprised of approximately 179 kb of genomic DNA (22, 30). Specific transcriptional activation of the MUC16 gene in ovarian cancer cells is poorly defined (31, 32), and targeting specific MUC16 transactivation for ovarian cancer treatment has never been attempted. By identifying the MUC16 transactivation elements that control CA-125 transcription, Applicant was able to engineer a conditionally replicative adenovirus (CRAd) that displays high oncolytic activity in CA-125-expressing cancer cells (33). In preliminary studies, the CRAd also elicited host immune responses in a mouse ovarian cancer model. Applicant further armed the CRAd with immunoregulatory genes individually (CRAd/i) or in combination (CRAd/i+), anticipating that the process of specific virus infection and replication in ovarian cancer cells will actuate expression, release, and presentation of neoantigens to immune cells and will activate and amplify an effective anti-cancer immune response.

Experimental Approach

Described herein is a conditionally replicative adenovirus (CRAd) that displays high oncolytic activity in CA-125-expressing cancer cells (33). Results show that the CRAd also elicited host immune responses in a mouse ovarian cancer model. Applicant further armed the CRAd with immunoregulatory genes individually (CRAd/i) or in combination (CRAd/i+), to investigate whether the process of specific virus infection and replication in ovarian cancer cells actuated expression, release, and presentation of neoantigens to immune cells and activates and amplify an effective anti-cancer immune response.

Among all the oncolytic viruses, Applicant chose adenovirus as the platform for testing. Among other reasons described herein, adenovirus is one of the most common viruses, and it has been well characterized, demonstrating a safe toxic profile and a broad spectrum of targeted cells. Adenovirus can induce overwhelming cellular responses, such as HLA expression and antigen presentation, release of cytokines, inflammatory changes in the infected cells and surrounding tissues, and stimulation of an anti-virus immune response, which likely induces an anti-cancer immune response simultaneously.

By partnering a CRAd dependent on MUC16 transactivation for replication with immunomodulatory genes to alter the suppressive TME and to elicit a specific and potent anti-cancer immune response, Applicant sought to provide proof-of-concept that this oncolytic immunotherapy strategy can be targeted and effective in ovarian cancer. Refining the CRAd/i platform and performing more detailed mechanistic investigations of MUC16/CA-125 regulation in ovarian cancers cells and the oncolytic and immune activity induced by virus infection also facilitated the search for novel targets that can be exploited for future ovarian cancer treatments.

Various immunotherapeutic approaches have proven efficacious for the treatment of many cancer types, but none specifically for ovarian cancer (37, 38). Although mutation frequency in ovarian cancer is not low relative to many other human cancers (39). One explanation is that ovarian cancer neoantigens are simply not well exposed or presented to immune cells (12). Oncolytic virus infection is anticipated to induce overwhelming changes in target cells and the “cold” TME, likely inducing both anti-virus and anti-cancer immune responses and rendering the TME “hot”. The novel approach of utilizing CRAd/i armed with immunoregulatory genes to specifically replicate in ovarian cancer cells, not harming normal cells, and to simultaneously activate immune cells and enhance antigen presentation may effectively overcome this barrier to successful immunotherapy for ovarian cancer.

The 1040-bp MUC16 transactivation sequence shows similar or higher transcriptional activity compared to the E1A promoter in Hela cells, a cervical cancer cell line with a high CA-125 level, but limited activity in A549, a lung adenocarcinoma cell line, which is routinely used for adenovirus amplification. Therefore, Applicant anticipated that the replication capacity of CRAd would be higher in CA-125-high cells than the wild-type adenovirus with limited or no replication in CA-125-low cells, thereby addressing the specificity concern. Applicant also shows data indicating that liver toxicity is not observed utilizing the CRAd/i platform.

As described herein, Applicant identified two mouse ovarian cancer cells lines, ID8 and STOSE, which MUC16 promoter containing virus can infect, replicate in, and lyse. This makes it possible to evaluate whether CRAd/i infection of ovarian cancer cells can induce specific anti-cancer immune responses. Further, the CRAd/i platform has the benefit of being testable in vivo, and the preliminary data point to the feasibility and efficacy of this approach to targeted oncolytic immunotherapy for ovarian cancer treatment.

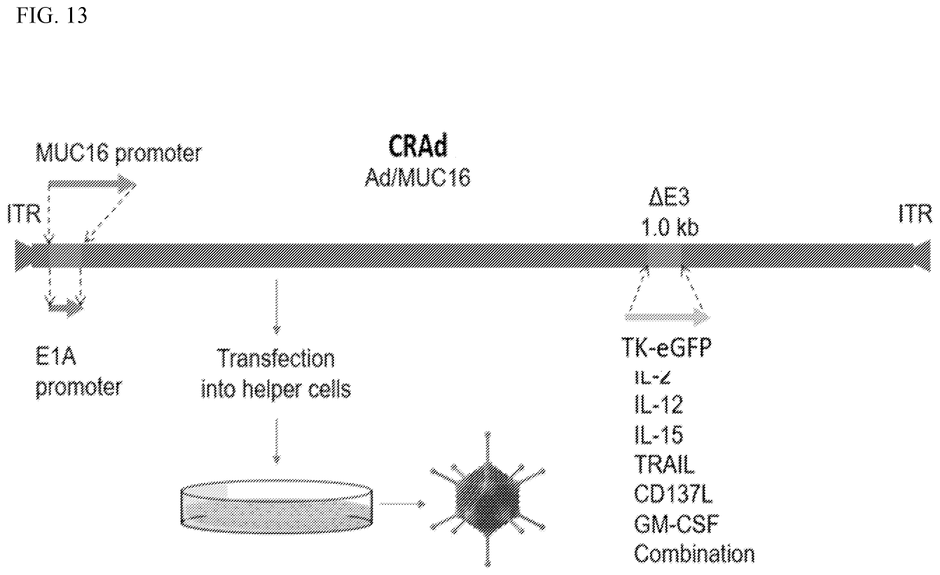

Applicant initiated adenovirus construction using this 1040-bp fragment and has made first generation CRAd that was used as a platform for testing the feasibility and efficacy of the oncolytic immunotherapy approach for ovarian cancer. Applicant used the AdenoQuick2.0 system (OD260 Inc., Boise, ID), which consists of four shuttle vectors (pAd1127, pAd1128, pAd1129, and pAd1130), where each vector contains part of the wild-type human adenovirus type 5 (Ad5) genome that can be genetically modified easily and then recombined for adenovirus packaging in HEK293 cells. Details of the shuttle vectors are listed in the Table 1. The virus construction with the MUC16-1040 bp promoter was then initiated, replacing the E1A promoter in pAd1127, where E1A is an essential gene for adenovirus replication (47-49). At the same time, the pAd1129 shuttle vector to express TK-eGFP at the E3 region was constructed. TK-eGFP is a fusion protein with both active eGFP that can be used to track virus infection and herpes simplex virus-1 thymidine kinase (HSV-TK) activity that can convert pro-drug, ganciclovir or other analogues, into a toxic metabolite to kill cells (50, 51). Applicant then digested and collected all adenovirus genome fragments from the 4 vectors and ligated them to generate a full-length adenovirus genomic DNA with inserts of interest, which was subsequently packaged into lambda phage heads for amplification in E. Coli. Finally, Applicant excised the recombined adenovirus genome DNA and transfected it into HEK293 cells, a human embryonic kidney cell line immortalized by Ad5 E1A/B that is routinely used for adenovirus packaging and amplification, because it expresses abundant E1A protein in the cells (52). In the transfected HEK293 cells, a a recombined adenovirus was successfully generated that Applicant termed as Ad5/MUC16/TK-eGFP, as illustrated in FIG. 13.

TABLE 1
Conditionally replicative adenovirus construction:
AdenoQuick2.0 system (OD260 Inc., Boise, ID).

| | | Insert (packaging | | Length: |
|---|---|---|---|---|
| Shuttle | Components | capacity 11.2 Kb) | | Position |
| pAd1127 | Left ITR, packaging | 1. | MUC16-1040 | 1040 bp: |
| | signal, the E1a and | | promoter | 425-1464 |
| | E1b regions, and the | 2. | Further refined | |
| | pIX coding sequence | | MUC16 promoter | |
| pAd1128 | E2 region (DNA | | | |
| | polymerase, terminal | | | |
| | protein, DNA-binding | | | |
| | protein), and many of | | | |
| | the late genes | | | |
| pAd1129 | E3 region, adenovirus | 1. | TK-eGFP | 1860 bp |
| | death protein (ADP), | 2. | CMV-GM-CSF- | 1130 bp |
| | and fiber gene | | Poly A | 1630 bp |
| | | 3. | CMV-CD137L- | 1170 bp |
| | | | Poly A | 2450 bp |
| | | 4. | CMV-IL-2- Poly A | 1190 bp |
| | | 5. | CMV-IL-12 (ab)- | 1550 bp |
| | | | Poly A | 2.5-5.0 |
| | | 6. | CMV-IL-15- Poly A | kb |
| | | 7. | CMV-TRAIL- | |
| | | | Poly A | |
| | | 8. | Combination up to | |
| | | | 3 in 1 cassette | |
| pAd1130 | E4 region and right | | | |
| | ITR | | | |

The Ad5/MUC16/TK-eGFP virus was found to replicate in and lyse CA-125 high-expressing cancer cells and does not replicate well in the CA-125 low or negative cancer cell lines (33). Further, Applicant tested it on a few normal cell lines, including human foreskin fibroblast, HFF, human lung epithelial cell line, BEAS21B, and two human fibroblast cell lines derived from human ovarian cancer established in the lab. As expected, Ad5/MUC16/TK-eGFP does not induce apparent CPE in these normal cells at an MOI up to 100:1 after a period of 2 weeks in culture with regular passaging every 3-7 days, although Applicant can see EGFP signal 2-3 days after infection, which gradually disappears with cell passaging. Conversely, in the virus-sensitive cells, infection with minimal virus would destroy the entire culture within 1-2 weeks. These results indicate that the virus can replicate in and lyse cancer cells expressing CA-125, not in cancer cell without CA-125 expression or normal cells.

Develop a Panel of CRAd/i Candidates and Evaluate their Anti-Cancer Effects In Vitro and In Vivo

Applicant further confirms the oncolytic activity of CRAd armed with immunomodulatory genes (CRAd/i) utilizing both ovarian cancer cell lines and primary ovarian cancer cells from patients. Test are performed on CRAd/i in ovarian cancer xenograft mouse models in NOD-scid IL2Rgammanu^{null} (NSG) immunodeficient mice to assess in vivo oncolytic activity. More CRAd/i are designed and constructed on the existing CRAd backbone, focusing on the selection of the immunoregulatory genes and gene combinations, where up to three genes are packaged into one virus (CRAd/i+) to boost and sustain host immune responses.

Arm the CRAd with Immunoregulatory Genes and Rational Combinations

Applicant chooses a series of immunoregulatory genes as listed in Table 1 and FIG. 13 for insertion into the E3 region of CRAd backbone. To date, Applicant has constructed three CRAd/i, carrying human IL-2 (Ad/MUC16/hIL-2), mouse GM-CSF (Ad/MUC16/mGM-CSF), and mouse CD137L (Ad/MUC16/mCD137L), which have yielded promising preliminarily results (described below). The panel of CRAd/i are expanded, and other immunoregulatory genes inserted, individually or in combination, and the generated CRAd/I are tested for their capacity to induce anti-cancer immune responses.

Importantly, the selection of immunoregulatory genes is based on their biological function, literature review, and the prior experiences. Briefly, GM-CSF functions to recruit antigen presenting cells to the tumor microenvironment, enhance dendritic cell function, and promote cytotoxic T-cell responses to tumor-associated antigens (53, 54). GM-CSF is the only gene for cancer gene therapy coupled with an HSV vector (T-VEC) that has ever been approved for treatment of melanoma (17, 18). IL-2 has long been investigated for activation and amplification of immune cells, mainly NK cells and T cells. IL-2 itself (55, 56) and IL-2-activated and -amplified lymphocytes, LAK cells (57, 58), and TIL cells (59), have been shown effective in cancer adaptive therapy. IL-12 is of particular interest because it stimulates T cell and natural killer cell activity, induces interferon gamma production (60), and suppresses immunosuppressive cells (61-63). IL-15 is another interleukin that induces proliferation and cytotoxicity of CD8+ cytotoxic and memory T cells, plus NK cells, with minimal induction of Treg cells (64, 65). CD137L is a transmembrane cytokine that belongs to the tumor necrosis factor (TNF) ligand family, which binds to a costimulatory receptor molecule, CD137 (also termed as 4-1BB, which has been used as a key component of the engineered T cell receptor of CAR-T cells) in T lymphocytes required for the optimal CD8 responses (66-68). TRAIL is a type II membrane protein (281 amino acids) that signals apoptosis via the death domain-containing receptors in a variety of human tumors, but not in normal cells. The soluble form of TRAIL (sTRAIL, aa 114-281) has been intensively studied in preclinical models and clinical trials. Applicant's unpublished data indicate that normal cells stably expressing exogenous full-length TRAIL induce apoptosis of surrounding cancer cells in both TRAIL-sensitive and -resistant cells, resulting in impressive anti-cancer activity in vitro and in vivo.

Applicant employs these immunoregulatory genes in recombinant CRAd system to amplify gene expression with replication to hundreds and thousands copies in ovarian cancer cells to induce anti-cancer immune responses. Ultimately, up to three genes are chosen with strong immune stimulatory capacity and engineer them into one CRAd, namely CRAd/i+. A single virus packaged with a combination of immune regulatory genes acting on different phases of the host immune response would induce an overwhelming and durable anti-cancer effect that can be applied to clinic.

Evaluate the Oncolytic Activity of the Engineered CRAd in NSG Xenograft Mouse Models of Ovarian Cancer

For these in vivo studies, Applicant selects CA-125 high-expressing human ovarian cancer cell lines labeled with a firefly luciferase gene to track tumor growth. As the peritoneum is the primary site of ovarian cancer progression, Applicant inoculates cancer cells into the peritoneum of NSG mice to establish the tumor xenografts. Virus is delivered by two distinct routes—one is the classic direct injection of virus particles into the peritoneum, and the other is using virus-infected cells as carriers to deliver virus into the peritoneum.

Direct Delivery of Viral Particles to Peritoneum

Up to five ovarian cancer cell lines are selected and labeled with firefly luciferase to track tumor growth, inoculating intraperitoneally into NSG mice at 1-5×10⁶ cells/mouse to establish ovarian cancer xenograft in peritoneum. Then CRAd intraperitoneally is injected, up to 1×10⁸⁻¹⁰ plaque forming units (pfu) per mouse, and tumor growth is monitored with the LagoX Spectral Instruments Imaging System, recording survival data for statistical analysis.

Delivery of Virus-Infected Cells as Virus Carriers to Peritoneum

As the adenovirus particle is extremely small and the peritoneum cavity is large, especially in humans, virus injected directly into the peritoneum may disappear quickly, limiting cancer cell infection efficiency (69). Thus, virus-infected cancer cells are also injected which is carried and amplified the virus in the peritoneum and release virus upon maturation to infect other cancer cells. Intraperitoneal injection of ovarian cancer cells is practical clinically, as ovarian cancer cells can be easily obtained from ascites.

Alternative Strategies

For complete eradication of established tumors, Applicant's selection and optimization of immune gene combinations is shown to be beneficial. As discussed, Applicant selects and combines two to three immunoregulatory genes that function on different phases of immune response (e.g., GM-CSF combined with IL-2 and CD137L), packaging them into a single CRAd. The primary strategy for linking three selected genes is use of two IRES sequences, putting them in one expression cassette with a CMV promoter (one of the shorted mammalian expression promoters) and a SV40 polyA signal with a total length less than 2.5 kb (in the IL-2/GM-CSF/CD137L example) to maximize expression; the maximum packaging capacity the AdenoQuick 2.0 system is 11.2 kb. The expression cassette is inserted into the E3 region of the pAd1129 plasmid and then transfected to HEK293 cells to verify expression of all three genes. Alternatively, Applicant can express two shorter genes with an TRES linker in one expression cassette and a longer gene in another cassette, which would yield a total insert length of 3.2 kb (in the IL-2/GM-CSF/CD137L example), which is still well within the packaging capacity of adenovirus.

Evaluate the Anti-Cancer Immune Response Induced by Top Performing CRAd/i and Explore the Mechanism(s) in a Syngeneic Mouse Model of Ovarian Cancer

Applicant fortuitously identified two mouse ovarian cancer cells lines, ID8 and STOSE, which the virus can infect, replicate in, and lyse. This makes it possible for us to evaluate whether CRAd/i infection of ovarian cancer cells can induce specific anti-cancer immune responses.

ID8 is a well-established mouse ovarian cancer cell line derived from C57BL/6 mouse ovarian surface epithelial cells (MOSEC) (72). Mice receiving an intraperitoneal injection of ID8 cells develop malignant ascites and die in 3-4 weeks. Unfortunately, ID8 is poorly immunogenic, and it rarely induces a protective immune response against ID8 rechallenge, as described in various cancer immunotherapy studies summarized elsewhere (45, 73). The likely reason is low tumor mutation burden and limited mutated proteins that can be presented as immunogenic neoantigens to host immune cells (44). It is reported that systemic anti-tumor immunity can be elicited by vaccinia virus combined with PD-L1 blockade in a mouse MC38 colon cancer model, but no protective effect has ever been reported in the mouse ID8 ovarian cancer model (74).

STOSE is another mouse ovarian cancer cell line derived from the parental M0505 cell line that originated from a FVB/NJ mouse ovarian surface epithelium (73); Applicant received this cell line as a courtesy of Dr. Barbara Vanderhyden, Ottawa Hospital Research Institute. The STOSE line grows rapidly with a doubling time of 13 hours in vitro. Intraperitoneal injection of STOSE into syngeneic FVB/NJ mice gives rise to malignant ascites and intraperitoneal tumors with a histologic phenotype resembling human high-grade serous carcinoma (HGSC). STOSE cells grow faster than the ID8 cells (doubling time about 18 hours) in vitro, but they grow much more slowly in syngeneic FVB/NJ mice than ID8 cells grow in C57BL/6 mice, requiring 3-4 months to kill the mice. This indicates that other in vivo factors, including the host immune system, may suppresses the growth of STOSE cells, also indicating that it may serve as a better model to evaluate anti-cancer immune response.

Applicant first tested ID8 cells infected with Ad5/MUC16/TK-eGFP against two controls, a mitomycin C-treated non-infected cell control and a PBS non-cell or vehicle control. Applicant infected ID8 cells with Ad5/MUC16/TK-eGFP at an MOI of 100:1 and cultured the cells for 48 hr. Applicant injected 0.1 ml (5×10⁶ cells/ml) to female C57BL/6 mice subcutaneously, twice, one week apart. Two weeks after the second vaccination, ID8 cells labeled with a firefly luciferase gene (2×10⁶ cells in 0.1 ml PBS) were injected intraperitoneally into the mice. Unfortunately, Applicant failed to observe any significant difference in bioluminescent intensity representative of tumor growth, and there was no obvious difference in survival. All mice in the three groups died 3-4 weeks after the ID8/FL inoculation. From this preliminary test, Applicant learned that CRAd (without an immunoregulatory gene) infection of ID8 cells is not sufficient to activate an anti-cancer immune response and protect against a rechallenge with parental cells, most likely due to the poor immunogenicity of ID8 cells (44).

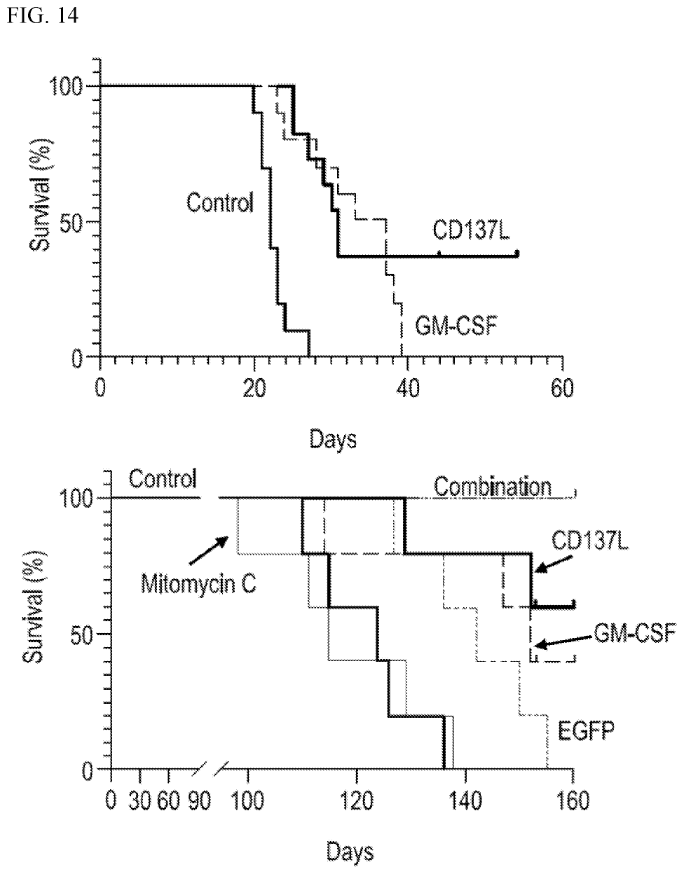

Thus, Applicant modified the strategy by arming the CRAd with immune regulatory genes. The first gene Applicant chose was mouse GM-CSF, one of best-known cytokines for cancer gene therapy that has been tested in many cancer types with both replication-deficient and -competent viruses, including the first and only US FDA-approved oncolytic virus, T-VEC, for the treatment of melanoma. Applicant also chose an immune stimulation signal protein, CD137L, the ligand of CD137, also termed 4-1BB, which has been used as a key component of the engineered T cell receptor of CAR-T cells. An expression cassette was generated with the mouse CMV promoter to control the ORF of mouse GM-CSF and CD137 and a SV40 ploy A signal to optimize expression. Applicant inserted the expression cassette into the E3 region of the CRAd and successfully generated two new viruses, Ad5/MUC16/mGM-CSF and Ad5/MUC16/mCD137L. Immunohistochemistry and ELISA confirmed expression of GM-CSF and CD137L in infected cells. Data demonstrated that, per the inoculation protocol described above, mice inoculated with ID8 cells infected with either Ad5/MUC16/mGM-CSF or Ad5/MUC16/mCD137L developed a robust anti-cancer immune response when rechallenged with parental ID8 cells, and they had significantly prolonged survival (FIG. 14 left, n=10, p<0.01). These results indicate that cancer cells with poor immunogenicity can be modified by CRAd/i to induce an obvious host immune response.

Applicant employed the STOSE mouse ovarian cell line to perform more detailed testing with the viruses. STOSE cells were infected with Ad5/MUC16/TK-eGFP (as a CRAd control), Ad5/MUC16/mGM-CSF, and Ad5/MUC16/mCD137L at an MOI of 100:1 and cultured for 48 hours. Applicant trypsinized the cells, washed the cells twice with PBS, resuspended in PBS, and injected into female FVB/NJ mice subcutaneously (1×10⁶ cells/mouse). Mitomycin C-treated (10 ug/ml for 2 hours) STOSE cells were used as a no CRAd infection control, and used PBS as a no cell control. One week later, the inoculation was repeated. Two weeks after the second vaccination, STOSE cells labeled with a firefly luciferase gene were injected intraperitoneally into the mice at 2×10⁶ cells in 0.1 ml PBS per mouse. Applicant monitored the tumor growth using the LagoX Spectral Instruments Imaging System. Although STOSE cells grow quickly in culture, it took much longer to develop ascites and grow tumors in the abdomen. Nevertheless, both control groups (mitomycin C and PBS) died or reached a point requiring euthanasia by IACUC protocol, while mice vaccinated with Ad5/MUC16/TK-eGFP survived longer (FIG. 14 bottom, n=5, p<0.01). Mice vaccinated with cells infected with Ad5/MUC16/mGM-CSF and Ad5/MUC16/mCD137L survived even longer than the Ad5/MUC16/TK-eGFP group, where 2 of 5 mice in the GM-CSF group (n=5, p<0.01) and 3 of 5 mice in the CD137L group (n=5, p<0.01) had survived for 8 months when finally euthanized. All 5 mice vaccinated with a combination of Ad5/MUC16/mGM-CSF and Ad5/MUC16/mCD137L survived for 8 months without evidence of residual disease, as indicated by lack of detection of firefly luciferase activity in the mice when euthanized.

Based on these data, Applicant arrived at three conclusions: 1) Virus-infected cancer cells can activate an immune response against tumor growth in rechallenge experiments (in the STOSE:FVB/NJ model); 2) Virus armed with either GM-CSF or CD137L can induce more potent immune responses in these rechallenge experiments (in both the ID8:C57Bl/6 and STOSE:FVB/NJ model); 3) Combination of GM-CSF and CD137 yields a protective immune response in the STOSE:FVB/NJ model that is synergistic in nature.

The injection route is further adjusted, scheduled, and dosed to maximize the anti-cancer capacity of the viruses. Assessment of the anti-cancer activity of these CRAd/i is used to guide clinical trials targeting ovarian cancer. New CRAd/i i carrying other immunoregulatory genes, as described herein, are generated and their capacity to induce an anti-cancer immune response in the syngeneic mouse model of ovarian cancer is demonstrated. Finally, CRAd/i expressing two or three immune genes in a single virus, or alternatively in a cocktail of three selected viruses each with a single immune gene, is tested.

Explore the Mechanism(s) of the Virus Infection-Induced Anti-Tumor Immune Response

Applicant has shown that virus infection induces specific anti-tumor immune responses. The following experiments are performed to confirm induction of specific cytotoxic T lymphocytes in the vaccinated mice.

T-Cell Depletion Assay

Specific antibodies are injected to deplete cytotoxic T lymphocytes: anti-CD4 (clone GK1.5, BioXCell) to deplete CD4+T helper (Th) cells, and anti-CD8-alpha (clone 2.43, BioXCell) to deplete CD8+ cytotoxic T lymphocytes (CTL) (75). Control mice received equal amounts of isotope control antibodies in equal doses. The ID8 mouse ovarian cancer model is used, because of shorter survival. First, mice is twice vaccinated with ID8 cells infected with Ad5/MUC16/mGM-CSF and Ad5/MUC16/mCD137, as described above. Two weeks after the second vaccination on day 0, ID8/FL cells are injected, 2×10⁶ cells in 0.1 ml PBS per mouse, intraperitoneally. Depletion antibodies are given on day −2 or −1, then continued injections one to three times a week per the reference protocol for each individual antibody (75). Applicant monitors tumor growth using the LagoX Spectral Instruments Imaging System, regularly recording bioluminescent intensity. The survival of mice in each group is also monitored. Applicant expects that depletion of either CD4+ or CD8+ cells compromises the protective immune response when mice are rechallenged with parental ID8 cells.

Cytotoxic T Lymphocyte (CTL) Assay

Splenocytes collected from C57BL/6 mice vaccinated with CARd/i-infected ID8 cells and control mice are cocultured with ID8 cells expressing firefly luciferase for 48 hours. All the cells are are spun down and resuspended in media supplemented with D-luciferin potassium salt and incubated at RT for 5 min. Bioluminescence intensity is measured by a TECAN plate reader. Similarly, Applicant collects splenocytes from FVB/NJ mice vaccinated with CRAd/i-infected STOSE cells and test for CTL activity again in the STOSE cells. Applicant uses STOSE cells as the non-specific control target for the C57BL/6:ID8 model and ID8 cells as the non-specific control for the FVB/NJ:STOSE model. Cytolytic activity is calculated using luciferase emission value. Killing lysis %=[1−(unknown−blank)/(positive control−blank)]×100% (76).

Cytokine Release Assay

Splenocytes are isolated from mice vaccinated with CRAd/i-infected cancer cells and cocultured with corresponding cancer cells in a U-bottom microplate for 18 hours (77). Supernatant is collected and subjected to cytokine tests, including IFN-gamma, IL-2, TNF-alpha, Granzyme B, and Perforin, using the LEGENDplex™ bead-based immunoassays: pre-defined mouse panels (BioLegend, San Diego, CA).

Flow Cytometry Assay for Sub-Population of Immune Cells

Mice vaccinated with CRAd/i-infected cancer cells are rechallenged with parental cells intraperitoneally. Two days later, cells are collected, washed with PBS, and stained with antibodies against CD45, CD8, CD4, PD-1, ICOS, CTLA-4, TIM-3, LAG-3, TIGIT, and Foxp3 to determine the quantities of CD8 T cells, activated CD8 T-cells, exhausted CD8 T-cells, and CD4 Treg cells in the TME. Applicant also tests the levels of mouse myeloid-derived suppressor cells (MDSC), both granulocytic (CD11b⁺ Gr-1/Ly-6G^{high} Ly-6C^{low}) and monocytic (CD11b+Gr-1/Ly-6G^{-/low} Ly-6C^{high}) cells.

Considerations

Adenovirus, either replication-competent or -deficient, is relatively safe and well tolerated in experimental animal models and clinical trials (78, 79). In the present studies, hepatotoxicity or any other noticeable side effects are not observed when mice were injected intraperitoneally at a dose of 10⁸ pfu/mouse daily for up to 5 days. As the virus can only replicate in CA-125-expressing cells, Applicant expects that recombinant CRAd should show less hepatoxicity. To confirm this, Applicant increases the dose up to 10¹⁰ pfu/mouse and compares it with wild-type Ad5 at the same doses. The mice are observed closely for toxicity and collect blood by retro-orbital bleeding to quantify the liver enzymes, ALT and AST, using a colorimetric aspartate aminotransferase activity assay kit and an alanine aminotransferase activity assay kit (both Sigma Aldrich), respectively. Replacement of the adenovirus E1A promoter with the MUC16 promoter is expected to make the virus inactive and therefore less toxic.

Alternative Strategies

The Ad5/MUC16/TK-eGFP shows excellent oncolytic activity in vitro. It can replicate in an infected cell and be released upon maturation to infect other cells until all cells die. To date, none of the oncolytic viruses, including the US FDA approved T-VEC, the Chinese FDA approved H101, and other oncolytic viruses under clinical and preclinical investigation, can eradicate an established tumor mass completely using the viruses themselves, likely due to the natural barriers (18, 81, 82). Complete responses induced by T-VEC in melanoma patients is largely due to subsequent immune responses induced by the virus-infected cancer cells. Applicant has demonstrated the oncolytic activity of the Ad5/MUC16 viruses, which 1) helped prove that the hypothesis to generate a virus using a MUC16 promoter to control virus replication for oncolysis is practical and 2) helped demonstrate a protective immune response in an immunocompetent syngeneic mouse model of ovarian cancer. The protective immune response can be further amplified by incorporation of the immunoregulatory genes GM-CSF and CD137L. As Applicant incorporates more of the immunoregulatory genes discussed herein into the CRAd platform, maximized vaccination capacity ultimately yielding an optimized and robust immune response is expected.

One practical alternative approach is combination with immune checkpoint inhibitors to further potentiate the immunogenicity of the virus-infected cancer cells. For this approach, Applicant incorporates immune checkpoint inhibitors to block PD-1/PD-L1 and/or CTLA-4/B7 interactions to reduce suppressive signals and enhance immune responses induced by virus-infected cancer cells. This potentially provides evidence of optimized combinations that can be used to guide future oncolytic immunotherapy clinical trials for ovarian cancer patients.

Assess the Oncolytic and Immunostimulatory Activity of Top-Performing CRAd/i in Primary Ovarian Cancer Cells and Immune Cells Collected from Patients

Investigations are conducted to determine whether the virus meets the requirement that they infect and replicate in human ovarian cancer cells without adversely affecting normal cells. Response of immune cells are determined in the CRAd/i-infected culture.

In Vitro Culturing of Primary Cells from Ascites or Pleural Effusion

With IRB approval and informed consent, Applicant collects (at a rate about 5 patients per year) one-time samples of ascites or pleural effusion from up to 20 patients diagnosed with ovarian cancer and a serum CA-125 level more than 500 U/ml. For the related experiments, primary ovarian cancer cells are cultured and immune cells from each patient donor as follows:

Ascites are filtered through a nylon mesh to remove tissue debris and large cell clumps. Red cell contamination is removed by ammonium chloride red cell lysis buffer. Under an inverted phase microscope, ovarian cancer cells are large, round, and bright, usually clustered in the form of spheroids that can be easily distinguished from other cells (FIG. 11A). Mesothelial cells and epithelial cells are usually attached to the culture surface overnight and are difficult to distinguish from other single isolated cancer cells. Cancer spheroids are attached to the culture surface to form a monolayer cell patch and then slowly spread to the surrounding area. Fibroblasts grow rapidly in serum-containing medium and occupies all available culture surface, but they stop growing when they become confluent. Lymphocytes interreact with other cells, attach to the culture surface, or float in the medium. They survive for a few days and gradually die if no stimulation signals are added to the culture.

In Vitro Infection of Primary Cells with CRAD/i Viruses

Twenty-four hours after aliquots of collected cells are seeded, baseline assessment is recorded, then virus (or control) is added at an MOI of 10:1. At baseline and again at 7 and 14 days, cells are observed and monitored for morphology change, proliferation, and EGFP expression, under an inverted laser microscope. Cell death is measured by CellTiter-Glo® 2.0 Cell Viability Assay (Promega, Madison, MI). Applicant has tested the CRAd, Ad/MUC16/TK-EGFP, in cells collected from pleural effusion or ascites of 4 ovarian cancer patients. The data indicates that the CRAd can replicate in the cancer cells, not normal cells as shown in a representative case in FIG. 11A. Primary cancer cells infected with Ad/MUC16/TK-EGFP showed classic CPE and expressed EGFP about five days after infection. Fourteen days after infection, cancer cells detached from cultured surface and died gradually. While fibroblasts, which are sensitive to wild-type adenovirus infection (83, 84), were not affected by the CRAd, which continued to grow and occupy the culture surface where tumor cells had been attached (FIG. 11A, lower panel). Applicant further confirms that the virus can only replicate in ovarian cancer cells by staining with antibodies against markers of ovarian cancer cells (anti-CA-125, anti-EpCAM, and anti-E-cadherin, all R&D Systems), lymphocytes (anti-CD45, BioLegend, San Diego, CA), mesothelial cells (anti-podoplanin, BioLegend), macrophage (anti-CD14 and anti-CD44, both BioLegend), fibroblasts (anti-FAP-alpha, R&D Systems), and epithelial cells (anti-EpCAM or anti-E-cadherin, both R&D Systems), together with anti-Ad5 hexon (Bio-Rad) and EGFP signal. Applicant is confident that recombinant CRAd has a target spectrum limited to CA-125-expressing cells.

Applicant also partially purifies the cancer spheroids by low-speed centrifugation and seed them in a round bottom ultra-low attachment 96-well plate to set up 3D cultures that can then be infected with CRAd. Applicant monitors the cancer organoid to determine whether the virus can kill primary cancer cells growing as 3D organoid, the in vitro mimic of a solid tumor mass. Virus infection is visualized by expression of EGFP. Cell death is detected using the CellTiter-Glo 3D Cell Viability Assay (Promega, Madison, MI) per the manufacturer's protocol.

Reaction of Immune Cells to Virus Infection

To understand the reaction of immune cells to virus infection in this culture environment, the cells collected from ovarian cancer ascites are infected with CRAd/I; the immune cells are then monitored for proliferation and activation by observation under a microscope and flow cytometry. Supernatant from the infected cells and test cytokine levels are collected. Applicant anticipates that CRAd/i infection of primary cells collected from ascites induce lymphocyte proliferation and activation, and profound cytokine production.

Applicant collects cells from the CRAd/i-infected primary cell culture, 7 days and 14 days after infection, and stain with antibodies specific to various immune cell subtypes and perform flow cytometry to quantify the cells that mediate cellular immune responses, such as helper T cells, effector CD8 T cells, NK cells, macrophages, dendritic cells. The changes in absolute number and fraction are quantified in the virus-infected culture compared to the non-viral infection control culture. Applicant expects that these immune effectors will increase in numbers and/or fractions. The cytotoxicity function of these cells is tested by intracellular cytokine staining to determine the number of IFN-gamma, granzyme B, and CD107a producing cells. Further, whether there is a change in the number of human MDSC will be investigated, both the granulocytic (Lin⁻ CD11b⁺ CD14⁻ CD15⁺ CD33⁺ CD66b⁺ HLA-DR⁻) and monocytic (Lin⁻ CD11b⁺ CD14⁺ CD15⁻ CD33⁺ CD66b⁻ HLA-DR⁻) subsets, and Treg cells (CD3⁺CD4⁺CD25⁺FoxP3⁺), the two major cell populations that constitute the suppressive abdominal TME of ovarian cancer (85-87). Applicant anticipates that virus infection induce overwhelming immune cell profile changes that favor of a positive host response.

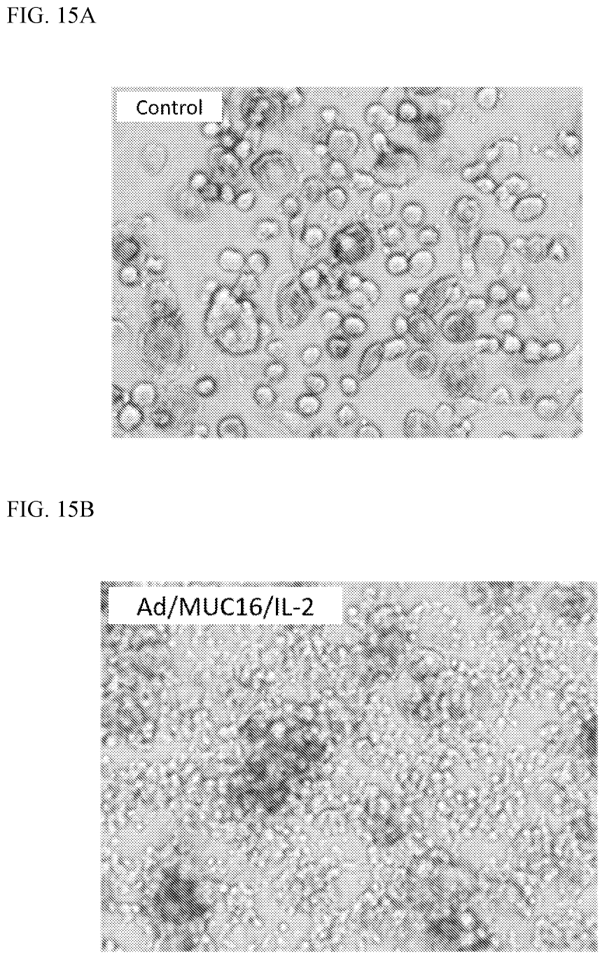

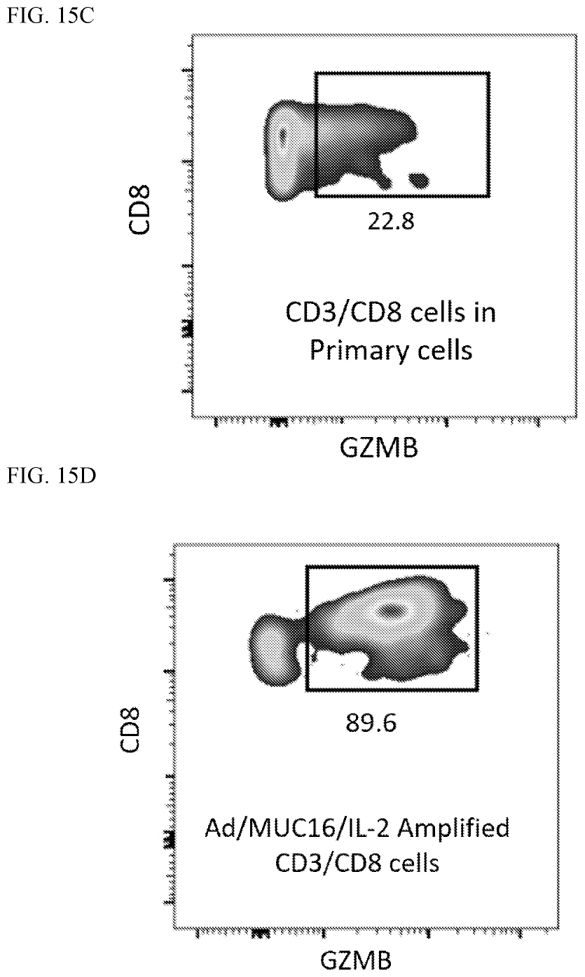

Applicant has tested one of theCRAd/i, Ad/MUC16/hIL-2, on primary cells collected from a patient. The cells were infected in culture with recombinant Ad/MUC16/hIL-2 at an MOI (multiplicity of infection) 10:1. One week later, significant amplification of lymphocytes were observed in the Ad/MUC16/hIL-2 infected wells as shown in FIGS. 15A and 15B. Preliminary data indicate that most of the amplified lymphocytes were T cells. In this case, CD3⁺ CD4⁺ (also FoxP3⁺, indicating Treg cells) cells are more numerous than the CD3⁺ CD8⁺ cells, both of which amplified hundreds and thousands of times in 10 days. Of particular interest, the majority of CD3⁺ CD8⁺ amplified with infection of Ad/MUC16/hIL-2 turned granzyme B positive, an indicator of activated CTL (FIGS. 15C and 15D). Applicant is further verifying cytotoxicity of the amplified CD3⁺ CD8⁺ cells. Results described herein are encouraging for the following reasons: 1) CRAd/i does specifically replicate in cancer cells, does not affect lymphocyte amplification; 2) The infected cancer cells express exogenous immunoregulatory cytokines, in this case, IL-2, which is functional in the in vitro microenvironment; 3) CRAd/i platform and models are ideal for investigating immune response to cancer cells genetically modified with immunoregulators.

At the same time, supernatant from the infected cells is collected and store in −80 for cytokine level test. Applicant uses LEGENDplex™ bead-based immunoassays: pre-defined human panels (BioLegend, San Diego, CA) to determine the levels of cytokines and highlight those directly regulate immune response such as IFN-gamma, IL-2, TNF-alpha, granzyme B, perforin, IL-12, IL-6, IL-10, and macrophage inflammatory proteins 1 (MIP-1) in the supernatant.

Applicant anticipates these viruses only replicate in CA-125 expressing cancer cells, thus they can be used as carriers for virus delivery, intraperitoneally or intratumorally, and as a vaccine to induce anti-cancer immune responses for ovarian cancer treatment. Via successful completion of the proposed study, Applicant anticipates generation of an oncolytic virus that specifically replicates in and kills ovarian cancer cells and induces protective anti-cancer immune responses in preclinical models. These IND-enabling studies advance the field and provides the data needed to propose a pilot clinical study using the oncolytic virus for ovarian cancer treatment.

Alternative Strategies

Applicant has made an essential step forward by establishing a platform whereby lymphocytes are impacted by CRAd-mediated expression of immunoregulatory genes in primary cancer cells. Preliminary data shows CD3⁺ CD8⁺ T cells, but also more CD3⁺ CD4⁺ cells (mainly FoxP3⁺ Treg cells) present in the malignant EOC effusion, consistent with the literature and with a poorer prognosis. However, CRAd-mediated expression of IL-2 in primary cancer cells amplified and activated CD3⁺ CD8⁺ T cells, represented by increased positivity of granzyme B, an indicator of functional CTLs. At the same time, CD3⁺ CD4⁺ FoxP3⁺ T cells were also amplified, because they express high level CD25, a high affinity receptor of IL-2. Additional cases are assessed, and amplified lymphocyates, cytotoxic activity and cytokine production profiles are further evaluated to determine if CRAd/i consistently promotes an anti-cancer immune response in primary human ovarian cancer cells. The next step is to overcome stimulation of suppressive cells and simultaneously stimulate cytokine production needed to activate a host-protective anti-cancer immune response. Applicant is constructing CRAd/i carrying other immunoregulatory genes, such as IL-12, IL-15, which are different from IL-2 and capable of inducing cytotoxic and memory T cells and inhibiting suppressive Treg cells and macrophages. Applicant can also employ a low-dose of cyclophosphamide (88), which is an effective agent for deletion of Treg cells. Further, antibodies to CTLA-4 (89, 90), CD25 (91), and PD-L1 (90) can be used to deplete or inhibit Treg cells and monitor anti-cancer immune responses (92). With all these measures, suppressive effects of the TME are minimized and the anti-cancer immune response induced by CRAd/i infection is maximized.

General Considerations Across the Statistical Analysis Plan:

Due to the multiplicity of treatment-versus-control comparisons per experiment, the False Discovery Rate method is used to limit to 5% or less the risk of statistical error from multiple hypothesis testing. Continuous endpoints may be log-transformed as appropriate to optimize statistical modeling. Whether an effect's slope or trajectory differs by treatment is investigated in the models using treatment-by-time interaction terms.

Statistical Analysis Plan:

In vivo experiments: Endpoints are survival and tumor burden over time. Survival under the various treatments are compared against control using proportional hazards regression or accelerated failure-time regression, as appropriate. Tumor growth under the various treatments are compared against control using mixed linear regression. Because tumor volume becomes non-randomly missing at death, it is necessary to model tumor growth jointly with survival.

In vitro experiments: Endpoints are cytolytic activity (luciferase emission value) and cytokine release (per immunoassay). The effects of the various treatment parameters (virus, route, schedule, dosage) are compared against control using generalized linear regression.

Tumor rechallenge: Endpoints are the absolute numbers and fractions of cells within each of several sub-populations of immune cells. The effects of the various treatments are compared against control using generalized linear regression models.

In vitro experiments: Endpoints include (for mixed cell cultures) morphology change, proliferation, and EGFP expression; cell viability of cancer organoid; and (for subsets of immune cells) proliferation, activation, and cytokine production. Endpoints are measured on Day 0 (pre-infection), 7, and 14 (shorter time interval for cytokines). Modeling appropriate for repeated measurements (mixed linear regression or generalized linear regression) is used to compare against control the effects of individual viruses at varying dosages. As appropriate, endpoints may be analyzed as change-from-Day 0 values. These experiments involve cells from up to 20 ovarian cancer patients. When data from multiple patients are analyzed jointly, correlation among data linked to the same donor is taken into account.

Data Analysis

Data collected from in vitro and in vivo experiments are analyzed by standard statistical methods described above, including student's t-test, ANOVA test, Pearson correlation coefficient, and Kaplan-Meier survival analysis using GraphPad Prism.

### REFERENCES FOR EXAMPLES 1-3

1. Board PDQATE. Ovarian Epithelial, Fallopian Tube, and Primary Peritoneal Cancer Treatment (PDQ(R)): Health Professional Version. PDQ Cancer Information Summaries. Bethesda (MD): National Cancer Institute (US); 2002.
2. Nebgen D R, Lu K H, Bast R C, Jr. Novel Approaches to Ovarian Cancer Screening. Current oncology reports. 2019; 21(8):75.
3. Siegel R L, Miller K D, Jemal A. Cancer statistics, 2018. CA: a cancer journal for clinicians. 2018; 68(1):7-30.
4. Dochez V, Caillon H, Vaucel E, Dimet J, Winer N, Ducarme G. Biomarkers and algorithms for diagnosis of ovarian cancer: CA125, HE4, RMI and ROMA, a review. Journal of ovarian research. 2019; 12(1):28.
5. Gandhi T, Bhatt H. Cancer Antigen 125. StatPearls. Treasure Island (FL): StatPearls Publishing Copyright © 2020, StatPearls Publishing LLC.; 2020.
6. Bast R C, Jr., Feeney M, Lazarus H, Nadler L M, Colvin R B, Knapp R C. Reactivity of a monoclonal antibody with human ovarian carcinoma. The Journal of clinical investigation. 1981; 68(5):1331-7.
7. Aithal A, Rauth S, Kshirsagar P, Shah A, Lakshmanan I, Junker W M, et al. MUC16 as a novel target for cancer therapy. Expert opinion on therapeutic targets. 2018; 22(8):675-86.
8. Liu J F, Moore K N, Birrer M J, Berlin S, Matulonis U A, Infante J R, et al. Phase I study of safety and pharmacokinetics of the anti-MUC16 antibody-drug conjugate DMUC5754A in patients with platinum-resistant ovarian cancer or unresectable pancreatic cancer. Annals of oncology: official journal of the European Society for Medical Oncology. 2016; 27(11):2124-30.
9. Paijens S T, Leffers N, Daemen T, Helfrich W, Boezen H M, Cohlen B J, et al. Antigen-specific active immunotherapy for ovarian cancer. The Cochrane database of systematic reviews. 2018; 9(9):Cd007287.
10. Sabbatini P, Harter P, Scambia G, Sehouli J, Meier W, Wimberger P, et al. Abagovomab as maintenance therapy in patients with epithelial ovarian cancer: a phase III trial of the AGO OVAR, COGI, GINECO, and GEICO—the MIMOSA study. Journal of clinical oncology: official journal of the American Society of Clinical Oncology. 2013; 31(12):1554-61.
11. Berek J S, Taylor P T, Gordon A, Cunningham M J, Finkler N, Orr J, Jr., et al. Randomized, placebo-controlled study of oregovomab for consolidation of clinical remission in patients with advanced ovarian cancer. Journal of clinical oncology: official journal of the American Society of Clinical Oncology. 2004; 22(17):3507-16.
12. Battaglia A, Fossati M, Buzzonetti A, Scambia G, Fattorossi A. A robust immune system conditions the response to abagovomab (anti-idiotypic monoclonal antibody mimicking the CA125 protein) vaccination in ovarian cancer patients. Immunology letters. 2017; 191:35-9.
13. Chekmasova A A, Rao T D, Nikhamin Y, Park K J, Levine D A, Spriggs D R, et al. Successful eradication of established peritoneal ovarian tumors in SCID-Beige mice following adoptive transfer of T cells genetically targeted to the MUC16 antigen. Clinical cancer research: an official journal of the American Association for Cancer Research. 2010; 16(14):3594-606.
14. Koneru M, O'Cearbhaill R, Pendharkar S, Spriggs D R, Brentjens R J. A phase I clinical trial of adoptive T cell therapy using IL-12 secreting MUC-16(ecto) directed chimeric antigen receptors for recurrent ovarian cancer. Journal of translational medicine. 2015; 13:102.
15. Yin B W, Lloyd K O. Molecular cloning of the CA125 ovarian cancer antigen: identification as a new mucin, MUC16. The Journal of biological chemistry. 2001; 276(29):27371-5.
16. Thapi D, Binder D, Rosales N, Yan X J, Lash A E, Spriggs D R. Abstract 2033: Transcription factors that regulate MUC16 expression in ovarian cancer. Cancer Research. 2011; 71(8 Supplement):2033-.
17. Zhang M X, Hong S S, Cai Q Q, Zhang M, Chen J, Zhang X Y, et al. Transcriptional control of the MUC16 promoter facilitates follicle-stimulating hormone peptide-conjugated shRNA nanoparticle-mediated inhibition of ovarian carcinoma in vivo. Drug delivery. 2018; 25(1):797-806.
18. Haitz K, Khosravi H, Lin J Y, Menge T, Nambudiri V E. Review of talimogene laherparepvec: A first-in-class oncolytic viral treatment of advanced melanoma. Journal of the American Academy of Dermatology. 2020; 83(1):189-96.
19. Vasey P A, Shulman L N, Campos S, Davis J, Gore M, Johnston S, et al. Phase I trial of intraperitoneal injection of the E1B-55-kd-gene-deleted adenovirus ONYX-015 (dl1520) given on days 1 through 5 every 3 weeks in patients with recurrent/refractory epithelial ovarian cancer. Journal of clinical oncology: official journal of the American Society of Clinical Oncology. 2002; 20(6):1562-9.
20. Bischoff J R, Kirn D H, Williams A, Heise C, Horn S, Muna M, et al. An adenovirus mutant that replicates selectively in p53-deficient human tumor cells. Science (New York, NY). 1996; 274(5286):373-6.
21. Sauthoff H, Hu J, Maca C, Goldman M, Heitner S, Yee H, et al. Intratumoral spread of wild-type adenovirus is limited after local injection of human xenograft tumors: virus persists and spreads systemically at late time points. Human gene therapy. 2003; 14(5):425-33.
22. Harrison D, Sauthoff H, Heitner S, Jagirdar J, Rom W N, Hay J G. Wild-type adenovirus decreases tumor xenograft growth, but despite viral persistence complete tumor responses are rarely achieved—deletion of the viral Elb-19-kD gene increases the viral oncolytic effect. Human gene therapy. 2001; 12(10):1323-32.
23. Brahmer J R, Tykodi S S, Chow L Q, Hwu W J, Topalian S L, Hwu P, et al. Safety and activity of anti-PD-L1 antibody in patients with advanced cancer. The New England journal of medicine. 2012; 366(26):2455-65.
24. Boyiadzis M M, Kirkwood J M, Marshall J L, Pritchard C C, Azad N S, Gulley J L. Significance and implications of FDA approval of pembrolizumab for biomarker-defined disease. Journal for immunotherapy of cancer. 2018; 6(1):35.
25. Lawrence M S, Stojanov P, Polak P, Kryukov G V, Cibulskis K, Sivachenko A, et al. Mutational heterogeneity in cancer and the search for new cancer-associated genes. Nature. 2013; 499(7457):214-8.
26. Martin S D, Brown S D, Wick D A, Nielsen J S, Kroeger D R, Twumasi-Boateng K, et al. Low Mutation Burden in Ovarian Cancer May Limit the Utility of Neoantigen-Targeted Vaccines. PloS one. 2016; 11(5):e0155189.
27. McCloskey C W, Rodriguez G M, Galpin K J C, Vanderhyden B C. Ovarian Cancer Immunotherapy: Preclinical Models and Emerging Therapeutics. Cancers. 2018; 10(8).
28. McCloskey C W, Goldberg R L, Carter L E, Gamwell L F, Al-Hujaily E M, Collins O, et al. A new spontaneously transformed syngeneic model of high-grade serous ovarian cancer with a tumor-initiating cell population. Frontiers in oncology. 2014; 4:53.
29. Flint J, Shenk T. Adenovirus E1A protein paradigm viral transactivator. Annual review of genetics. 1989; 23:141-61.
30. Frisch S M, Mymryk J S. Adenovirus-5 E1A: paradox and paradigm. Nature reviews Molecular cell biology. 2002; 3(6):441-52.
31. Moran E, Mathews M B. Multiple functional domains in the adenovirus E1A gene. Cell. 1987; 48(2):177-8.
32. Steffens S, Frank S, Fischer U, Heuser C, Meyer K L, Dobberstein K U, et al. Enhanced green fluorescent protein fusion proteins of herpes simplex virus type 1 thymidine kinase and cytochrome P450 4B1: applications for prodrug-activating gene therapy. Cancer gene therapy. 2000; 7(5):806-12.
33. Louis N, Evelegh C, Graham F L. Cloning and sequencing of the cellular-viral junctions from the human adenovirus type 5 transformed 293 cell line. Virology. 1997; 233(2):423-9.
34. Chesney J, Puzanov I, Collichio F, Singh P, Milhem M M, Glaspy J, et al. Randomized, Open-Label Phase I I Study Evaluating the Efficacy and Safety of Talimogene Laherparepvec in Combination With Ipilimumab Versus Ipilimumab Alone in Patients With Advanced, Unresectable Melanoma. Journal of clinical oncology: official journal of the American Society of Clinical Oncology. 2018; 36(17):1658-67.
35. Liang M. Oncorine, the World First Oncolytic Virus Medicine and its Update in China. Current cancer drug targets. 2018; 18(2):171-6.

### REFERENCES FOR EXAMPLE 4

1. Board PDQATE. Ovarian Epithelial, Fallopian Tube, and Primary Peritoneal Cancer Treatment (PDQ(R)): Health Professional Version. PDQ Cancer Information Summaries. Bethesda (MD): National Cancer Institute (US); 2002.
2. Nebgen D R, Lu K H, Bast R C, Jr. Novel Approaches to Ovarian Cancer Screening. Current oncology reports. 2019; 21(8):75.
3. Siegel R L, Miller K D, Jemal A. Cancer statistics, 2018. CA: a cancer journal for clinicians. 2018; 68(1):7-30.
4. Moore K N, Bookman M, Sehouli J, Miller A, Anderson C, Scambia G, Myers T, Taskiran C, Robison K, Mäenpää J, Willmott L, Colombo N, Thomes-Pepin J, Liontos M, Gold M A, Garcia Y, Sharma S K, Darus C J, Aghajanian C, Okamoto A, Wu X, Safin R, Wu F, Molinero L, Maiya V, Khor V K, Lin Y G, Pignata S. Atezolizumab, Bevacizumab, and Chemotherapy for Newly Diagnosed Stage III or IV Ovarian Cancer: Placebo-Controlled Randomized Phase III Trial (IMagyn050/GOG 3015/ENGOT-OV39). J Clin Oncol. 2021:Jco2100306.
5. Zamarin D, Burger R A, Sill M W, Powell D J, Jr., Lankes H A, Feldman M D, Zivanovic O, Gunderson C, Ko E, Mathews C, Sharma S, Hagemann A R, Khleif S, Aghajanian C. Randomized Phase I I Trial of Nivolumab Versus Nivolumab and Ipilimumab for Recurrent or Persistent Ovarian Cancer: An NRG Oncology Study. J Clin Oncol. 2020; 38(16):1814-23.
6. Rocconi R P, Grosen E A, Ghamande S A, Chan J K, Barve M A, Oh J, Tewari D, Morris P C, Stevens E E, Bottsford-Miller J N, Tang M, Aaron P, Stanbery L, Horvath S, Wallraven G, Bognar E, Manning L, Nemunaitis J, Shanahan D, Slomovitz B M, Herzog T J, Monk B J, Coleman R L. Gemogenovatucel-T (Vigil) immunotherapy as maintenance in frontline stage III/IV ovarian cancer (VITAL): a randomised, double-blind, placebo-controlled, phase 2b trial. Lancet Oncol. 2020; 21(12):1661-72.
7. Oh J, Barve M, Matthews C M, Koon E C, Heffernan T P, Fine B, Grosen E, Bergman M K, Fleming E L, DeMars L R, West L, Spitz D L, Goodman H, Hancock K C, Wallraven G, Kumar P, Bognar E, Manning L, Pappen B O, Adams N, Senzer N, Nemunaitis J. Phase I I study of Vigil® DNA engineered immunotherapy as maintenance in advanced stage ovarian cancer. Gynecol Oncol. 2016; 143(3):504-10.
8. Block M S, Dietz A B, Gustafson M P, Kalli K R, Erskine C L, Youssef B, Vijay G V, Allred J B, Pavelko K D, Strausbauch M A, Lin Y, Grudem M E, Jatoi A, Klampe C M, Wahner-Hendrickson A E, Weroha S J, Glaser G E, Kumar A, Langstraat C L, Solseth M L, Deeds M C, Knutson K L, Cannon M J. Thl7-inducing autologous dendritic cell vaccination promotes antigen-specific cellular and humoral immunity in ovarian cancer patients. Nat Commun. 2020; 1 1(1):5173.
9. Brown T A, Byrd K, Vreeland T J, Clifton G T, Jackson D O, Hale D F, Herbert G S, Myers J W, Greene J M, Berry J S, Martin J, Elkas J C, Conrads T P, Darcy K M, Hamilton C A, Maxwel G L, Peoples G E. Final analysis of a phase I/Ila trial of the folate-binding protein-derived E39 peptide vaccine to prevent recurrence in ovarian and endometrial cancer patients. Cancer Med. 2019; 8(10):4678-87.
10. Wick D A, Webb J R, Nielsen J S, Martin S D, Kroeger D R, Milne K, Castellarin M, Twumasi-Boateng K, Watson P H, Holt R A, Nelson B H. Surveillance of the tumor mutanome by T cells during progression from primary to recurrent ovarian cancer. Clinical cancer research: an official journal of the American Association for Cancer Research. 2014; 20(5):1125-34.
11. Schumacher T N, Schreiber R D. Neoantigens in cancer immunotherapy. Science (New York, NY). 2015; 348(6230):69-74.
12. McNeish I A. Neoantigens in Ovarian Cancer: Embarrassment of Riches or Needles in a Haystack? Clinical cancer research: an official journal of the American Association for Cancer Research. 2018; 24(22):5493-5.
13. Tanyi J L, Bobisse S, Ophir E, Tuyaerts S, Roberti A, Genolet R, Baumgartner P, Stevenson B J, Iseli C, Dangaj D, Czerniecki B, Semilietof A, Racle J, Michel A, Xenarios I, Chiang C, Monos D S, Torigian D A, Nisenbaum H L, Michielin O, June C H, Levine B L, Powell D J, Jr., Gfeller D, Mick R, Dafni U, Zoete V, Harari A, Coukos G, Kandalaft L E. Personalized cancer vaccine effectively mobilizes antitumor T cell immunity in ovarian cancer. Science translational medicine. 2018; 10(436).
14. Zhang A W, McPherson A, Milne K, Kroeger D R, Hamilton P T, Miranda A, Funnell T, Little N, de Souza C P E, Laan S, LeDoux S, Cochrane D R, Lim J L P, Yang W, Roth A, Smith M A, Ho J, Tse K, Zeng T, Shlafman I, Mayo M R, Moore R, Failmezger H, Heindl A, Wang Y K, Bashashati A, Grewal D S, Brown S D, Lai D, Wan A N C, Nielsen C B, Huebner C, Tessier-Cloutier B, Anglesio M S, Bouchard-Côté A, Yuan Y, Wasserman W W, Gilks C B, Karnezis A N, Aparicio S, McAlpine J N, Huntsman D G, Holt R A, Nelson B H, Shah S P. Interfaces of Malignant and Immunologic Clonal Dynamics in Ovarian Cancer. Cell. 2018; 173(7):1755-69.e22.
15. Sarivalasis A, Morotti M, Mulvey A, Imbimbo M, Coukos G. Cell therapies in ovarian cancer. Ther Adv Med Oncol. 2021; 13:17588359211008399.
16. Ning F, Cole C B, Annunziata C M. Driving Immune Responses in the Ovarian Tumor Microenvironment. Front Oncol. 2020; 10:604084.
17. Andtbacka R H, Kaufman H L, Collichio F, Amatruda T, Senzer N, Chesney J, Delman K A, Spitler L E, Puzanov I, Agarwala S S, Milhem M, Cranmer L, Curti B, Lewis K, Ross M, Guthrie T, Linette G P, Daniels G A, Harrington K, Middleton M R, Miller W H, Jr., Zager J S, Ye Y, Yao B, Li A, Doleman S, VanderWalde A, Gansert J, Coffin R S. Talimogene Laherparepvec Improves Durable Response Rate in Patients With Advanced Melanoma. J Clin Oncol. 2015; 33(25):2780-8.
18. Haitz K, Khosravi H, Lin J Y, Menge T, Nambudiri V E. Review of talimogene laherparepvec: A first-in-class oncolytic viral treatment of advanced melanoma. Journal of the American Academy of Dermatology. 2020; 83(1):189-96.
19. Dochez V, Caillon H, Vaucel E, Dimet J, Winer N, Ducarme G. Biomarkers and algorithms for diagnosis of ovarian cancer: CA125, HE4, RMI and ROMA, a review. J Ovarian Res. 2019; 12(1):28.
20. Gandhi T, Bhatt H. Cancer Antigen 125. 2020. In: StatPearls [Internet]. Treasure Island (FL): StatPearls Publishing, LLC. Available from: https://www.ncbi.nlm.nih.gov/books/NBK562245/.
21. Bast R C, Jr., Feeney M, Lazarus H, Nadler L M, Colvin R B, Knapp R C. Reactivity of a monoclonal antibody with human ovarian carcinoma. J Clin Invest. 1981; 68(5):1331-7.
22. Aithal A, Rauth S, Kshirsagar P, Shah A, Lakshmanan I, Junker W M, Jain M, Ponnusamy M P, Batra S K. MUC16 as a novel target for cancer therapy. Expert opinion on therapeutic targets. 2018; 22(8):675-86.
23. Liu J F, Moore K N, Birrer M J, Berlin S, Matulonis U A, Infante J R, Wolpin B, Poon K A, Firestein R, Xu J, Kahn R, Wang Y, Wood K, Darbonne W C, Lackner M R, Kelley S K, Lu X, Choi Y J, Maslyar D, Humke E W, Burris H A. Phase I study of safety and pharmacokinetics of the anti-MUC16 antibody-drug conjugate DMUC5754A in patients with platinum-resistant ovarian cancer or unresectable pancreatic cancer. Annals of oncology: official journal of the European Society for Medical Oncology. 2016; 27(11):2124-30.
24. Paijens S T, Leffers N, Daemen T, Helfrich W, Boezen H M, Cohlen B J, Melief C J, de Bruyn M, Nijman H W. Antigen-specific active immunotherapy for ovarian cancer. The Cochrane database of systematic reviews. 2018; 9(9):Cd007287.
25. Sabbatini P, Harter P, Scambia G, Sehouli J, Meier W, Wimberger P, Baumann K H, Kurzeder C, Schmalfeldt B, Cibula D, Bidzinski M, Casado A, Martoni A, Colombo N, Holloway R W, Selvaggi L, Li A, del Campo J, Cwiertka K, Pinter T, Vermorken J B, Pujade-Lauraine E, Scartoni S, Bertolotti M, Simonelli C, Capriati A, Maggi C A, Berek J S, Pfisterer J. Abagovomab as maintenance therapy in patients with epithelial ovarian cancer: a phase III trial of the AGO OVAR, COGI, GINECO, and GEICO—the MIMOSA study. J Clin Oncol. 2013; 31(12):1554-61.
26. Berek J S, Taylor P T, Gordon A, Cunningham M J, Finkler N, Orr J, Jr., Rivkin S, Schultes B C, Whiteside T L, Nicodemus C F. Randomized, placebo-controlled study of oregovomab for consolidation of clinical remission in patients with advanced ovarian cancer. J Clin Oncol. 2004; 22(17):3507-16.
27. Battaglia A, Fossati M, Buzzonetti A, Scambia G, Fattorossi A. A robust immune system conditions the response to abagovomab (anti-idiotypic monoclonal antibody mimicking the CA125 protein) vaccination in ovarian cancer patients. Immunology letters. 2017; 191:35-9.
28. Chekmasova A A, Rao T D, Nikhamin Y, Park K J, Levine D A, Spriggs D R, Brentjens R J. Successful eradication of established peritoneal ovarian tumors in SCID-Beige mice following adoptive transfer of T cells genetically targeted to the MUC16 antigen. Clinical cancer research: an official journal of the American Association for Cancer Research. 2010; 16(14):3594-606.
29. Koneru M, O'Cearbhaill R, Pendharkar S, Spriggs D R, Brentjens R J. A phase I clinical trial of adoptive T cell therapy using IL-12 secreting MUC-16(ecto) directed chimeric antigen receptors for recurrent ovarian cancer. Journal of translational medicine. 2015; 13:102.
30. Yin B W, Lloyd K O. Molecular cloning of the CA125 ovarian cancer antigen: identification as a new mucin, MUC16. The Journal of biological chemistry. 2001; 276(29):27371-5.
31. Thapi D, Binder D, Rosales N, Yan X J, Lash A E, Spriggs D R. Abstract 2033: Transcription factors that regulate MUC16 expression in ovarian cancer. Cancer Research. 2011; 71(8 Supplement):2033-.
32. Zhang M X, Hong S S, Cai Q Q, Zhang M, Chen J, Zhang X Y, Xu C J. Transcriptional control of the MUC16 promoter facilitates follicle-stimulating hormone peptide-conjugated shRNA nanoparticle-mediated inhibition of ovarian carcinoma in vivo. Drug delivery. 2018; 25(1):797-806.
33. Yue E, Yang G, Yao Y, Wang G, Mohanty A, Fan F, Zhao L, Zhang Y, Mirzapoiazova T, Walser T C, Rodriguez-Rodriguez L, Fong Y, Salgia R, Wang E W. Targeting CA-125 Transcription by Development of a Conditionally Replicative Adenovirus for Ovarian Cancer Treatment. Cancers (Basel). 2021; 13(17).
34. Neukirch L, Fougeroux C, Andersson A C, Holst P J. The potential of adenoviral vaccine vectors with altered antigen presentation capabilities. Expert Rev Vaccines. 2020; 19(1):25-41.
35. Hasanpourghadi M, Novikov M, Ertl H C J. COVID-19 Vaccines Based on Adenovirus Vectors. Trends Biochem Sci. 2021; 46(5):429-30.
36. González-Pastor R, Ashshi A M, El-Shemi A G, Dmitriev T P, Kashentseva E A, Lu Z H, Goedegebuure S P, Podhajcer O L, Curiel D T. Defining a murine ovarian cancer model for the evaluation of conditionally-replicative adenovirus (CRAd) virotherapy agents. J Ovarian Res. 2019; 12(1):18.
37. Brahmer J R, Tykodi S S, Chow L Q, Hwu W J, Topalian S L, Hwu P, Drake C G, Camacho L H, Kauh J, Odunsi K, Pitot H C, Hamid O, Bhatia S, Martins R, Eaton K, Chen S, Salay T M, Alaparthy S, Grosso J F, Korman A J, Parker S M, Agrawal S, Goldberg S M, Pardoll D M, Gupta A, Wigginton J M. Safety and activity of anti-PD-L1 antibody in patients with advanced cancer. The New England journal of medicine. 2012; 366(26):2455-65.
38. Boyiadzis M M, Kirkwood J M, Marshall J L, Pritchard C C, Azad N S, Gulley J L. Significance and implications of FDA approval of pembrolizumab for biomarker-defined disease. J Immunother Cancer. 2018; 6(1):35.
39. Lawrence M S, Stojanov P, Polak P, Kryukov G V, Cibulskis K, Sivachenko A, Carter S L, Stewart C, Mermel C H, Roberts S A, Kiezun A, Hammerman P S, McKenna A, Drier Y, Zou L, Ramos A H, Pugh T J, Stransky N, Helman E, Kim J, Sougnez C, Ambrogio L, Nickerson E, Shefler E, Cortés ML, Auclair D, Saksena G, Voet D, Noble M, DiCara D, Lin P, Lichtenstein L, Heiman D I, Fennell T, Imielinski M, Hernandez B, Hodis E, Baca S, Dulak A M, Lohr J, Landau D A, Wu C J, Melendez-Zajgla J, Hidalgo-Miranda A, Koren A, McCarroll S A, Mora J, Crompton B, Onofrio R, Parkin M, Winckler W, Ardlie K, Gabriel S B, Roberts C W M, Biegel J A, Stegmaier K, Bass A J, Garraway L A, Meyerson M, Golub T R, Gordenin D A, Sunyaev S, Lander E S, Getz G. Mutational heterogeneity in cancer and the search for new cancer-associated genes. Nature. 2013; 499(7457):214-8.
40. Vasey P A, Shulman L N, Campos S, Davis J, Gore M, Johnston S, Kim D H, O'Neill V, Siddiqui N, Seiden M V, Kaye S B. Phase I trial of intraperitoneal injection of the E1B-55-kd-gene-deleted adenovirus ONYX-015 (dl1520) given on days 1 through 5 every 3 weeks in patients with recurrent/refractory epithelial ovarian cancer. J Clin Oncol. 2002; 20(6):1562-9.
41. Bischoff J R, Kim D H, Williams A, Heise C, Horn S, Muna M, Ng L, Nye J A, Sampson-Johannes A, Fattaey A, McCormick F. An adenovirus mutant that replicates selectively in p53-deficient human tumor cells. Science (New York, NY). 1996; 274(5286):373-6.
42. Sauthoff H, Hu J, Maca C, Goldman M, Heitner S, Yee H, Pipiya T, Rom W N, Hay J G. Intratumoral spread of wild-type adenovirus is limited after local injection of human xenograft tumors: virus persists and spreads systemically at late time points. Human gene therapy. 2003; 14(5):425-33.
43. Harrison D, Sauthoff H, Heitner S, Jagirdar J, Rom W N, Hay J G. Wild-type adenovirus decreases tumor xenograft growth, but despite viral persistence complete tumor responses are rarely achieved—deletion of the viral Elb-19-kD gene increases the viral oncolytic effect. Human gene therapy. 2001; 12(10):1323-32.
44. Martin S D, Brown S D, Wick D A, Nielsen J S, Kroeger D R, Twumasi-Boateng K, Holt R A, Nelson B H. Low Mutation Burden in Ovarian Cancer May Limit the Utility of Neoantigen-Targeted Vaccines. PLoS One. 2016; 11(5):e0155189.
45. McCloskey C W, Rodriguez G M, Galpin K J C, Vanderhyden B C. Ovarian Cancer Immunotherapy: Preclinical Models and Emerging Therapeutics. Cancers (Basel). 2018; 10(8).
46. McCloskey C W, Goldberg R L, Carter L E, Gamwell L F, Al-Hujaily E M, Collins O, Macdonald E A, Garson K, Daneshmand M, Carmona E, Vanderhyden B C. A new spontaneously transformed syngeneic model of high-grade serous ovarian cancer with a tumor-initiating cell population. Front Oncol. 2014; 4:53.
47. Flint J, Shenk T. Adenovirus E1A protein paradigm viral transactivator. Annual review of genetics. 1989; 23:141-61.
48. Frisch S M, Mymryk J S. Adenovirus-5 E1A: paradox and paradigm. Nature reviews Molecular cell biology. 2002; 3(6):441-52.
49. Moran E, Mathews M B. Multiple functional domains in the adenovirus E1A gene. Cell. 1987; 48(2):177-8.
50. Steffens S, Frank S, Fischer U, Heuser C, Meyer K L, Dobberstein K U, Rainov N G, Kramm C M. Enhanced green fluorescent protein fusion proteins of herpes simplex virus type 1 thymidine kinase and cytochrome P450 4B1: applications for prodrug-activating gene therapy. Cancer gene therapy. 2000; 7(5):806-12.
51. Loimas S, Wahlfors J, Jänne J. Herpes simplex virus thymidine kinase-green fluorescent protein fusion gene: new tool for gene transfer studies and gene therapy. Biotechniques. 1998; 24(4):614-8.
52. Louis N, Evelegh C, Graham F L. Cloning and sequencing of the cellular-viral junctions from the human adenovirus type 5 transformed 293 cell line. Virology. 1997; 233(2):423-9.
53. Kaufman H L, Ruby C E, Hughes T, Slingluff C L, Jr. Current status of granulocyte-macrophage colony-stimulating factor in the immunotherapy of melanoma. J Immunother Cancer. 2014; 2:11.
54. Ribas A, Dummer R, Puzanov I, VanderWalde A, Andtbacka R H I, Michielin O, Olszanski A J, Malvehy J, Cebon J, Fernandez E, Kirkwood J M, Gajewski T F, Chen L, Gorski K S, Anderson A A, Diede S J, Lassman M E, Gansert J, Hodi F S, Long G V. Oncolytic Virotherapy Promotes Intratumoral T Cell Infiltration and Improves Anti-PD-1 Immunotherapy. Cell. 2017; 170(6):1109-19.e10.
55. Roviello G, Zanotti L, Correale P, Gobbi A, Wigfield S, Guglielmi A, Pacifico C, Generali D. Is still there a role for IL-2 for solid tumors other than melanoma or renal cancer? Immunotherapy. 2017; 9(1):25-32.
56. Rosenberg S A. IL-2: the first effective immunotherapy for human cancer. J Immunol. 2014; 192(12):5451-8.
57. Sznol M, Dutcher J P, Atkins M B, Rayner A R, Margolin K A, Gaynor E R, Weiss G R, Aronson F, Parkinson D R, Hawkins M J. Review of interleukin-2 alone and interleukin-2/LAK clinical trials in metastatic malignant melanoma. Cancer Treat Rev. 1989; 16 Suppl A:29-38.
58. Parmiani G. An explanation of the variable clinical response to interleukin 2 and LAK cells. Immunol Today. 1990; 11(4):113-5.
59. Yang J C, Rosenberg S A. Adoptive T-Cell Therapy for Cancer. Adv Immunol. 2016; 130:279-94.
60. Cheng E M, Tsarovsky N W, Sondel P M, Rakhmilevich A L. Interleukin-12 as an in situ cancer vaccine component: a review. Cancer Immunol Immunother. 2022.
61. Bashyam H. Interleukin-12: a master regulator. J Exp Med. 2007; 204(5):969.
62. Zhao J, Zhao J, Perlman S. Differential effects of IL-12 on Tregs and non-Treg T cells: roles of IFN-7, IL-2 and IL-2R. PLoS One. 2012; 7(9):e46241.
63. Watkins S K, Egilmez N K, Suttles J, Stout R D. IL-12 rapidly alters the functional profile of tumor-associated and tumor-infiltrating macrophages in vitro and in vivo. J Immunol. 2007; 178(3):1357-62.
64. Waickman A T, Ligons D L, Hwang S, Park J Y, Lazarevic V, Sato N, Hong C, Park J H. CD4 effector T cell differentiation is controlled by IL-15 that is expressed and presented in trans. Cytokine. 2017; 99:266-74.
65. Tao Q, Chen T, Tao L, Wang H, Pan Y, Xiong S, Zhai Z. IL-15 improves the cytotoxicity of cytokine-induced killer cells against leukemia cells by upregulating CD3+CD56+ cells and downregulating regulatory T cells as well as IL-35. J Immunother. 2013; 36(9):462-7.
66. Cheuk A T, Mufti G J, Guinn B A. Role of 4-1BB:4-1BB ligand in cancer immunotherapy. Cancer gene therapy. 2004; 11(3):215-26.
67. Martinez-Perez A G, Perez-Trujillo J J, Garza-Morales R, Loera-Arias M J, Saucedo-Cardenas O, Garcia-Garcia A, Rodriguez-Rocha H, Montes-de-Oca-Luna R. 4-1BBL as a Mediator of Cross-Talk between Innate, Adaptive, and Regulatory Immunity against Cancer. Int J Mol Sci. 2021; 22(12).
68. Etxeberria I, Glez-Vaz J, Teijeira Á, Melero I. New emerging targets in cancer immunotherapy: CD137/4-1BB costimulatory axis. ESMO Open. 2020; 4(Suppl 3):e000733.
69. Fisher K. Striking out at disseminated metastases: the systemic delivery of oncolytic viruses. Curr Opin Mol Ther. 2006; 8(4):301-13.
70. Gonzalez-Pastor R, Goedegebuure P S, Curiel D T. Understanding and addressing barriers to successful adenovirus-based virotherapy for ovarian cancer. Cancer gene therapy. 2021; 28(5):375-89.
71. Chaurasiya S, Fong Y, Warner S G. Oncolytic Virotherapy for Cancer: Clinical Experience. Biomedicines. 2021; 9(4).
72. Roby K F, Taylor C C, Sweetwood J P, Cheng Y, Pace J L, Tawfik O, Persons D L, Smith P G, Terranova P F. Development of a syngeneic mouse model for events related to ovarian cancer. Carcinogenesis. 2000; 21(4):585-91.
73. McCloskey C, Goldberg R, Carter L, Gamwell L, Al-Hujaily E, Collins O, Macdonald E, Garson K, Daneshmand M, Carmona E, Vanderhyden B. A New Spontaneously Transformed Syngeneic Model of High-Grade Serous Ovarian Cancer with a Tumor-Initiating Cell Population. Frontiers in Oncology. 2014; 4(53).
74. Liu Z, Ravindranathan R, Kalinski P, Guo Z S, Bartlett D L. Rational combination of oncolytic vaccinia virus and PD-L1 blockade works synergistically to enhance therapeutic efficacy. Nat Commun. 2017; 8:14754.
75. Moynihan K D, Opel C F, Szeto G L, Tzeng A, Zhu E F, Engreitz J M, Williams R T, Rakhra K, Zhang M H, Rothschilds A M, Kumari S, Kelly R L, Kwan B H, Abraham W, Hu K, Mehta N K, Kauke M J, Suh H, Cochran J R, Lauffenburger D A, Wittrup K D, Irvine D J. Eradication of large established tumors in mice by combination immunotherapy that engages innate and adaptive immune responses. Nat Med. 2016; 22(12):1402-10.
76. Wang G, Kang X, Chen K S, Jehng T, Jones L, Chen J, Huang X F, Chen S Y. An engineered oncolytic virus expressing PD-L1 inhibitors activates tumor neoantigen-specific T cell responses. Nat Commun. 2020; 11(1):1395.
77. Agallou M, Karagouni E. Detection of Antigen-specific T cells in Spleens of Vaccinated Mice Applying (3)[H]-Thymidine Incorporation Assay and Luminex Multiple Cytokine Analysis Technology. Bio Protoc. 2019; 9(11):e3252.
78. Kanerva A, Wang M, Bauerschmitz G J, Lam J T, Desmond R A, Bhoola S M, Barnes M N, Alvarez R D, Siegal G P, Curiel D T, Hemminki A. Gene transfer to ovarian cancer versus normal tissues with fiber-modified adenoviruses. Mol Ther. 2002; 5(6):695-704.
79. Atasheva S, Yao J, Shayakhmetov D M. Innate immunity to adenovirus: lessons from mice. FEBS Lett. 2019; 593(24):3461-83.
80. Shayakhmetov D M, Gaggar A, Ni S, L1 ZY, Lieber A. Adenovirus binding to blood factors results in liver cell infection and hepatotoxicity. J Virol. 2005; 79(12):7478-91.
81. Chesney J, Puzanov I, Collichio F, Singh P, Milhem M M, Glaspy J, Hamid O, Ross M, Friedlander P, Garbe C, Logan T F, Hauschild A, Lebbé C, Chen L, Kim J J, Gansert J, Andtbacka R H I, Kaufman H L. Randomized, Open-Label Phase I I Study Evaluating the Efficacy and Safety of Talimogene Laherparepvec in Combination With Ipilimumab Versus Ipilimumab Alone in Patients With Advanced, Unresectable Melanoma. J Clin Oncol. 2018; 36(17):1658-67.
82. Liang M. Oncorine, the World First Oncolytic Virus Medicine and its Update in China. Current cancer drug targets. 2018; 18(2):171-6.
83. Miller D L, Myers C L, Rickards B, Coller H A, Flint S J. Adenovirus type 5 exerts genome-wide control over cellular programs governing proliferation, quiescence, and survival. Genome Biol. 2007; 8(4):R58.
84. Hidaka C, Milano E, Leopold P L, Bergelson J M, Hackett N R, Finberg R W, Wickham T J, Kovesdi I, Roelvink P, Crystal R G. CAR-dependent and CAR-independent pathways of adenovirus vector-mediated gene transfer and expression in human fibroblasts. J Clin Invest. 1999; 103(4):579-87.
85. Godoy H E, Khan A N, Vethanayagam R R, Grimm M J, Singel K L, Kolomeyevskaya N, Sexton K J, Parameswaran A, Abrams S I, Odunsi K, Segal B H. Myeloid-derived suppressor cells modulate immune responses independently of NADPH oxidase in the ovarian tumor microenvironment in mice. PLoS One. 2013; 8(7):e69631.
86. Baert T, Vankerckhoven A, Riva M, Van Hoylandt A, Thirion G, Holger G, Mathivet T, Vergote I, Coosemans A. Myeloid Derived Suppressor Cells: Key Drivers of Immunosuppression in Ovarian Cancer. Front Immunol. 2019; 10:1273.
87. Komura N, Mabuchi S, Shimura K, Yokoi E, Kozasa K, Kuroda H, Takahashi R, Sasano T, Kawano M, Matsumoto Y, Kodama M, Hashimoto K, Sawada K, Kimura T. The role of myeloid-derived suppressor cells in increasing cancer stem-like cells and promoting PD-L1 expression in epithelial ovarian cancer. Cancer Immunol Immunother. 2020; 69(12):2477-99.
88. Scurr M, Pembroke T, Bloom A, Roberts D, Thomson A, Smart K, Bridgeman H, Adams R, Brewster A, Jones R, Gwynne S, Blount D, Harrop R, Hills R, Gallimore A, Godkin A. Low-Dose Cyclophosphamide Induces Antitumor T-Cell Responses, which Associate with Survival in Metastatic Colorectal Cancer. Clinical cancer research: an official journal of the American Association for Cancer Research. 2017; 23(22):6771-80.
89. Sobhani N, Tardiel-Cyril D R, Davtyan A, Generali D, Roudi R, L1 Y. CTLA-4 in Regulatory T Cells for Cancer Immunotherapy. Cancers (Basel). 2021; 13(6).
90. Wei S C, Levine J H, Cogdill A P, Zhao Y, Anang N A S, Andrews M C, Sharma P, Wang J, Wargo J A, Pe'er D, Allison J P. Distinct Cellular Mechanisms Underlie Anti-CTLA-4 and Anti-PD-1 Checkpoint Blockade. Cell. 2017; 170(6):1120-33.e17.
91. Locke F L, Pidala J, Storer B, Martin P J, Pulsipher M A, Chauncey T R, Jacobsen N, Kröger N, Walker I, Light S, Shaw B E, Beato F, Laport G G, Nademanee A, Keating A, Socie G, Anasetti C. CD25 Blockade Delays Regulatory T Cell Reconstitution and Does Not Prevent Graft-versus-Host Disease After Allogeneic Hematopoietic Cell Transplantation. Biol Blood Marrow Transplant. 2017; 23(3):405-11.
92. Tanaka A, Sakaguchi S. Targeting Treg cells in cancer immunotherapy. Eur J Immunol. 2019; 49(8):1140-6.

INFORMAL SEQUENCE LISTING

SEQ ID NO: 1 (1040 base pair promoter)
taggtaggggctctatctgcatctttctttcttttttttttctttcccttcctcccttcctcactccctcggtcctctctttctttccttttctt
tcttccttcctcccttcctccctccctccctctctctttctctcttttttctttccttctttctttctttctctcttccttcccccctccctcct
tccttcctttctctttctttctctttctttctttttttccttccttccttccttctttctctttctctccctcccttccttccttccttccttcc
ttccttcctttctttctttctttctttctttctttctttctttctttctttctttctttcttccttccttccttccttccttccttccttccttc
cttcctttcttttctttctttctctttctttttgagacagagctcttattacccatgctggagtgcagtggtgtgaccttggcttactgcaacat
ctgcctcctagggtcaagtgattctcctgcctcagcctcctaagtagctgggattacagacacatgccaccacacccaatatttatttttattaa
aattttttttaaaattatttttaaaaaattaaaaataattttgtatttttagtagagacggggtttctccatgttggtcaggctgatctcaaact
cccaacctcaggtgatcctcccacctcacctcccaaagtgctgggattacaggcatgagccaccgtgcccagcctggttcctggtttctaagaca
tcacacacacacacacacacacacacacacactcacacactcagagagagagagagagagaggatcattaagacatgatacactaa
gaaattctattctgcagacactgagaatccgttaaaaagtttgaagggaagaattgagatcatcaggtgtttatttgaggaaattgtct
gtggttgaactatcctttcctttctctccctgagatttggtcttctcaattagaagcgttgcacaattcccccaacctccatacatacgg
cagctcttctag
SEQ ID NO: 2 (646 base pair promoter)
taggtaggggctctatctgcatctttctttctttttttctttctttcccttcctcccttcctcactccctcggtcctctctttctttccttttct
ttcttccttcctcccttcctccctccctccctctctctttctctctttctttctttccttctttctttctttctctcttccttccctccctccct
ccttccttcctttctctttctttctctttctttctttttttccttccttccttccttctttctctttctctccctcccttccttccttccttcct
tccttccttcctttctttctttctttctttctttctttctttctttctttctttctttctttcttccttccttccttccttccttccttccttcc
ttccttcctttcttttctttctttctctttctttttgagacagagctcttattacccatgctggagtgcagtggtgtgaccttggcttactgcaa
catctgcctcctagggtcaagtgattctcctgcctcagcctcctaagtagctgggattacagacacatgccaccacacccaatatttatttttat
taaaattttttttaaaattatttttaaaaaattaaaaataattttgtatttttagtagagacggggtttctccatg
SEQ ID NO: 3 (5,033 base pair fragment of the MUC16 gene)
ttccatccttttctcctcacaggaactcacgagcctggggagaattcgtcatccccacatggaacctcagtatggccaacaggcag
ccctctggtagaaaaatcaacaatcagtcctgcaggtatgctccaatctcatctcctccacattgttatggtctcccggagctttgtta
atctctttgaagacgaacatttttgatcttttccataggggaagtgcaagggacgcctgtgttggagtttgctccccagctacagggc
tccctaactttttatttaacctccagctttatgaccatgtacaagtatgcaattctaatctttatcttgttttgagattctaatccagagtagt
tgggggacaggaagcccagcctataacccctgatttttttttttttttttgagacagagtctcattctgtcacccaggctggagtgcag
tggcacaatctcagctgactgcaacccctgcctcccgagtttaagcaattttggtgtctcagcctcctgagtagctgggattacagg
cgcctgccaccacccccggctaatttttgtattttttggtagagacggagtttcaccatgttggtcaggctggtcttgaactcctgacc
tcaagtaatctgctctcctcaggactctcttggaaggatttttttttttttttttttttttagatggaattctgctctttgtctcccaggctgga
gtgcaatggtgcgatcttggctcactgtaacctccgcctcccaggttcaagtgattctcctgtctcagcctccggagtagctggaat
tacagccacccgccaccacgcccagctagtttttgtatttttagtagagacggggtttaaccatgttggccaggctggtcttgaactc
ctgacctcaggtgatccgcccgctttggcctcccaaagtgctgggattacaggcgtgagccactgcgcccagcaggaactctta
gttcagaagaagagtgagctaataatgtatatgtcttatagcacttttttgcatctaagttgttttatttgggatcagaagacttttattttct
tttttaaaatttagcttttattttaagtttaggggtacatgtgcaggcttgttatataggtaaacttttgccattggggttggttttacagatt
atttcatcacccaggtattaagcctagtatccattagttatttttcctgatcttctccctcctcccaccctccaccctccaataggccctt
aattaaactaaagagcttccgcacagtaaaggaaactatcaacagagtaaacagacaatctacagaatgggagaaaagctttaca
aactttgcatctgacaaaggtctaatatccagcatctaaaagaaacgtgccgggtgtggtggctcacacctgtaatcccagcacttt
ggcggatcacctgaggttgggagttcgagaccagcctgcccaacatggagaaagccaatctctactaaaaatacaaaaattagc
cgggcatggtggcacatgcctgtaattccagctactccaggggctgaggcagaagaatcgcttgaacccggaaggtggaggtt
gcagtgagccgagattgcaccactgcactccagcctgggtgacagagtgagactccatctcaaaaaaaataaaaaataaaaaat
aaacgaatttataatttaaaaaaccagcaaaaaacaaaacaaacaaacaaaagaaacattacacagtgggcaaaggacgtggtt
cttcaatcattttgacctggataaaattggtagctcctgagtagaaaagaagattgggaaagcatgaggaatgaattatagtttcact
taccccaccggcctagggaaaaactcaattcctaggagacgcagcttagaaccaaggtgggcctcatcttgagagaaatccatg
agccatgcctatcttagtctgtattgcattgcttatagcagaacagctgaaactgggtaatttacaaagaaaaataatgtatctattac
agatatggaggctggaaagttgaaatccagaggggcacacctggtgagagccctgttgctggtggggactctgtggcatccag
aggtagacagggcatcacacgtcaagacagctgagtgcgctaatgtgatagcacaggtctcacttcctattttaaagcttttcttttct
ttttttgtttgagatggagtctcaatctgtcacccaggatggagtgcagtgctgcgatcttggctcactgcaacctccacctcctggg
ttcaagtgattttcctctcttagcctcctgagtagctgggaccacaggtgcgcaccactatgtctggctaatttttgtatttttttgtaga
gacatggtttcaccatgttggccaggctggtctcgaattcctgacttcaagtaatccacccacctcagcctcccaaagtgctgggat
tacaagcatgagccaccatgcatggcctaaagcttcttttaaagccaccaagtcccttcccatgttagcccactaatccatgggtta
gtcatgaatggattaatctattcatacggacagagccctcatcacccaatcacctcttaaaggccccacctctcaatactgccacac
tggggattaagtttcaacagagttttggaggggacattcaaatcatagtaatgcccaaagtgaaaaatcttccctgcacttttccctc
aacaaaaacagccagagatagtgagctgccaggaaattcttttttttttcctcttctgtcctaaatcagcatcgctagacctttacatga
ttcaacctcatcttcttcaccctctgggtcatgaaattttatttatttatttattattttcttgggacagactctggctctgtcgcccaggctg
aagtgcagtggtgtgatcttggctcactgcaacctccgcctcccgggttcaagcgattctcctgcctcagcctcctgagtagctgg
gattacaggtgggcgccaccacacccagctaattttttgtatttttagtagagatggggtttcaccatattagccaggatggtctccat
ctcttgacctcgtgatctgcccacctcagcctcccaaaatgctgggattacaggcatgagacaccacgcccagcaggccagggt
catgagattttaatcaagagcaacttccactgattcctgagagtgcatctgtgggcccctgctctgatctgaacagaagtgccgtgt
cttctctgacctccacttctcaattcaagagccttagtatctgccagtatcacacactgagcattagctccatctcatgggggtgtag
gtaggggctctatctgcatctttctttctttttttctttctttcccttcctcccttcctcactccctcggtcctctctttctttccttttcttt
cttccttcctcccttcctccctcccccctctctctttctctctttctttctttccttctttctttctttctctcttccttccctccctccctcct
tccttcctttctctttctttctctttctttctttttttccttccttccttccttctttctctttctctccctcccttccttccttccttccttcc
ttccttcctttctttctttctttctttctttctttctttcttttttctttctttctttcttccttccttccttccttccttccttccttccttcc
ttcctttcttttctttctttctctttctttttgagacagagctcttattacccatgctggagtgcagtggtgtgaccttggcttactgcaacat
ctgcctcctagggtcaagtgattctcctgcctcagcctcctaagtagctgggattacagacacatgccaccacacccaatatttatttttatta
aaattttttttaaaattatttttaaaaaattaaaaataattttgtatttttagtagagacggggtttctccatgttggtcaggctgatctcaaac
tcccaacctcaggtgatcctcccacctcacctcccaaagtgctgggattacaggcatgagccaccgtgcccagcctggttcctggtttctaagac
atcacacacacacacacacacacacacacacactcacacactcagagagagagagagagagaggatcattaagacatgatacactaaga
aattctattctgcagacactgagaatccgttaaaaagtttgaagggaagaattgagatcatcaggtgtttatttgaggaaattgtctgt
ggttgaactatcctttcctttctctccctgagatttggtcttctcaattagaagcgttgcacaattcccccaacctccatacatacggca
gctcttctagacacaggttttcccaggtcaaatgcggggaccccagccatatctcccaccctgagaaattttggagtttcagggag
ctcagaagctctgcagaggccaccctctctgaggggattcttcttagacctccatccagaggcaaatgttgacctgtccATGC
TGAAACCCTCAGGCCTTCCTGGGTCATCTTCTCCCACCCGCTCCTTGATGACA
GGGAGCAGGAGCACTAAAGCCACACCAGAAATGGATTCAGGACTGACAGGA
GCCACCTTGTCACCTAAGACATCTACAGGTGCAATCGTGGTGACAGAACATA
CTCTGCCCTTTACTTCCCCAGATAAGACCTTGGCCAGTCCTACATCTTCGGTTG
TGGGAAGAACCACCCAGTCTTTGGGGGTGATGTCCTCTGCTCTCCCTGAGTCA
ACCTCTAGAGGAATGACACACTCCGAGCAAAGAACCAGCCCATCGCTGAGTC
CCCAGGTCAATGGAACTCCCTCTAGGAACTACCCTGCTACAAGCATGGTTTCA
GGATTGAGTTCCCCAAGGACCAGGACCAGTTCCACAGAAGGAAATTTTACCA
AAGAAGCATCTACATACACACTCACTGTAGAGACCACAAGTGGCCCAGTCAC
TGAGAAGTACACAGTCCCCACTGA
SEQ ID NO: 4
ACAAACTAGCAAAATAGGCTGT
SEQ ID NO: 5
CTTCTTAATGTTTTTGGCATCT
SEQ ID NO: 6
AAGAAGGTGGTGAAGCAGGC
SEQ ID NO: 7
TCCACCACCCTGTTGCTGTA
SEQ ID NO: 8 (adenovirus E1A DNA)
ATGAGACATA TTATCTGCCA CGGAGGTGTT ATTACCGAAG AAATGGCCGC
CAGTCTTTTG GACCAGCTGA TCGAAGAGGT ACTGGCTGAT AATCTTCCAC
CTCCTAGCCA TTTTGAACCA CCTACCCTTC ACGAACTGTA TGATTTAGAC
GTGACGGCCC CCGAAGATCC CAACGAGGAG GCGGTTTCGC AGATTTTTCC
CGACTCTGTA ATGTTGGCGG TGCAGGAAGG GATTGACTTA CTCACTTTTC
CGCCGGCGCC CGGTTCTCCG GAGCCGCCTC ACCTTTCCCG GCAGCCCGAG
CAGCCGGAGC AGAGAGCCTT GGGTCCGGTT TCTATGCCAA ACCTTGTACC
GGAGGTGATC GATCTTACCT GCCACGAGGC TGGCTTTCCA CCCAGTGACG
ACGAGGATGA AGAGGGTGAG GAGTTTGTGT TAGATTATGT GGAGCACCCC
GGGCACGGTT GCAGGTCTTG TCATTATCAC CGGAGGAATA CGGGGGACCC
AGATATTATG TGTTCGCTTT GCTATATGAG GACCTGTGGC ATGTTTGTCT
ACAGTCCTGT GTCTGAACCT GAGCCTGAGC CCGAGCCAGA ACCGGAGCCT
GCAAGACCTA CCCGCCGTCC TAAAATGGCG CCTGCTATCC TGAGACGCCC
GACATCACCT GTGTCTAGAG AATGCAATAG TAGTACGGAT AGCTGTGACT
CCGGTCCTTC TAACACACCT CCTGAGATAC ACCCGGTGGT CCCGCTGTGC
CCCATTAAAC CAGTTGCCGT GAGAGTTGGT GGGCGTCGCC AGGCTGTGGA
ATGTATCGAG GACTTGCTTA ACGAGCCTGG GCAACCTTTG GACTTGAGCT
GTAAACGCCC CAGGCCATAA
SEQ ID NO: 9 (adenovirus E1B DNA)
atggagcgaa gaaacccatc tgagcggggg gtacctgctg gattttctgg ccatgcatct
gtggagagcg gttgtgagac acaagaatcg cctgctactg ttgtcttccg tccgcccggc
gataataccg acggaggagc agcagcagca gcaggaggaa gccaggcggc ggcggcagga
gcagagccca tggaacccga gagccggcct ggaccctcgg gaatgaatgt tgtacagccc
gagagcatgt ccaaggtgaa cctgaacggg gtgtttgaca tgaccatgaa gatctggaag
gtgctgaggt acgatgagac ccgcaccagg tgcagaccct gcgagtgtgg cggtaaacat
attaggaacc agcctgtgat gctggatgtg accgaggagc tgaggcccga tcacttggtg
ctggcctgca cccgcgctga gtttggctct agcgatgaag atacagattg a
SEQ ID NO: 10 (adenovirus E2A DNA)
ATGGCCAGCCGCGAAGGAAATCAGTTAAGCGACCGCCACAGGGAGCACACCCCCGAACGC
GGAAGGGGTTCTGCCAGTCATCCGCCTTCCAGGTCGGACCGCAGCCCTTCGCAATCGCCT
CCACCTCTGCCCCCGAAGAGAAACACCTGTCGCCGTGTGGGGTCAGGTTCTTCTACCGAC
AGTCAGTTGGTAATGGTGAGCGAAACTTCACAATCCTCCCTGTCTCCGGAACGGAGCGAC
AGTCCTCCTCCCCCGATACCCCCCAAAAAGAAGCCAAGGAAGACCAAACATGTTCCCATG
CAAGACATCAGTCAAGACAGCGAAGAAGAAAGAGAAGAGGCGCAGCTTGTAGCAGTAGGT
TTCAGCTACCCGCCCGTGCGCATCGTTGAGAAGGATGGCAAAAGAAGCATTGAAAAGATT
GCCAAAGATGATCCTCTGGCTAAAGGAGCTGCTGCCTGTACTGTTAAGAATCCGATTAGC
TTGCCCCTGGTGTCTGCTTGGGAGAAGGGCATGGAAGTGATGTGTGTACTTATGGAGAAG
TACAGGCTGGATAACGAACTTAGAACCTCTTTTAAACTAATGCCTGAGCAGCACGAACAA
TACAAGCGCATCTGTCACCAGTACGTAAACGAGGAGCACCGAGGCGTCCAGTTAACTTTC
ACTAGTCACAAGACACTGAGCACGATGATGGGAAGATTTCTGCAGGGAATGATACATTCT
TTTTCTCAGATCGCCCACCATAATTGGGAATGTACAGGCTGTGCGTTGTGGCCTCATGGC
TGCAATGATTATGAGGGCAAGCTGAAGTGCCTGCATGGCACTATTATGATTCAGAAGGAG
CAGATTATCGAGATGGATGTGGCCAGCGAGAATGGACAACGAGCCTTGAAGGAGAATCCC
GAGCGAACCAAAATCACCCAAAACCGCTGGGGCAGAAGTGTGGTGCAAATAGCCAACAAT
GACGCCCGCTGCTGTGTGAATGATGCCGGCTGCGCAGCTAACCAGTTTTCGAGCAGGTCC
TGCGGGATGTTCTATACCGAGGGTAGTAAAGCCCAGCAGGCTTTCAAGCAATATGATGCT
TTCATGAGAGCCGTGTATCCAGGAATCAGACAGGATCAGGCCAAGATGATACTAATCCCC
CTGCACTGCGATTGCAACCACAAGCCTAATTGGGTGCCCGCCATGGGTAGGCAGACCTGC
AAGATGACCCCGTTCAGCATTGCCAATGCTGAAGATCTGGATGTGGGCATGATTGCAGAT
CCCACCGTGCTGGCAAGCGTGAGACATCCGTCGCTGATGGTGTTTCAGTGCTGCAATCCG
GTGTACCGCAACTCTCGCGCTCAGAGCACTGGTCCTAATTGTGATTTCAAGATTTCGGCT
CCTGACCTGCTGGGAGCTTTGCAGCTGACCAGAAAGCTGTGGAGTGACATCCTACCCGAT
ATACCGGTTCCCAAGTTGGTGATTCCCGAATTCAAGTGGCAACCCAAGTATCAGTTCCGC
AACGTATCACTGCCCGCGGGTCATTCTGATTCTCGTCAGAACCCATTCGACTTGTAA
SEQ ID NO: 11 (adenovirus E2B DNA)
ATGGCCCTGAGCGTCAACGATTGCGCGCGCCTGACCGGCCAGAGCGTCCCGACCATGGAG
CACTTTTTGCCGCTGCGCAACATCTGGAACCGCGTCCGCGACTTTCCGCGCGCCTCCACC
ACCGCCGCCGGCATCACCTGGATGTCCAGGTACATCTACGGATATCATCGCCTTATGTTG
GAAGATCTCGCCCCCGGAGCCCCGGCCACCCTACGCTGGCCCCTCTACCGCCAGCCGCCG
CCGCACTTTTTGGTGGGATACCAGTACCTGGTGCGGACTTGCAACGACTACGTATTTGAC
TCGAGGGCTTACTCGCGTCTCAGGTACACCGAGCTCTCGCAGCCGGGTCACCAGACCGTT
AACTGGTCCGTTATGGCCAACTGCACTTACACCATCAACACGGGCGCATACCACCGCTTT
GTGGACATGGATGACTTCCAGTCTACCCTCACGCAGGTGCAGCAGGCCATATTAGCCGAG
CGCGTTGTCGCCGACCTAGCCCTGCTTCAGCCGATGAGGGGCTTCGGGGTCACACGCATG
GGAGGAAGAGGGCGCCACCTACGGCCAAACTCCGCCGCCGCCGCAGCGATAGATGCAAGA
GATGCAGGACAAGAGGAAGGAGAAGAAGAAGTGCCGGTAGAAAGGCTCATGCAAGACTAC
TACAAAGACCTGCGCCGATGTCAAAACGAAGCCTGGGGCATGGCCGACCGCCTGCGCATT
CAGCAGGCCGGACCCAAGGACATGGTGCTTCTGTCGACCATCCGCCGTCTCAAGACCGCC
TACTTTAATTACATCATCAGCAGCACCTCCGCCAGAAACAACCCCGACCGCCGCCCGCTG
CCGCCCGCCACGGTGCTCAGCCTACCTTGCGACTGTGACTGGTTAGACGCCTTTCTCGAG
AGGTTTTCCGATCCGGTCGATGCGGACTCGCTCAGGTCCCTCGGCGGCGGAGTACCTACA
CAACAATTGTTGAGATGCATCGTTAGCGCCGTATCCCTGCCGCATGGCAGCCCCCCGCCA
ACCCATAACCGGGACATGACGGGCGGCGTCTTCCAACTGCGCCCCCGCGAGAACGGCCGC
GCCGTCACCGAGACCATGCGCCGTCGCCGCGGGGAGATGATCGAGCGCTTTGTCGACCGC
CTCCCGGTGCGCCGTCGTCGCCGCCGTGTCCCCCCTCCCCCACCGCCGCCAGAAGAAGAA
GAAGGGGAGGCCCTTATGGAAGAGGAGATTGAAGAAGAAGAAGAGGCCCCTGTAGCCTTT
GAGCGCGAGGTGCGCGACACTGTCGCCGAGCTCATCCGTCTTCTGGAGGAGGAGTTAACC
GTGTCGGCGCGCAACTCCCAGTTTTTCAACTTCGCCGTGGACTTCTACGAGGCCATGGAG
CGCCTTGAGGCCTTGGGGGATATCAACGAATCCACGTTGCGACGCTGGGTTATGTACTTC
TTCGTGGCAGAACACACCGCCACCACCCTCAACTACCTCTTTCAGCGCCTGCGAAACTAC
GCCGTCTTCGCCCGGCACGTGGAGCTCAATCTCGCGCAGGTGGTCATGCGCGCCCGCGAT
GCCGAAGGGGGCGTGGTCTACAGCCGCGTCTGGAACGAGGGAGGCCTCAACGCCTTCTCG
CAGCTCATGGCCCGCATTTCCAACGACCTCGCCGCCACCGTGGAGCGAGCCGGACGCGGA
GATCTCCAGGAGGAAGAGATCGAGCAGTTCATGGCCGAGATCGCCTATCAAGACAACTCA
GGAGACGTGCAGGAGATTTTGCGCCAGGCCGCCGTCAACGACACCGAAATTGATTCTGTC
GAACTCTCTTTCAGGTTCAAGCTCACCGGGCCCGTCGTCTTCACGCAGAGGCGCCAGATT
CAGGAGATCAACCGCCGCGTCGTCGCGTTCGCCAGCAACCTACGCGCGCAGCACCAGCTC
CTGCCCGCGCGCGGCGCCGACGTGCCCCTGCCCCCTCTCCCGGCGGGTCCGGAGCCCCCC
CTACCTCCGGGGGCTCGCCCGCGTCACCGCTTTTAG
SEQ ID NO: 12 (adenovirus E3 DNA)
atgattaggt acataatcct aggtttactc acccttgcgt cagcccacgg
aaggtggatt ttaaggagcc agcctgtaat gttacattcg cagctgaagc taatgagtgc
accactctta taaaatgcac cacagaacat gaaaagctgc ttattcgcca caaaaacaaa
attggcaagt atgctgttta tgctatttgg cagccaggtg acactacaga gtataatgtt
acagttttcc agggtaaaag tcataaaact tttatgtata cttttccatt ttatgaaatg
tgcgacatta ccatgtacat gagcaaacag tataagttgt ggcccccaca aaattgtgtg
gaaaacactg gcactttctg ctgcactgct atgctaatta cagtgctcgc tttggtctgt
accctactct atattaaata caaaagcaga cgcagcttta ttgaggaaaa gaaaatgcct

What is claimed is:

1. A virus comprising a nucleic acid encoding a MUC16 promoter operably linked to an essential viral gene.

2. The virus of claim 1, wherein said MUC16 promoter is from about 20 nucleic acid residues to about 6000 nucleic acid residues in length.

3. The virus of claim 1, wherein said MUC16 promoter is from about 800 nucleic acid residues to about 1200 nucleic acid residues in length.

4. The virus of claim 1, wherein said MUC16 promoter is from about 400 nucleic acid residues to about 800 nucleic acid residues in length.

5. The virus of claim 1, wherein said MUC16 promoter comprises a nucleic acid sequence within a region from about −6000 nucleic acid residues upstream to about +200 nucleic residues downstream of a MUC16 transcription start site (TSS).

6. The virus of claim 5, wherein said nucleic acid sequence is from about −1200 nucleic acid residues upstream to about +100 nucleic acid residues downstream of the MUC16 TSS.

7. The virus of claim 5, wherein said nucleic acid sequence is from about −1200 nucleic acid residues upstream to about −500 nucleic residues upstream of the MUC16 TSS.

8. The virus of claim 1, wherein said MUC16 promoter comprises the nucleic acid sequence of SEQ ID NO:1.

9. The virus of claim 1, wherein said MUC16 promoter comprises the nucleic acid sequence of SEQ ID NO:2.

10. The virus of claim 1, wherein said virus further includes a nucleic acid sequence encoding one or more anti-cancer proteins.

11. The virus of claim 10, wherein said anti-cancer protein is a cytokine, a chemokine, an immune costimulatory molecule, an immune checkpoint inhibitor, a cytotoxic protein, a tumor suppressor, an apoptosis-inducing protein, or an anti-angiogenesis factor.

12. The virus of claim 10, wherein said anti-cancer protein is an immune-activating protein.

13. The virus of claim 12, wherein said immune-activating protein is granulocyte-macrophage colony-stimulating factor (GM-CSF), IL-2, TL-12, TL-15, B7-1, CD137L, granulocyte colony-stimulating factor receptor (G-CSF), macrophage colony-stimulating factor (M-CSF), or an anti-CD25 antibody.

14. The virus of claim 12, wherein said immune-activating protein is GM-CSF.

15. The virus of claim 10, wherein said anti-cancer protein is an apoptosis-inducing factor.

16. The virus of claim 15, wherein said apoptosis-inducting factor is TNF-related apoptosis-inducing ligand (TRATL).

17. The virus of claim 10, where in the anti-cancer protein is an immune checkpoint inhibitor.

18. The virus of claim 17, wherein the immune checkpoint inhibitor is an anti-PD-1 antibody, an anti-PD-L1 antibody, or an anti-CTLA-4 antibody.

19. The virus of claim 1, wherein said MUC16 promoter comprises one or more transactivation elements.

20. The virus of claim 1, further comprising a nucleic acid encoding a detectable protein.

21. The virus of claim 20, wherein said detectable protein is a fluorescent protein.

22. The virus of claim 1, further comprising a nucleic acid encoding thymidine kinase.

23. The virus of claim 1, wherein said virus is an oncolytic virus.

24. The virus of claim 1, wherein said essential viral gene is E1A.

25. The virus of claim 1, wherein said virus is an adenovirus, a herpes simplex virus, a vaccinia virus, a retrovirus, a measles virus, a reovirus, a coxsackievirus, a poliovirus, a Newcastle disease virus, a vesicular stomatitis virus, a Zika virus, an influenza virus, a rhinovirus, or a parvovirus.

26. The virus of claim 1, wherein said virus is an adenovirus.

27. The virus of claim 1, wherein said virus is formed by a method comprising:
i) contacting a cell with a nucleic acid encoding essential viral genes and the MUC16 promoter; and
ii) allowing said cell to express said essential viral genes.

28. The virus of claim 27, wherein said cell is a CA-125 expressing cancer cell.

29. An isolated nucleic acid encoding the virus of claim 1.

30. A pharmaceutical composition comprising a therapeutically effective amount of the virus of claim 1.

31. The pharmaceutical composition of claim 30, comprising a first virus and a second virus.

32. The pharmaceutical composition of claim 31, wherein the first virus comprises a first nucleic acid sequence encoding one or more anti-cancer protein and the second virus comprises a second nucleic acid sequence encoding one or more anti-cancer proteins.

33. The pharmaceutical composition of claim 31, further comprising a third virus.

34. The pharmaceutical composition of claim 33, wherein the third virus comprises a third nucleic acid sequence encoding one or more anti-cancer proteins.

35. A cell comprising the virus of claim 1.

36. A method of treating or preventing cancer in a subject in need thereof, said method comprising administering to said subject a therapeutically or prophylactically effective amount of the virus of claim 1.

37. The method of claim 36, wherein said cancer is a gynecological cancer.

38. The method of claim 36, wherein said cancer is ovarian cancer, uterine cancer, or cervical cancer.

39. The method of claim 36, wherein said cancer is ovarian cancer.

40. The method of claim 36, wherein said cancer expresses CA-125.

41. A method of stimulating an immune response in a subject in need thereof, said method comprising administering to said subject an effective amount of the virus of claim 1.

42. The method of claim 41, wherein the subject has or previously had a CA-125 expressing cancer.

43. The method of claim 36, wherein said virus is administered at a dose from about 10¹⁰ to about 10¹² plaque forming units (Pfu).

44. The method of claim 36, wherein said virus is administered intraperitoneally, intratumorally, intravenously, intrathecally, or intrapleurally.

45. The method of claim 44, wherein said virus is administered intraperitoneally.

46. A method of treating or preventing cancer in a subject in need thereof, said method comprising administering to said subject a therapeutically or prophylactically effective amount of a cell comprising the virus of claim 1.

47. The method of claim 46, wherein said cancer is a gynecological cancer.

48. The method of claim 46, wherein said cancer is a CA-125 expressing cancer.

49. The method of claim 46, wherein said cell is a CA-125 expressing cancer cell.

50. The method of claim 46, wherein said cell is administered intraperitoneally, intratumorally, intravenously, intrathecally, or intrapleurally.

51. The method of claim 50, wherein said cell is administered intraperitoneally.

52. A method of stimulating an immune response in a subject in need thereof, said method comprising administering to said subject an effective amount of a cell comprising the virus of claim 1.

53. The method of claim 52, wherein said subject has or previously had a CA-125 expressing cancer.

54. The method of claim 52, wherein said cell is a CA-125 expressing cancer cell.

55. A method of inhibiting proliferation of a CA-125 expressing cell, said method comprising contacting said CA-125 expressing cell with the virus of claim 1.

56. The method of claim 55, wherein said CA-125 expressing cell is in a subject having cancer.

57. The method of claim 56, wherein said cancer is a gynecological cancer.

58. The method of claim 56, wherein said cancer is ovarian cancer, uterine cancer, or cervical cancer.

59. The method of claim 55, wherein said CA-125 expressing cell is a CA-125 expressing cancer cell.
